(19) United States
(12) Patent Application Publication
(10) Pub. No.: US 2004/0235855 A1
(43) Pub. Date: Nov. 25, 2004

(54) Phospholipase C inhibitors for use in treating inflammatory disorders

(76) Inventors: Bharat Lagu, Hillsborough, NJ (US); Meng Pan, Branchburg, NJ (US); Kenneth Rupert, South Orange, NJ (US); Michael Wachter, Bloomsbury, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/815,017

(22) Filed: Mar. 31, 2004

Related U.S. Application Data
(60) Provisional application No. 60/458,938, filed on Mar. 31, 2003.

(51) Int. Cl. A61K 31/496; A61K 31/495; A61K 31/551

(52) U.S. Cl. 514/252.13; 514/255.03; 544/359; 544/392; 514/218; 540/575

(57) ABSTRACT

This invention is directed to heterocyclyl-substituted anilino phospholipase C inhibitor compounds useful in treating or ameliorating an inflammatory disorders and/or restenosis of the general formula (I):

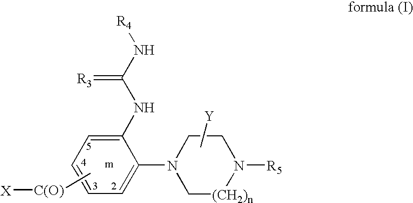

and enantiomers, diastereomers and pharmaceutically acceptable salts thereof. The present invention is further directed to pharmaceutical compositions comprising the compounds of the present invention and to methods for treating conditions affected by phospholipase modulation.

### CROSS REFERENCE TO RELATED APPLICATIONS

This present application claims benefit of U.S. Provisional Patent Application Serial No. 60/458938, filed Mar. 31, 2003, which is incorporated herein by reference in its entirety and for all purposes.

### FIELD OF THE INVENTION

This invention relates to a series of phosphoinositide-specific phospholipase C (PLC) inhibitors useful in treating or ameliorating an inflammatory disorder. More particularly, the PLC inhibitors are heterocyclyl-substituted anilino compounds useful in treating or ameliorating an inflammatory disorder.

### BACKGROUND OF THE INVENTION

Phosphoinositide-specific phospholipase C class enzymes are involved in many signaling pathways in which a cellular response (such as proliferation or secretion) is produced consequent to an extracellular stimulus. Distinct isozymes of PLC have been isolated, purified, and/or molecularly cloned from a variety of mammalian tissues. Classified on the basis of their deduced amino acid sequence, the distinct types of PLC isozymes have been identified as PLC-beta, PLC-gamma and PLC-delta (four distinct types of PLC isozymes were originally isolated and identified as PLC-alpha, PLC-beta, PLC-gamma and PLC-delta; the subtypes within the groups were named using Arabic numerals: PLC-β1, PLC-β2, PLC-β3 and PLC-β4 (Rhee, S. G., Suh, P., Ryu, S. & Lee, S. Y., Studies of Inositol Phosphalipid-Specific Phospholipase C, Science, 1989, 244:546-50). PLC-alpha was later determined to be in the PLC-delta class (Rhee S. G. & Choi, K. D., Regulation of Inositol Phospholipid-Specific Phospholipase C Isozymes, Journal of Biological Chemistry, 1992, 267:12393-96).

The subtypes differ in their ability to hydrolyze phosphatidylinositol (PI), phosphatidylinositol-4-phosphate (PIP) or phosphatidylinositol-4,5-bisphosphate (PIP2) and in their dependence on Ca²⁺. PIP2 is the main source of phospholipid second messengers and is stored in the inner leaflet of the plasma membrane. PIP2 is derived from PI by a series of kinases. PI is synthesized in the endoplasmic reticulum and is transferred to the inner plasma membrane. PI can also be further phosphorylated by PI-4-kinase, which is membrane associated in most tissues, to give PIP. Finally, PIP can also be phosphorylated by PI(4)P-5-kinases to generate PIP2 (Rhee S. G., Regulation of Phosphoinositide-Specific Phospholipase C, Ann. Rev. Biochem., 2001, 70:221-312, Majerus, Philip W., Inositol Phosphate Biochemistry, Annual Review of Biochemistry, 1992, 61:225-50).

Recruitment and activation of leukocytes are essential components of the inflammatory response. The inflammatory response is primarily controlled by two groups of proteins known as chemokines (e.g. MCP-1 (monocyte chemotactic protein-1)) and cytokines (e.g. tumor necrosis factor-α [TNF-α] or interleukin-1 [IL-1]) (Feng L., Role of Chemokines in Inflammation and Immunoregulation, Immunol. Res., 2000, 21:203-210). Resident tissue cells secrete chemokines and cytokines following tissue injury and/or the detection of the presence of an infectious agent (Gerard C., Rolling B., Chemokines and Disease, Nat. Immunol., 2000, 2:108-115).

Several cytokines (e.g., IL-1 and TNF-α) stimulate vascular endothelial cells to upregulate their expression of adhesion molecules for circulating leukocytes, while chemokines direct the movement of the leukocytes through the endothelial barrier to the site of inflammation and activate such cells once they have migrated into the lesion (Keane M. P., Strieter R. M., Chemokine Signaling in Inflammation, Crit. Care Med., 2000, 28:Suppl 4, N13-N26). Although inflammation plays a critical role in host defense to microorganisms, a poorly-regulated inflammatory response is a primary factor in the pathophysiology of several prevalent autoimmune diseases, has been implicated in the recruitment and activation of mononuclear cells in the synovial membrane in patients with rheumatoid arthritis (RA), and appears to stimulate cartilage and bone destruction. For example, the concentrations of MCP-1 (MCP-1 stimulates the upregulation of adhesion molecules on the surface of monocytes, thereby enhancing their ability to adhere to vascular endothelium, their migratory capacity and their production of superoxide anion, an essential factor in the process of killing phagocytized bacteria (Keane, 2000), MIP-1α, (macrophage inflammatory protein-1α), TNF-α and other chemokines and cytokines are increased in the inflamed joints of patients with RA, with higher levels correlating with increased severity of the disease in both man and experimental animals (Ellingsen T., et al, Plasma MCP-1 is a Marker for Joint Inflammation in Rheumatoid Arthritis, J. Rheumatol., 2001, 28:41-46; Hjelmstrom P., et al, Lymphoid Tissue Homing Chemokines are Expressed in Chronic Inflammation, Am. J. Pathol., 2000, 156:1133-1138; and, Kasama T., et al, Interleukin-10 Expression and Chemokine Regulation During the Evolution of Murine Type ii Collagen-Induced Arthritis, J. Clin. Invest., 1995, 95:2868-2876).

Chemokines also appear to be important mediators in multiple sclerosis (MS). Chemokine concentrations are elevated in the CSF (cerebrospinal fluid) of MS patients, and central nervous system T-cells in MS patients are highly enriched for certain chemokine receptors (Sorensen T. L., et al, Expression of Specific Chemokines and Chemokine Receptors in the Central Nervous System of Multiple Sclerosis Patients, J. Clin. Invest., 1999, 103:807-815). Mice deficient in MCP-1 or CCR2 (the cell-surface receptor for MCP-1) are resistant to the development of experimental autoimmune encephalomyelitis (EAE), a well-characterized animal model of MS (Fife B. T., et al, CC Chemokine Receptor 2 is Critical for Induction of Experimental Autoimnmune Encephalomyelitis, J. Exp. Med., 2002, 192:899-905; and Huang D., et al, Absence of Monocyte Chemoattractant-1 in Mice Leads to Decreased Local Macrophage Recruitment and Antigen-Specific T Helper Cell Type 1 Immune Response in Experimental Allergic Encephalomyelitis, J. Exp. Med., 2000, 193:713-725).

Many chemokines (eg interleukin-8 [IL-8]) interact with cell-surface receptors to stimulate PLCP2 via receptor-linked G-proteins (guanine-nucleotide binding proteins) (Kriz D., et al, Ciba Found, Symp., 1990, 150:112-117). Activation of PLC-β2 by the receptor-linked G-protein catalyzes the hydrolysis of PIP2 to release the second messengers 1,2-diacylglycerol (DAG) and 1,4,5-inositol trisphosphate (IP3). IP3 stimulates intracellular Ca²⁺ release, while hydrophobic DAG remains in the plasma membrane where it mediates the activation of members of the protein kinase C (“PKC”) family. PLC-β2 is found primarily in hematopoietic cells and can be activated by both the G, subunits of the G_{q} class and by the βγ subunits generated by a number of different G-proteins (Park D., et al, Cloning, Sequencing, Expression and G_{q}-Independent Activation of Phospholipase C-β2, J. Biol. Chem., 1992, 267:16048-16055).

Cotransfection experiments in COS-7 and HEK 293 cells demonstrate clearly that PLC-β2 functions downstream of several chemokine receptors (Wu D., Roles of Phospholipid Signaling in Chemoattractant-Induced Responses, J. Cell Sci., 2000, 113:2935-2940; Huping J., et al, Role of Phospholipase C-β2 in Chemoattractant-Elicited Responses, Proc. Natl. Acad. Sci. (USA), 1997, 94:7971-7975).

For example, experiments with cells expressing transfected receptors for complement component C5a, fMet-Leu-Phe (fLP) (Sigma, catalog no. F-3506), IL-8 or MCP-1 have shown that each of these receptors activates PLC-β2 through a pertussis toxin (PTx)-sensitive mechanism to release βγ subunits from the Gᵢ class of heterotrimeric G-proteins (Jiang H, et al, Pertussis Toxin-Sensitive Activation of Phospholipase C by the C5a and fMet-Leu-Phe Receptors, J. Biol. Chem., 1996, 271:13430-13434). Additional evidence for the involvement of PLC-,2 in signaling through chemokine receptors comes from experiments in knockout (KO) mice deficient in expression of the PLC-β2 protein. Although hematopoeisis is not affected in these mice, cells from the mice have decreased responsiveness to chemokines as measured by Ca²⁺ fluxes, generation of inositol phosphates, upregulation of adhesion molecules, phosphorylation of MAP kinases and production of superoxide anion (Wu D., 2000; Huping J., 1997). Surprisingly, however, leukocytes from those mice were reported to have normal or even enhanced chemotactic responses to various chemokines (Park D., 2000; Wu D., 2000; Huping J., 1997). Inhibitors of PLC-β2 enzymatic activity inhibit chemotactic responses to various chemotactic factors, suggesting that a compensatory mechanism may exist in the PLC-β2 KO mice which overcomes the congenital absence of the enzyme to allow normal or enhanced migratory responsiveness to chemokines (Park D., 2000; Wu D., 2000; Huping J., 1997).

References to a number of substituted piperazine and piperidine compounds include those disclosing use as an inhibitor of the NHE1 isoform of the sodium/hydrogen exchanger (Lorrain, J., et al; Pharmacological Profile of SL 591227, A Novel Inhibitor of the Sodium/Hydrogen Exchanger, Brit. J. Pharm., 2000, 131:1188-1194), as platelet aggregation inhibitors (acting as fibrinogen receptor antagonists) (U.S. Pat. No. 5,795,893), as tachykinin receptor antagonists (U.S. Pat. No. 5,607,936), as 5HT2C antagonists (U.S. Pat. No. 5,972,937), as 5HT1D receptor antagonists (U.S. Pat. No. 5,905,080), as enzyme acyl coenzyme A: cholesterol acyltransferase inhibitors (U.S. Pat. No. 5,185,358), as protein isoprenyl tranferase (such as protein famesyltransferase and protein geranylgeranyltransferase) inhibitors (U.S. Pat. No. 6,310,095), as cardiovascular agents (U.S. Pat. No. 5,547,966) and as antiviral agents (European Patent EP0548798). PCT application WO 93/30322 discloses thiourea compounds for treating AIDS and/or HIV.

The PLC class of enzymes play important roles in inflammatory responses. Therefore, inhibitors of PLC may be useful in treating or ameliorating inflammatory disorders. The present invention provides novel heterocyclyl-substituted anilino compounds which function as PLC inhibitors, thereby providing a means for the treatment and/or amelioration of disorders and conditions mediated by PLC-β2, including inflammatory and related disorders.

### SUMMARY OF THE INVENTION

An embodiment of the present invention includes a method for treating or ameliorating disorders and conditions mediated by PLC-β2, including inflammatory disorders in a subject in need thereof comprising administering to the subject a therapeutically effective amount of a compound of formula (I).

### DETAILED DESCRIPTION OF THE INVENTION

The present invention provides heterocyclyl-substituted anilino compounds useful for the treatment of disorders and conditions mediated by PLC-β2.

In particular, the heterocyclyl-substituted anilino compounds of the present invention are of the general formula (I):

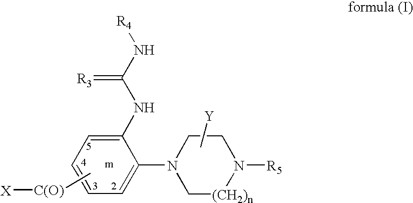

and enantiomers, diastereomers and pharmaceutically acceptable salts thereof, wherein:

X—C(O)— is a substituent moiety having a variable position “m”, wherein “m” represents a carbon atom number corresponding to a point of attachment for the X—C(O)— substituent moiety on the anilino ring of formula (I);

X is selected from the group consisting of

(i) R₁—NH— (amino optionally substituted with R₁); and,

(ii) a heterocyclyl ring optionally substituted with R₂, said heterocyclyl ring having at least one nitrogen atom member, wherein the nitrogen atom member forms the point of attachment for said heterocyclyl ring on the —C(O)— portion of X—C(O)—;

R₁ and R₂ are independently selected from the groupconsisting of hydrogen and C₁₋₈alkyl, wherein C₁₋₈alkyl is optionally substituted with one or more substituents independently selected from the group consisting of amino, mono(C₁₋₈)alkylamino, di(C₁₋₈)alkylamino, cyano, halogen, hydroxy, nitro and carboxyl;

R₃ is selected from the group consisting of O and S;

R4 is selected from the group consisting of

(a) C₁₋₈alkyl optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(b) carbonyl(C₁₋₈)alkyl, wherein the C₁₋₈alkyl portion of the carbonyl(C₁₋₈)alkyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(c) carbonyl(C₂₋₈)alkenyl, wherein the C₂₋₈alkenyl portion of the carbonyl(C₂₋₈)alkenyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(d) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(e) benzofused dioxolyl;

(f) benzofused dioxinyl;

(g) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

R₅ is one substituent selected from the group consisting of

(i) C₁₋₈alkyl optionally substituted with one or more substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,

wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₈alkyl, and optionally and independently substituted on a carbon atom with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(j) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(k) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

Y is one or more optionally present C₁₋₈alkyl substituents optionally substituted with one or more substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;

m is an integer from 2 to 5 which represents the carbon atom number corresponding to the point of attachment for the X—C(O)— substituent moiety on the anilino ring of formula (I); and, n is an integer from 1 to 2.

In an embodiment of the present invention are compounds of the formula (la):

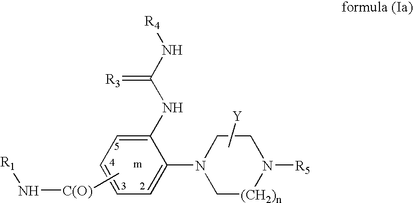

and enantiomers, diastereomers and pharmaceutically acceptable salts thereof, wherein:

R₁—NH—C(O)— is a substituent moiety having a variable position “m”, wherein “m” represents a carbon atom number corresponding to a point of attachment for the R₁—NH—C(O)— substituent moiety on the anilino ring of formula (Ia);

R₁ is selected from the group consisting of hydrogen and C₁₋₈alkyl, wherein C₁₋₈alkyl is optionally substituted with one or more substituents independently selected from the group consisting of amino, mono(C₁₋₈)alkylamino, di(C₁₋₈)alkylamino, cyano, halogen, hydroxy, nitro and carboxyl;

R₃ is selected from the group consisting of O and S;

R4 is selected from the group consisting of

(a) C₁₋₈alkyl optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(b) carbonyl(C₁₋₈)alkyl, wherein the C₁₋₈alkyl portion of the carbonyl(C₁₋₈)alkyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(c) carbonyl(C₂₋₈)alkenyl, wherein the C₂₋₈alkenyl portion of the carbonyl(C₂₋₈)alkenyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(d) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(e) benzofused dioxolyl;

(f) benzofused dioxinyl;

(g) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

R₅ is one substituent selected from the group consisting of

(i) C₁₋₈alkyl optionally substituted with one or more substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,

wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₈alkyl, and optionally and independently substituted on a carbon atom with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(j) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(k) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

Y is one or more optionally present C₁₋₈alkyl substituents optionally substituted with one or more substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;

m is an integer from 2 to 5 which represents the carbon atom number corresponding to the point of attachment for the R₁—NH—C(O)-substituent moiety on the anilino ring of formula (Ia); and, n is an integer from 1 to 2.

In an embodiment of the present invention are compounds of the formula (Ib):

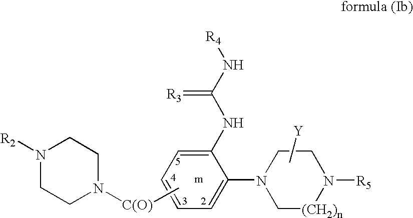

and enantiomers, diastereomers and pharmaceutically acceptable salts thereof, wherein:

(4-R₂)-1-piperazinyl-C(O)— is a substituent moiety having a variable position “m”, wherein “m” represents a carbon atom number corresponding to a point of attachment for the (4-R₂)-1-piperazinyl-C(O)— substituent moiety on the anilino ring of formula (Ib);

R₂ is selected from the group consisting of hydrogen and C₁₋₈alkyl, wherein C₁₋₈alkyl is optionally substituted with one or more substituents independently selected from the group consisting of amino, mono(C₁₋₈)alkylamino, di(C₁₋₈)alkylamino, cyano, halogen, hydroxy, nitro and carboxyl;

R₃ is selected from the group consisting of O and S; R4 is selected from the group consisting of

(a) C₁₋₈alkyl optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(b) carbonyl(C₁₋₈)alkyl, wherein the C₁₋₈alkyl portion of the carbonyl(C₁₋₈)alkyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(c) carbonyl(C₂₋₈)alkenyl, wherein the C₂₋₈alkenyl portion of the carbonyl(C₂₋₈)alkenyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(d) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(e) benzofused dioxolyl;

(f) benzofused dioxinyl;

(g) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

R₅ is one substituent selected from the group consisting of

(i) C₁₋₈alkyl optionally substituted with one or more substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,

wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₈alkyl, and optionally and independently substituted on a carbon atom with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(j) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(k) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

Y is one or more optionally present C₁₋₈alkyl substituents optionally substituted with one or more substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;

m is an integer from 2 to 5 which represents the carbon atom number corresponding to the point of attachment for the (4-R₂)-1-piperazinyl-C(O)— substituent moiety on the anilino ring of formula (Ib); and, n is an integer from 1 to 2.

In an embodiment of the present invention are compounds of the formula (Ic):

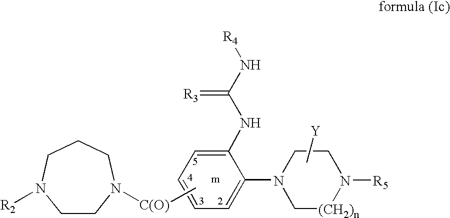

and enantiomers, diastereomers and pharmaceutically acceptable salts thereof, wherein:

(4-R₂)-hexahydro-1H-1,4-diazepin-1-yl-C(O)— is a substituent moiety having a variable position “m”, wherein “m” represents a carbon atom number corresponding to a point of attachment for the (4-R₂)-hexahydro-1H-1,4-diazepin-1-yl-C(O)— substituent moiety on the anilino ring of formula (Ic);

R₂ is selected from the group consisting of hydrogen and C₁₋₈alkyl, wherein C₁₋₈alkyl is optionally substituted with one or more substituents independently selected from the group consisting of amino, mono(C₁₋₈)alkylamino, di(C₁₋₈)alkylamino, cyano, halogen, hydroxy, nitro and carboxyl;

R₃ is selected from the group consisting of O and S;

R4 is selected from the group consisting of

(a) C₁₋₈alkyl optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(b) carbonyl(C₁₋₈)alkyl, wherein the C₁₋₈alkyl portion of the carbonyl(C₁₋₈)alkyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(c) carbonyl(C₂₋₈)alkenyl, wherein the C₂₋₈alkenyl portion of the carbonyl(C₂₋₈)alkenyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(d) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(e) benzofused dioxolyl;

(f) benzofused dioxinyl;

(g) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

R₅ is one substituent selected from the group consisting of

(i) C₁₋₈alkyl optionally substituted with one or more substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,

wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₈alkyl, and optionally and independently substituted on a carbon atom with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(j) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(k) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

Y is one or more optionally present C₁₋₈alkyl substituents optionally substituted with one or more substituents independently selected from the group consisting of amino, mono(C₁₋₄)alkylamino, di(C₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;

m is an integer from 2 to 5 which represents the carbon atom number corresponding to the point of attachment for the (4-R₂)-hexahydro-1H-1,4-diazepin-1-yl-C(O)— substituent moiety on the anilino ring of formula (Ic); and, n is an integer from 1 to 2.

In an embodiment of the present invention are compounds of formula (I), wherein when X is R₁—NH—, R₁ is hydrogen and R₄ is Cis8alkyl, then R₄ is substituted C₁₋₈alkyl.

In an embodiment of the invention are compounds of formula (Ia), wherein when the R₁—NH—C(O)— substituent moiety is NH₂—C(O)— and R₄ is C₁₋₈alkyl, then R₄ is substituted C₁₋₈alkyl.

In an embodiment of the present invention are compounds of formula (I) wherein when R₄ is unsubstituted C₁₋₈alkyl, then X is a heterocyclyl ring optionally substituted with R₂.

In an embodiment of the present invention are compounds of formulae (I) and (Ia), wherein when R₄ is optionally substituted C₁₋₈alkyl, then R₅ is C₁₋₈alkyl substituted on one or more carbon atoms with one or more optionally substituted aryl substituents.

In an embodiment of the present invention are compounds of formulae (I) and (Ia), wherein when R₄ is optionally substituted C₁₋₈alkyl, then R₅ is C₁₋₈alkyl substituted on one or two carbon atoms with one or two optionally substituted aryl substituents.

In an embodiment of the present invention are those compounds of formula (I):

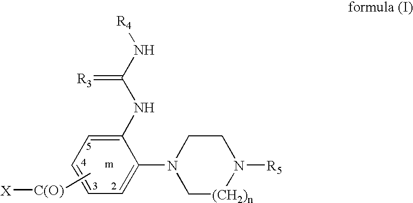

and enantiomers, diastereomers and pharmaceutically acceptable salts thereof, wherein: X—C(O)— is a substituent moiety having a variable position “m”, wherein “m” represents a carbon atom number corresponding to a point of attachment for the X—C(O)— substituent moiety on the anilino ring of formula (I);

X is selected from the group consisting of

(i) R₁—NH— wherein R₁ is selected from the group consisting of hydrogen and C₁₋₈alkyl (i.e. amino optionally substituted with one or more (C₁₋₄)alkyl substituents), wherein C₁₋₈alkyl is optionally substituted with one or more substituents independently selected from the group consisting of amino, mono(C₁₋₄)alkylamino, di(C₁₋₄)alkylamino, cyano, halogen, hydroxy, nitro and carboxyl; and,

(ii) a heterocyclyl ring selected from the group consisting of piperazinyl and hexahydro-1H-1,4-diazepinyl optionally substituted with R₂, wherein one piperazinyl and hexahydro-1H-1,4-diazepinyl ring nitrogen atom member forms the point of attachment for said ring on the —C(O)— portion of X—C(O)—; wherein R₂ is selected from the group consisting of hydrogen and C₁₋₈alkyl (i.e. wherein piperazinyl or hexahydro-1H-1,4-diazepinyl are optionally substituted with one (C₁₋₄)alkyl substituent);

R₃ is selected from the group consisting of O and S;

R₄ is selected from the group consisting of

(a) C₁₋₈alkyl optionally substituted with aryl;

(c) carbonyl(C₂₋₈)alkenyl, wherein the C₂₋₈alkenyl portion of the carbonyl(C₂₋₈)alkenyl is substituted with aryl;

(d) C₃₋₈cycloalkyl;

(e) 1,3-benzodioxol-5-yl;

(g) aryl optionally substituted with one or more substituents independently selected from the group consisting of C₁₋₈alkyl, C₁₋₈alkoxy, di(C₁₋₄)alkylamino, halogen and nitro; and,

(h) carbonyl-aryl, wherein the aryl portion of the carbonyl-aryl is optionally substituted with one halogen substituent;

R₅ is one substituent selected from the group consisting of

(i) C₁₋₈alkyl substituted with one or two substituents independently selected from the group consisting of aryl and heteroaryl, wherein said aryl is optionally substituted with one halogen substituent;

(j) C₃₋₈cycloalkyl; and,

(k) aryl optionally substituted with one C₁₋₈alkoxy substituent;

m is an integer from 2 to 5 which represents the carbon atom number corresponding to the point of attachment for the X—C(O)— substituent moiety on the anilino ring of formula (I); and, n is an integer from 1 to 2.

In an embodiment of the present invention are compounds of formula (I) wherein

R₁ is selected from the group consisting of hydrogen and C₁₋₈alkyl, wherein C₁₋₈alkyl is optionally substituted with one substituent independently selected from the group consisting of amino, mono(C₁₋₈)alkylamino, di(C₁₋₈)alkylamino and carboxyl;

R₂ is selected from the group consisting of hydrogen and C₁₋₆alkyl;

R₄ is selected from the group consisting of

(a) C₁₋₄alkyl optionally substituted with one phenyl substituent;

(c) carbonyl(C₂₋₄)alkenyl, wherein the C₂₋₄alkenyl portion of the carbonyl(C₂₋₄)alkenyl is substituted with one phenyl substituent;

(d) C₅₋₆cycloalkyl;

(e) 1,3-benzodioxol-5-yl;

(f) 2,3-dihydro-1,4-benzodioxinyl;

(g) phenyl optionally substituted with one or two substituents independently selected from the group consisting of C₁₋₄alkyl, C₁₋₄alkoxy, di(C₁₋₄)alkylamino, chlorine, fluorine and nitro; and,

(h) carbonyl-phenyl, wherein the phenyl portion of the carbonyl-phenyl is optionally substituted with one chlorine substituent;

R₅ is one substituent selected from the group consisting of

(i) C₁₋₄alkyl optionally substituted with one or two substituents independently selected from the group consisting of phenyl and pyridinyl; wherein said phenyl is optionally substituted with one chlorine or one fluorine substituent;

(j) cyclohexyl; and,

(k) fluorenyl or phenyl, wherein said phenyl is optionally substituted with one C₁₋₄alkoxy substituent;

m is an integer from 3 to 4 which represents the carbon atom number corresponding to the point of attachment for the X—C(O)— substituent moiety on the anilino ring of formula (I); and, n is 1.

In an embodiment of the present invention are compounds of formula (I) wherein X is selected from the group consisting of

(i) R₁—NH— (amino optionally substituted with RI); and,

(ii) a heterocyclyl ring optionally substituted with R₂, said heterocyclyl ring having at least one nitrogen atom member, wherein the nitrogen atom member forms the point of attachment for said heterocyclyl ring on the —C(O)— portion of X—C(O)—.

In an embodiment of the present invention are compounds of formula (I), wherein X is selected from the group consisting of

(i) R₁—NH—, wherein R₁ is selected from the group consisting of hydrogen and C₁₋₈alkyl (i.e. amino optionally substituted with one or more (C₁₋₄)alkyl substituents), wherein C₁₋₈alkyl is optionally substituted with one or two substituents independently selected from the group consisting of amino, mono(C₁₋₈)alkylamino, di(C₁₋₈)alkylamino, cyano, halogen, hydroxy, nitro and carboxyl; and,

(ii) a heterocyclyl ring selected from the group consisting of piperazinyl and hexahydro-1H-1,4-diazepinyl optionally substituted with R₂, wherein one piperazinyl and hexahydro-1H-1,4-diazepinyl ring nitrogen atom member forms the point of attachment for said ring on the —C(O)— portion of X—C(O)—; and,

wherein R₂ is selected from the group consisting of hydrogen and C₁₋₈alkyl.

In an embodiment of the present invention are those compounds of formulae (I) and (Ib), wherein X is piperazinyl optionally substituted with R₂.

In an embodiment of the present invention are compounds of formulae (I) and (Ic), wherein X is hexahydro-1,4-diazepinyl optionally substituted with R₂.

In an embodiment of the present invention are compounds of formula (I), wherein R₁ and R₂ are independently selected from the group consisting of hydrogen and C₁₋₈alkyl, wherein C₁₋₈alkyl is optionally substituted with one, two or three substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I), wherein R₁ and R₂ are independently selected from the group consisting of hydrogen and C₁₋₈alkyl, wherein C₁₋₈alkyl is optionally substituted with one or two substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I), wherein R₁ and R₂ are independently selected from the group consisting of hydrogen and C₁₆alkyl, wherein C₁₋₆alkyl is optionally substituted with one, two or three substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I), wherein R₁ and R₂ are independently selected from the group consisting of hydrogen and C₁₋₆alkyl, wherein C₁₋₆alkyl is optionally substituted with one or two substituents independently selected from the group consisting of amino, mono(C₁₋₄)alkylamino, di(C₁₋₄)alkylamino, cyano, halogen, hydroxy, nitro and carboxyl.

In an embodiment of the present invention are those compounds of formula (I), wherein R₁ and R₂ are independently selected from the group consisting of hydrogen and C₁₋₆alkyl, wherein C₁₋₆alkyl is optionally substituted with one, two or three substituents independently selected from the group consisting of amino, mono(C₁₋₄)alkylamino, di(C₁₋₄)alkylamino and carboxyl.

In an embodiment of the present invention are those compounds of formula (I), wherein R₁ and R₂ are independently selected from the group consisting of hydrogen and C₁₋₆alkyl, wherein C₁₋₆alkyl is optionally substituted with one or two substituents independently selected from the group consisting of amino, mono(C₁₋₄)alkylamino, di(C₁₋₄)alkylamino and carboxyl.

In an embodiment of the present invention are those compounds of formula (I), wherein R₁ and R₂ are independently selected from the group consisting of hydrogen and C₁₋₆alkyl, wherein C₁₋₆alkyl is optionally substituted with one substituent selected from the group consisting of amino, mono(C₄)alkylamino, di(C₁₋₄)alkylamino and carboxyl.

In an embodiment of the present invention are those compounds of formula (I), wherein R₁ and R₂ are independently selected from the group consisting of hydrogen and C₁₋₆alkyl, wherein C₁₋₆alkyl is optionally substituted with one substituent selected from the group consisting of amino, di(C₁₋₄)alkylamino and carboxyl.

In an embodiment of the present invention are compounds of formula (I) and formula (Ia), wherein R₁ is selected from the group consisting of hydrogen and C₁₋₈alkyl, wherein C₁₋₈alkyl is optionally substituted with one, two or three substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I) and formula (Ia), wherein R₁ is selected from the group consisting of hydrogen and C₁₋₈alkyl, wherein C₁₋₈alkyl is optionally substituted with one or two substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I) and formula (Ia), wherein R₁ is selected from the group consisting of hydrogen and C₁₋₆alkyl, wherein C₁₋₆alkyl is optionally substituted with one, two or three substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I) and formula (Ia), wherein R₁ is selected from the group consisting of hydrogen and C₁₋₆alkyl, wherein C₁₋₆alkyl is optionally substituted with one or two substituents independently selected from the group consisting of amino, mono(C₁₋₄)alkylamino, di(C₁₋₄)alkylamino, cyano, halogen, hydroxy, nitro and carboxyl.

In an embodiment of the present invention are those compounds of formula (I) and formula (Ia), wherein R₁ is selected from the group consisting of hydrogen and C₁₋₆alkyl, wherein C₁₋₆alkyl is optionally substituted with one, two or three substituents independently selected from the group consisting of amino, mono(C₁₋₄)alkylamino, di(C₁₋₄)alkylamino and carboxyl.

In an embodiment of the present invention are those compounds of formula (I) and formula (Ia), wherein R₁ is selected from the group consisting of hydrogen and C₁₋₆alkyl, wherein C₁₋₆alkyl is optionally substituted with one or two substituents independently selected from the group consisting of amino, mono(C₁₋₄)alkylamino, di(C₁₋₄)alkylamino and carboxyl.

In an embodiment of the present invention are those compounds of formula (I) and formula (Ia), wherein R₁ is selected from the group consisting of hydrogen and C₁₋₆alkyl, wherein C₁₋₆alkyl is optionally substituted with one substituent selected from the group consisting of amino, mono(C₁₋₄)alkylamino, di(C₁₋₄)alkylamino and carboxyl.

In an embodiment of the present invention are those compounds of formula (I) and formula (Ia), wherein R₁ is selected from the group consisting of hydrogen and C₁₋₆alkyl, wherein C₁₋₆alkyl is optionally substituted with one substituent selected from the group consisting of amino, di(C₁₋₄)alkylamino and carboxyl.

In an embodiment of the present invention are compounds of formula (I), formula (Ib) and formula (Ic) wherein R₂ is selected from the group consisting of hydrogen and C₁₋₈alkyl, wherein C₁₋₈alkyl is optionally substituted with one, two or three substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I), formula (Ib) and formula (Ic) wherein R₂ is selected from the group consisting of hydrogen and C₁₋₈alkyl, wherein C₁₋₈alkyl is optionally substituted with one or two substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I) and formula (Ia), wherein R₂ is selected from the group consisting of hydrogen and C₁₋₆alkyl, wherein C₁₋₆alkyl is optionally substituted with one, two or three substituents independently selected from the group consisting of amino, mono(C₁₋₄)alkylamino, di(C₁₋₄)alkylamino, cyano, halogen, hydroxy, nitro and carboxyl.

In an embodiment of the present invention are compounds of formula (I) and formula (Ia), wherein R₂ is selected from the group consisting of hydrogen and C₁₋₆alkyl, wherein C₁₋₆alkyl is optionally substituted with one or two substituents independently selected from the group consisting of amino, mono(C₁₋₄)alkylamino, di(C₁₋₄)alkylamino, cyano, halogen, hydroxy, nitro and carboxyl.

In an embodiment of the present invention are those compounds of formula (I), formula (Ib) and formula (Ic), wherein R₂ is selected from the group consisting of hydrogen and C₁₋₆alkyl, wherein C₁₋₆alkyl is optionally substituted with one, two or three substituents independently selected from the group consisting of amino, mono(C₁₋₄)alkylamino, di(C₁₋₄)alkylamino and carboxyl.

In an embodiment of the present invention are those compounds of formula (I), formula (Ib) and formula (Ic), wherein R₂ is selected from the group consisting of hydrogen and C₁₋₆alkyl, wherein C₁₋₆alkyl is optionally substituted with one or two substituents independently selected from the group consisting of amino, mono(C₁₋₄)alkylamino, di(C₁₋₄)alkylamino and carboxyl.

In an embodiment of the present invention are those compounds of formula (I), formula (Ib) and formula (Ic), wherein R₂ is selected from the group consisting of hydrogen and C₁₋₆alkyl, wherein C₁₋₆alkyl is optionally substituted with one substituent selected from the group consisting of amino, mono(C₁₋₄)alkylamino, di(C₁₋₄)alkylamino and carboxyl.

In an embodiment of the present invention are those compounds of formula (I), formula (Ib) and formula (Ic) wherein R₂ is selected from the group consisting of hydrogen and C₁₋₆alkyl, wherein C₁₋₆alkyl is optionally substituted with one substituent selected from the group consisting of amino, di(C₁₋₄)alkylamino and carboxyl.

In an embodiment of the present invention are compounds of formulae (I), (Ia), (Ib) and (Ic) wherein R₃ is selected from the group consisting of O and S.

In an embodiment of the present invention are compounds of formulae (I), (Ia), (Ib) and (Ic) wherein R₄ is selected from the group consisting of

(a) C₁₋₈alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(b) carbonyl(C₁₋₈)alkyl, wherein the C₁₋₈alkyl portion of the carbonyl(C₁₋₈)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁4)alkylamino, di(C₁₋₄)alkylamino, cyano, halogen, hydroxy and nitro;

(c) carbonyl(C₂₋₈)alkenyl, wherein the C₂₋₈alkenyl portion of the carbonyl(C₂₋₈)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(d) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(e) benzofused dioxolyl;

(f) benzofused dioxinyl;

(g) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic) wherein R₄ is selected from the group consisting of

(a) C₁₋₄alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(b) carbonyl(C₁₋₄)alkyl, wherein the C₁₋₄alkyl portion of the carbonyl(C₁₋₄)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(c) carbonyl(C₂₋₄)alkenyl, wherein the C₂₋₄alkenyl portion of the carbonyl(C₂₋₄)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(d) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(e) benzofused dioxolyl;

(f) benzofused dioxinyl;

(g) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic) wherein R₄ is selected from the group consisting of

(a) C₁₋₄alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(b) carbonyl(C₁₋₄)alkyl, wherein the C₁₋₄alkyl portion of the carbonyl(C₁₋₄)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(c) carbonyl(C₂₋₄)alkenyl, wherein the C₂₋₄alkenyl portion of the carbonyl(C₂₋₄)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(d) C₃₋₈cycloalkyl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(e) benzofused dioxolyl;

(f) benzofused dioxinyl;

(g) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic) wherein R₄ is selected from the group consisting of

(a) C₁₋₄alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(c) carbonyl(C₂₋₄)alkenyl, wherein the C₂₋₄alkenyl portion of the carbonyl(C₂₋₄)alkenyl is optionally substituted with one substituent selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(d) C₃₋₈cycloalkyl;

(e) benzofused dioxolyl;

(f) benzofused dioxinyl;

(g) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic) wherein R₄ is selected from the group consisting of

(a) C₁₋₄alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(c) carbonyl(C₂₋₄)alkenyl, wherein the C₂₋₄alkenyl portion of the carbonyl(C₂₋₄)alkenyl is optionally substituted with one aryl substituent, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(d) C₃₋₈cycloalkyl;

(e) benzofused dioxolyl;

(f) benzofused dioxinyl;

(g) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and,

(h) carbonyl-aryl, wherein the aryl portion of the carbonyl-aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic) wherein R₄ is selected from the group consisting of

(a) C₁₋₄alkyl optionally substituted with one or two substituents independently selected from the group consisting of amino, mono(C₁₋₄)alkylamino, di(C₁₋₄)alkylamino and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(c) carbonyl(C₂₋₄)alkenyl, wherein the C₂₋₄alkenyl portion of the carbonyl(C₂₋₄)alkenyl is optionally substituted with one aryl substituent, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(d) C₃₋₈cycloalkyl;

(e) benzofused dioxolyl;

(f) benzofused dioxinyl;

(g) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and,

(h) carbonyl-phenyl, wherein the phenyl portion of the carbonyl-phenyl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic) wherein R₄ is selected from the group consisting of

(a) C₁₋₄alkyl optionally substituted with one substituent selected from the group consisting of amino, mono(C₁₋₄)alkylamino and di(C₁₋₄)alkylamino and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(c) carbonyl(C₂₋₄)alkenyl, wherein the C₂₋₄alkenyl portion of the carbonyl(C₂₋₄)alkenyl is optionally substituted with one aryl substituent, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(d) C₃₋₈cycloalkyl;

(e) benzofused dioxolyl;

(f) benzofused dioxinyl;

(g) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and,

(h) carbonyl-phenyl, wherein the phenyl portion of the carbonyl-phenyl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic) wherein R₄ is selected from the group consisting of

(a) C₁₋₄alkyl optionally substituted with one aryl substituent, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(c) carbonyl(C₂₋₄)alkenyl, wherein the C₂₋₄alkenyl portion of the carbonyl(C₂₋₄)alkenyl is substituted with one phenyl substituent, wherein said phenyl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(d) C₃-₈cycloalkyl;

(e) benzofused dioxolyl;

(f) benzofused dioxinyl;

(g) aryl optionally substituted with one or two substituents independently selected from the group consisting of C₁₋₄alkyl, C₁₋₄alkoxy, mono(C₁₋₄)alkylamino, di(C₁₋₄)alkylamino, halogen and nitro; and,

(h) carbonyl-phenyl, wherein the phenyl portion of the carbonyl-phenyl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic) wherein R₄ is selected from the group consisting of

(a) C₁₋₄alkyl optionally substituted with one phenyl substituent, wherein said phenyl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(c) carbonyl(C₂₋₄)alkenyl, wherein the C₂₋₄alkenyl portion of the carbonyl(C₂₋₄)alkenyl is substituted with one phenyl substituent;

(d) C₃₋₈cycloalkyl;

(e) benzofused dioxolyl;

(f) benzofused dioxinyl;

(g) phenyl optionally substituted with one or two substituents independently selected from the group consisting of C₁₋₄alkyl, C₁₋₄alkoxy, di(C₁₋₄)alkylamino, halogen and nitro; and,

(h) carbonyl-phenyl, wherein the phenyl portion of the carbonyl-phenyl is optionally substituted with one halogen substituent.

In an embodiment of the present invention are those compounds of formulae (I), (Ia), (Ib) and (Ic) wherein R₄ is selected from the group consisting of

(a) C₁₋₄alkyl optionally substituted with one phenyl substituent;

(c) carbonyl(C₂₋₄)alkenyl, wherein the C₂₋₄alkenyl portion of the carbonyl(C₂₋₄)alkenyl is substituted with one phenyl substituent;

(d) C₅₋₆cycloalkyl;

(e) 1,3-benzodioxol-5-yl;

(f) 2,3-dihydro-1,4-benzodioxinyl;

(g) phenyl optionally substituted with one or two substituents independently selected from the group consisting of C₁₋₄alkyl, C₁₋₄alkoxy, di(C₁₋₄)alkylamino, chlorine, fluorine and nitro; and,

(h) carbonyl-phenyl, wherein the phenyl portion of the carbonyl-phenyl is optionally substituted with one chlorine substituent.

In an embodiment of the present invention are compounds of formulae (I), (Ia), (Ib) and (Ic), wherein R₄(a) is C₁₋₈alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(a) is C₁₋₄alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(a) is C₁₋₄alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(a) is C₁₋₄alkyl optionally substituted with one or two substituents independently selected from the group consisting of amino, mono(C₁₋₄)alkylamino, di(C₁₋₄)alkylamino and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(a) is C₁₋₄alkyl optionally substituted with one substituent selected from the group consisting of amino, mono(C₁₋₄)alkylamino, di(C₁₋₄)alkylamino and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(a) is C₁₋₄alkyl optionally substituted with one aryl substituent, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(a) is C₁₋₄alkyl optionally substituted with one phenyl substituent, wherein said phenyl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(a) is C₁₋₄alkyl substituted with one phenyl substituent.

In an embodiment of the present invention are compounds of formulae (I), (Ia), (Ib) and (Ic), wherein R₄(b) is carbonyl(C₁₋₈)alkyl, wherein the C₁₋₈alkyl portion of the carbonyl(C₁₋₈)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(b) is carbonyl(C₁₋₄)alkyl, wherein the C₁₋₄alkyl portion of the carbonyl(C₁₋₄)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(b) is carbonyl(C₁₋₄)alkyl, wherein the C₁₋₄alkyl portion of the carbonyl(C₁₋₄)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(c) is carbonyl(C₂₋₈)alkenyl, wherein the C₂₋₈alkenyl portion of the carbonyl(C₂₋₈)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(c) is carbonyl(C₂₋₄)alkenyl, wherein the C₂₋₄alkenyl portion of the carbonyl(C₂₋₄)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(c) is carbonyl(C₂₋₄)alkenyl, wherein the C₂₋₄alkenyl portion of the carbonyl(C₂₋₄)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(c) is carbonyl(C₂₋₄)alkenyl, wherein the C₂₋₄alkenyl portion of the carbonyl(C₂₋₄)alkenyl is optionally substituted with one substituent selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(c) is carbonyl(C₂₋₄)alkenyl, wherein the C₂₋₄alkenyl portion of the carbonyl(C₂₋₄)alkenyl is optionally substituted with one aryl substituent, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(c) is carbonyl(C₂₋₄)alkenyl, wherein the C₂₋₄alkenyl portion of the carbonyl(C₂₋₄)alkenyl is substituted with one phenyl substituent, wherein said phenyl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(c) is carbonyl(C₂₋₄)alkenyl, wherein the C₂₋₄alkenyl portion of the carbonyl(C₂₋₄)alkenyl is substituted with one phenyl substituent.

In an embodiment of the present invention are compounds of formulae (I), (Ia), (Ib) and (Ic), wherein R₄(d) is C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; or,

In an embodiment of the present invention are compounds of formulae (I), (Ia), (Ib) and (Ic), wherein R₄(d) is C₃₋₈cycloalkyl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein P4(d) is C₃₋₈cycloalkyl.

In an embodiment of the present invention are those compounds of formulae (I), (Ia), (Ib) and (Ic), wherein R₄(d) is C₅₋₆cycloalkyl.

In an embodiment of the present invention are those compounds of formulae (I), (Ia), (Ib) and (Ic), wherein R₄(e) is benzofused dioxolyl.

In an embodiment of the present invention are those compounds of formulae (I), (Ia), (Ib) and (Ic), wherein R₄(e) is 1,3-benzodioxolyl.

In an embodiment of the present invention are those compounds of formulae (I), (Ia), (Ib) and (Ic), wherein R₄(f) is benzofused dioxinyl.

In an embodiment of the present invention are those compounds of formulae (I), (Ia), (Ib) and (Ic), wherein R₄(f) is 2,3-dihydro-1,4-benzodioxinyl.

In an embodiment of the present invention are compounds of formulae (I), (Ia), (Ib) and (Ic), wherein R₄(g) is aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(g) is aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(g) is aryl optionally substituted with one or two substituents independently selected from the group consisting of C₁₋₄alkyl, C₁₋₄alkoxy, mono(C₁₋₄)alkylamino, di(C₁₋₄)alkylamino, halogen and nitro.

In an embodiment of the present invention are those compounds of formulae (I), (Ia), (Ib) and (Ic), wherein R₄(g) is phenyl optionally substituted with one or two substituents independently selected from the group consisting of C₁₋₄alkyl, C₁₋₄alkoxy, di(C₁₋₄)alkylamino, halogen and nitro.

In an embodiment of the present invention are those compounds of formulae (I), (Ia), (Ib) and (Ic), wherein R₄(g) is phenyl optionally substituted with one or two substituents independently selected from the group consisting of C₁₋₄alkyl, C₁₋₄alkoxy, di(C₁₋₄)alkylamino, chlorine, fluorine and nitro.

In an embodiment of the present invention are compounds of formulae (I), (Ia), (Ib), and (Ic) wherein R₄(h) is carbonyl-aryl, wherein the aryl portion of the carbonyl-aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(h) is carbonyl-aryl, wherein the aryl portion of the carbonyl-aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₄(h) is carbonyl-phenyl, wherein the phenyl portion of the carbonyl-phenyl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(h) is carbonyl-phenyl, wherein the phenyl portion of the carbonyl-phenyl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₄(h) is carbonyl-phenyl, wherein the phenyl portion of the carbonyl-phenyl is optionally substituted with one halogen substituent.

In an embodiment of the present invention are those compounds of formulae (I), (Ia), (Ib) and (Ic), wherein R₄(h) is carbonyl-phenyl, wherein the phenyl portion of the carbonyl-phenyl is optionally substituted with one chlorine substituent.

In an embodiment of the present invention are compounds of formulae (I), (Ia), (Ib) and (Ic), wherein R₅ is one substituent selected from the group consisting of:

(i) C₁₋₈alkyl optionally substituted with one or two substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,

wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

wherein said heteroaryl is optionally substituted on a secondary amine atom with C₁₋₈alkyl, and optionally and independently substituted on one or two carbon atoms with a substituent selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(j) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(k) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅ is one substituent selected from the group consisting of:

(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl,

wherein said aryl is optionally substituted one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

wherein said heteroaryl is optionally substituted on a secondary amine atom with C₁₋₈alkyl, and optionally and independently substituted on one or two carbon atoms with a substituent selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(j) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(k) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib), and (Ic) wherein R₅ is one substituent selected from the group consisting of:

(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl,

wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and,

wherein said heteroaryl is optionally substituted on a secondary amine atom with C₁₋₄alkyl, and optionally and independently substituted on one or two carbon atoms with a substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(j) C₃₋₈cycloalkyl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and,

(k) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib), and (Ic) wherein R₅ is one substituent selected from the group consisting of:

(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl, wherein said aryl is optionally substituted one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(j) C₃₋₈cycloalkyl; and,

(k) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib), and (Ic) wherein R₅ is one substituent selected from the group consisting of:

(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl,

wherein said aryl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(j) C₃₋₈cycloalkyl; and,

(k) aryl optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib), and (Ic) wherein R₅ is one substituent selected from the group consisting of:

(i) C₁₋₄alkyl optionally substituted with one or two substituents independently selected from the group consisting of aryl and heteroaryl,

wherein said aryl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(j) C₃₋₈cycloalkyl; and,

(k) aryl optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib), and (Ic) wherein R₅ is one substituent selected from the group consisting of:

(i) C₁₋₄alkyl optionally substituted with one or two substituents independently selected from the group consisting of aryl and heteroaryl,

wherein said aryl is optionally substituted with one halogen substituent;

(j) C₃₋₈cycloalkyl; and,

(k) aryl optionally substituted with one C₁₋₄alkoxy substituent.

In an embodiment of the present invention are those compounds of formulae (I), (Ia), (Ib), and (Ic) wherein R₅ is one substituent selected from the group consisting of:

(i) C₁₋₄alkyl optionally substituted with one or two substituents independently selected from the group consisting of phenyl and pyridinyl;

wherein said phenyl is optionally substituted with one halogen substituent;

(j) C₃₋₈cycloalkyl; and,

(k) aryl optionally substituted with one C₁₋₄alkoxy substituent.

In an embodiment of the present invention are those compounds of formulae (I), (Ia), (Ib), and (Ic) wherein R₅ is one substituent selected from the group consisting of:

(i) C₁₋₄alkyl optionally substituted with one or two substituents independently selected from the group consisting of phenyl and pyridinyl;

wherein said phenyl is optionally substituted with one halogen substituent;

(j) C₃₋₈cycloalkyl; and,

(k) fluorenyl or phenyl, wherein said phenyl is optionally substituted with one C₁₋₄alkoxy substituent.

In an embodiment of the present invention are those compounds of formulae (I), (Ia), (Ib), and (Ic) wherein R₅ is one substituent selected from the group consisting of:

(i) C₁₋₄alkyl optionally substituted with one or two substituents independently selected from the group consisting of phenyl and pyridinyl;

wherein said phenyl is optionally substituted with one chlorine or one fluorine substituent;

(j) cyclohexyl; and,

(k) fluorenyl or phenyl, wherein said phenyl is optionally substituted with one C₁₋₄alkoxy substituent.

In an embodiment of the present invention are compounds of formula (I) wherein R₅ is one substituent selected from the group consisting of:

(i) C₁₋₄alkyl optionally substituted with one or two substituents independently selected from the group consisting of C₁₋₈alkoxy, amino, mono(C₁₋₄)alkylamino, di(C)₄)alkylamino, cyano, halogen, hydroxy, nitro, aryl and heteroaryl,

wherein said aryl is optionally substituted with one or two substituents independently selected from the group consisting of C₁₋₈alkyl, C₁₋₈alkoxy, amino, mono(CA4)alkylamino, di(CA4)alkylamino, cyano, halogen, hydroxy and nitro; and

wherein said heteroaryl is optionally substituted on a secondary amine atom with C₁₋₈alkyl, and optionally and independently substituted on one or two carbon atoms with a substituent selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(j) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(k) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formula (I) wherein R₅ is one substituent selected from the group consisting of:

(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl,

wherein said aryl is optionally substituted one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₈alkyl, and optionally and independently substituted on one or two carbon atoms with a substituent selected from the group consisting of C₁₋₈alkyl, C₁₋₈alkoxy, amino, mono(C₁₋₄)alkylamino, di(C₁₋₄)alkylamino, cyano, halogen, hydroxy and nitro;

(j) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,

(k) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formula (I) wherein R₅ is one substituent selected from the group consisting of:

(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₄alkyl, and optionally and independently substituted on one or two carbon atoms with a substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(j) C₃₋₈cycloalkyl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and,

(k) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formula (I) wherein R₅ is one substituent selected from the group consisting of:

(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl,

wherein said aryl is optionally substituted one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(j) C₃₋₈cycloalkyl; and,

(k) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formula (I) wherein R₅ is one substituent selected from the group consisting of:

(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl,

wherein said aryl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(j) C₃₋₈cycloalkyl; and,

(k) aryl optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formula (I) wherein R₅ is one substituent selected from the group consisting of:

(i) C₁₋₄alkyl optionally substituted with one or two substituents independently selected from the group consisting of aryl and heteroaryl,

wherein said aryl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro;

(j) C₃₋₈cycloalkyl; and,

(k) aryl optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formula (I) wherein R₅ is one substituent selected from the group consisting of:

(i) C₁₋₄alkyl optionally substituted with one or two substituents independently selected from the group consisting of aryl and heteroaryl,

wherein said aryl is optionally substituted with one halogen substituent;

(j) C₃₋₈cycloalkyl; and,

(k) aryl optionally substituted with one C₁₋₄alkoxy substituent.

In an embodiment of the present invention are those compounds of formula (I) wherein R₅ is one substituent selected from the group consisting of:

(i) C₁₋₄alkyl optionally substituted with one or two substituents independently selected from the group consisting of phenyl and pyridinyl;

wherein said phenyl is optionally substituted with one halogen substituent;

(j) C₃₋₈cycloalkyl; and,

(k) aryl optionally substituted with one C₁₋₄alkoxy substituent.

In an embodiment of the present invention are those compounds of formula (I) wherein R₅ is one substituent selected from the group consisting of:

(i) C₁₋₄alkyl optionally substituted with one or two substituents independently selected from the group consisting of phenyl and pyridinyl;

wherein said phenyl is optionally substituted with one halogen substituent;

(j) C₃₋₈cycloalkyl; and,

(k) fluorenyl or phenyl, wherein said phenyl is optionally substituted with one C₁₋₄alkoxy substituent.

In an embodiment of the present invention are those compounds of formula (I) wherein R₅ is one substituent selected from the group consisting of:

(i) C₁₋₄alkyl optionally substituted with one or two substituents independently selected from the group consisting of phenyl and pyridinyl;

wherein said phenyl is optionally substituted with one chlorine or one fluorine substituent;

(j) cyclohexyl; and,

(k) fluorenyl or phenyl, wherein said phenyl is optionally substituted with one C₁₋₄alkoxy substituent.

In an embodiment of the present invention are compounds of formulae (I), (Ia), (Ib) and (Ic), wherein R₅(i) is one C₁₋₈alkyl substituent optionally substituted with one or two substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,

wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₈alkyl, and optionally and independently substituted on one or two carbon atoms with a substituent selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅(i) is one C₁₋₄alkyl substituent optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl,

wherein said aryl is optionally substituted one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₈alkyl, and optionally and independently substituted on one or two carbon atoms with a substituent selected from the group consisting of C₁₋8alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅(i) is one C₁₋₄alkyl substituent optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl,

wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro; and

wherein said heteroaryl is optionally substituted on a secondary amine atom with C₁₋₄alkyl, and optionally and independently substituted on one or two carbon atoms with a substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅(i) is one C₁₋₄alkyl substituent optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl, wherein said aryl is optionally substituted one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅(i) is one C₁₋₄alkyl substituent optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl, wherein said aryl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅(i) is one C₁₋₄alkyl substituent optionally substituted with one or two substituents independently selected from the group consisting of aryl and heteroaryl, wherein said aryl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib) and (Ic), wherein R₅(i) is one C₁₋₄alkyl substituent optionally substituted with one or two substituents independently selected from the group consisting of aryl and heteroaryl; wherein said aryl is optionally substituted with one halogen substituent.

In an embodiment of the present invention are those compounds of formulae (I), (Ia), (Ib) and (Ic), wherein R₅(i) is one C₁₋₄alkyl substituent optionally substituted with one or two substituents independently selected from the group consisting of phenyl and pyridinyl; wherein said phenyl is optionally substituted with one halogen substituent.

In an embodiment of the present invention are those compounds of formulae (I), (Ia), (Ib) and (Ic), wherein R₅(i) is one C₁₋₄alkyl substituent optionally substituted with one or two substituents independently selected from the group consisting of phenyl and pyridinyl; wherein said phenyl is optionally substituted with one chlorine or one fluorine substituent.

In an embodiment of the present invention are compounds of formulae (I), (Ia), (Ib) and (Ic), wherein R₅(i) is one C₃₋₈cycloalkyl substituent optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅(i) is one C₃₋₈cycloalkyl substituent optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib), and (Ic) wherein R₅(i) is one C₃₋₈cycloalkyl substituent.

In an embodiment of the present invention are those compounds of formulae (I), (Ia), (Ib), and (Ic) wherein R₅(i) is one cyclohexyl substituent.

In an embodiment of the present invention are compounds of formulae (I), (Ia), (Ib) and (Ic), wherein R₅(k) is one aryl substituent optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅(k) is one aryl substituent optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are compounds of formulae (I), (Ia), (Ib) and (Ic), wherein R₅(k) is one aryl substituent optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

In an embodiment of the present invention are those compounds of formulae (I), (Ia), (Ib), and (Ic) wherein R₅(k) is one aryl substituent optionally substituted with one C₁₋₄alkoxy substituent.

In an embodiment of the present invention are those compounds of formulae (I), (Ia), (Ib), and (Ic) wherein R₅(k) is one fluorenyl or phenyl substituent, wherein said phenyl is optionally substituted with one C₁₋₄alkoxy substituent.

In an embodiment of the present invention are compounds of formulae (I), (Ia), (Ib) and (Ic) wherein Y is one or two optionally present C₁₋₈alkyl substituents optionally substituted with one or two substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted.

In an embodiment of the present invention are compounds of formula (I), formula (Ia), formula (Ib) and formula (Ic) wherein Y is one or two optionally present C₁₋₄alkyl substituents optionally substituted with one or two substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted.

In an embodiment of the present invention are compounds of formula (I), formula (Ia), formula (Ib) and formula (Ic) wherein Y is one or two optionally present C₁₋₄alkyl substituents optionally substituted with one or two substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted.

In an embodiment of the present invention are compounds of formula (I), formula (Ia), formula (Ib) and formula (Ic) wherein Y is one or two optionally present C₁₋₄alkyl substituents optionally substituted with one or two substituents independently selected from the group consisting of amino, mono(C₁₋₄)alkylamino, di(C₁₋₄)alkylamino.

In an embodiment of the present invention are compounds of formula (I), formula (Ia), formula (Ib) and formula (Ic) wherein Y is one or two optionally present C₁₋₄alkyl substituents optionally substituted with one or two substituents independently selected from the group consisting of cyano, halogen, hydroxy, nitro, C₃₋₈cycloalkyl, aryl and heteroaryl, wherein said C₃₋₈cycloalkyl, aryl and heteroaryl are optionally further substituted.

In an embodiment of the present invention are compounds of formula (I), formula (Ia), formula (Ib) and formula (Ic) wherein Y is one or two optionally present C₁₋₄alkyl substituents optionally substituted with one or two substituents independently selected from the group consisting of halogen, hydroxy, C₃₋₈cycloalkyl, aryl and heteroaryl, wherein said C₃₋₈cycloalkyl, aryl and heteroaryl are optionally further substituted.

In an embodiment of the present invention are compounds of formula (I), formula (Ia), formula (Ib) and formula (Ic) wherein Y is one or two optionally present C₁₋₄alkyl substituents optionally substituted with one or two substituents independently selected from the group consisting of C₃₋₈cycloalkyl, aryl and heteroaryl, wherein said C₃₋₈cycloalkyl, aryl and heteroaryl are optionally further substituted.

In an embodiment of the present invention are compounds of formula (I), formula (Ia), formula (Ib) and formula (Ic) wherein Y is absent.

Embodiments of the present invention include a compound of formula (I), wherein m is 5, as shown below:

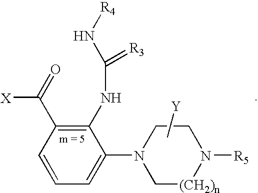

Further, embodiments of the present invention include a compound of formula (I), wherein m is 4 as shown below:

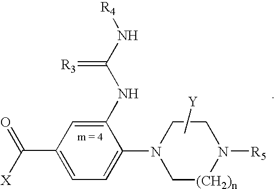

Further, embodiments of the present invention include a compound of formula (I), wherein m is 3 as shown below:

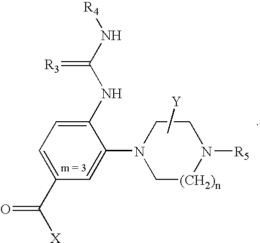

Further, embodiments of the present invention include a compound of formula (I), wherein m is 2 as shown below:

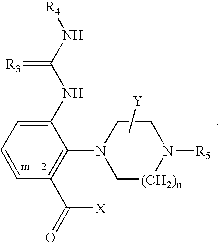

Further embodiments of the present invention include a compound of formulae (Ia), (Ib) and (Ic), wherein

m is 5; or

wherein m is 2;

or, preferably, wherein m is 3;

or, preferably, wherein m is 4.

Embodiments of the present invention include a compound of formula (I), (Ia), (Ib) and (Ic) wherein n is an integer from 1 to 2.

Embodiments of the present invention are those compounds of formula (I):

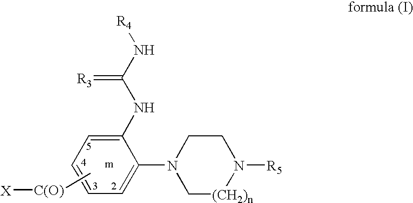

wherein X, R₃, R₄, R₅, m and n are dependently selected from the group consisting of:

| Cpd | X | m | R₃ | R₄ | n | R₅ |
|---|---|---|---|---|---|---|
| 1 | —NH₂ | 4 | O | —Ph | 1 | -2-OCH₃—Ph |
| 2 | —NH₂ | 4 | O | —Ph | 1 | —CH₂Ph |
| 3 | —NH₂ | 4 | O | —Ph | 1 | —CH(4-F—Ph)₂ |
| 4 | —NH₂ | 4 | O | -2-F—Ph | 1 | —CH(4-F—Ph)₂ |
| 5 | —NH₂ | 4 | O | -4-NO₂—Ph | 1 | —CH(Ph)₂ |
| 6 | —NH₂ | 4 | O | —CH₂Ph | 1 | —CH(Ph)₂ |
| 7 | —NH₂ | 4 | O | -3,5-(CH₃)₂—Ph | 1 | —CH(Ph)₂ |
| 8 | —NH₂ | 4 | O | —Ph | 1 | —CH(Ph)₂ |
| 9 | —NH₂ | 4 | O | —Ph | 1 | -9H-fluoren-9-yl |
| 10 | —NH₂ | 4 | O | -cyclohexyl | 1 | —CH(Ph)₂ |
| 11 | —NH₂ | 4 | O | —CH(CH₃)—Ph | 1 | —CH(Ph)₂ |
| 12 | —NH₂ | 4 | O | —(CH₂)₃CH₃ | 1 | —CH(Ph)₂ |
| 13 | —NH₂ | 4 | O | -4-F—Ph | 1 | —CH(Ph)₂ |
| 14 | —NH₂ | 4 | O | -1,3-benzodioxol- | 1 | —CH(Ph)₂ |
| | | | | 5-yl | | |
| 15 | —NH₂ | 4 | O | -2,4-(CH₃)₂—Ph | 1 | —CH(4-F—Ph)₂ |
| 16 | —NH₂ | 4 | O | —CH(CH₃)—Ph | 1 | —CH(4-F—Ph)₂ |
| 17 | —NH₂ | 4 | O | -2-OCH₃—Ph | 1 | —CH(4-F—Ph)₂ |
| 18 | —NH₂ | 4 | O | -2,4-(OCH₃)₂—Ph | 1 | —CH(4-F—Ph)₂ |
| 19 | —NH₂ | 4 | O | -4-N(CH₃)₂—Ph | 1 | —CH(4-F—Ph)₂ |
| 20 | —NH₂ | 4 | O | -4-OCH₃—Ph | 1 | —CH(4-F—Ph)₂ |
| 21 | —NH₂ | 4 | S | —CH₂Ph | 1 | —CH(4-F—Ph)₂ |
| 22 | —NH₂ | 4 | S | —Ph | 1 | —CH(4-F—Ph)₂ |
| 23 | -4-CH₃-piperazin-1-yl | 4 | O | —Ph | 1 | —CH(Ph)₂ |
| 24 | -hexahydro-1H-1,4- | 4 | O | —Ph | 1 | —CH(Ph)₂ |
| | diazepin-1-yl | | | | | |
| 25 | -hexahydro-1H-1,4- | 4 | O | -cyclohexyl | 1 | —CH(Ph)₂ |
| | diazepin-1-yl | | | | | |
| 26 | —NH—(CH₂)₂—NH₂ | 4 | O | —Ph | 1 | —CH(Ph)₂ |
| 27 | —NH—(CH₂)₂—NH₂ | 4 | O | -cyclohexyl | 1 | —CH(Ph)₂ |
| 28 | -4-CH₃-piperazin-1-yl | 4 | O | -cyclohexyl | 1 | —CH(Ph)₂ |
| 29 | —NH—(CH₂)₂—N(CH₃)₂ | 4 | O | —Ph | 1 | —CH(Ph)₂ |
| 30 | —NH—(CH₂)₂—N(CH₃)₂ | 4 | O | -cyclohexyl | 1 | —CH(Ph)₂ |
| 31 | -hexahydro-4-CH₃-1H- | 4 | O | -cyclohexyl | 1 | —CH(Ph)₂ |
| | 1,4-diazepin-1-yl | | | | | |
| 32 | —NH—CH(CO₂H)—[(S)— | 4 | O | —Ph | 1 | —CH(Ph)₂ |
| | CH₂CH(CH₃)₂] | | | | | |
| 38 | -1-piperazinyl | 4 | O | -cyclohexyl | 1 | —CH(Ph)₂ |
| 39 | -1-piperazinyl | 4 | O | —CH₂Ph | 1 | —CH(Ph)₂ |
| 40 | -1-piperazinyl | 4 | O | —Ph | 1 | —CH(Ph)₂ |
| 41 | -1-piperazinyl | 4 | O | -2,4-(CH₃)₂—Ph | 1 | —CH(Ph)₂ |
| 42 | -1-piperazinyl | 4 | O | -3,5-(CH₃)₂—Ph | 1 | —CH(Ph)₂ |
| 43 | -1-piperazinyl | 4 | O | -4-OCH₃—Ph | 1 | —CH(Ph)₂ |
| 44 | -1-piperazinyl | 4 | O | —Ph | 1 | -9H-fluoren-9-yl |
| 45 | -1-piperazinyl | 4 | O | -cyclohexyl | 1 | —CH(Ph)-(4-Cl—Ph) |
| 46 | -1-piperazinyl | 4 | O | —Ph | 1 | —CH(4-F—Ph)₂ |
| 47 | -1-piperazinyl | 4 | O | -cyclohexyl | 1 | —CH(4-F—Ph)₂ |
| 48 | -1-piperazinyl | 4 | O | —Ph | 1 | —CH(CH)₃—Ph |
| 49 | -1-piperazinyl | 4 | O | —Ph | 1 | -2-OCH₃—Ph |
| 50 | -1-piperazinyl | 4 | O | —Ph | 1 | —CH₂Ph |
| 51 | -1-piperazinyl | 4 | O | —Ph | 1 | -cyclohexyl |
| 52 | -1-piperazinyl | 4 | O | -cyclohexyl | 1 | —CH₂Ph |
| 53 | -1-piperazinyl | 4 | O | -cyclohexyl | 1 | —CH(CH₃)—Ph |
| 54 | -1-piperazinyl | 4 | O | -cyclohexyl | 1 | -cyclohexyl |
| 55 | -1-piperazinyl | 4 | O | -cyclohexyl | 1 | -2-OCH₃—Ph |
| 56 | -1-piperazinyl | 4 | O | —(CH₂)₃CH₃ | 1 | —CH(Ph)₂ |
| 57 | -1-piperazinyl | 4 | O | -2-F—Ph | 1 | —CH(Ph)₂ |
| 58 | -1-piperazinyl | 4 | O | -4-N(CH₃)₂—Ph | 1 | —CH(Ph)₂ |
| 59 | -1-piperazinyl | 4 | O | -2-OCH₃—Ph | 1 | —CH(Ph)₂ |
| 60 | -1-piperazinyl | 4 | O | —Ph | 1 | —CH(Ph)-(4-Cl—Ph) |
| 61 | -1-piperazinyl | 4 | O | —C(O)—(CH)₂—Ph | 1 | —CH(Ph)₂ |
| 62 | -1-piperazinyl | 4 | O | —C(CH₃)₃ | 1 | —CH(Ph)₂ |
| 63 | -1-piperazinyl | 4 | O | -cyclopentyl | 1 | —CH(Ph)₂ |
| 64 | -1-piperazinyl | 4 | O | —CH[(S)CH₃]—Ph | 1 | —CH(Ph)₂ |
| 65 | -1-piperazinyl | 4 | S | —CH₂Ph | 1 | —CH(Ph)₂ |
| 66 | -1-piperazinyl | 4 | O | —CH(CH₃)₂ | 1 | —CH(Ph)₂ |
| 67 | -1-piperazinyl | 4 | S | —C(O)-4-Cl—Ph | 1 | —CH(Ph)₂ |
| 77 | —NH₂ | 4 | O | —Ph | 1 | —CH(4-pyridinyl)- |
| | | | | | | (4-F—Ph) |
| 78 | —NH₂ | 4 | O | -cyclohexyl | 1 | —CH(4-pyridinyl)- |
| | | | | | | (4-F—Ph) |
| 79 | —NH₂ | 4 | O | —Ph | 2 | —CH(4-pyridinyl)- |
| | | | | | | (4-F—Ph) |
| 80 | —NH₂ | 4 | O | -cyclohexyl | 2 | —CH(4-pyridinyl)- |
| | | | | | | (4-F—Ph) |
| 81 | —NH₂ | 4 | O | —Ph | 2 | —CH(4-F—Ph)₂ |
| 82 | —NH₂ | 4 | O | -cyclohexyl | 2 | —CH(4-F—Ph)₂ |
| 89 | -1-piperazinyl | 3 | O | —Ph | 2 | —CH(4-F—Ph)₂ |
| 90 | -1-piperazinyl | 3 | O | —Ph | 1 | —CH(4-F—Ph)₂ |
| 91 | -1-piperazinyl | 3 | O | —Ph | 1 | —CH(Ph)₂ |
| 92 | -1-piperazinyl | 3 | O | -cyclohexyl | 1 | —CH(Ph)₂, and |
| 93 | —NH₂ | 3 | O | —Ph | 1 | —CH(Ph)₂. |

Embodiments of the present invention include compounds of formula (I), (Ia), (Ib) and (Ic) selected from the group consisting of:

Cpd. No. 1

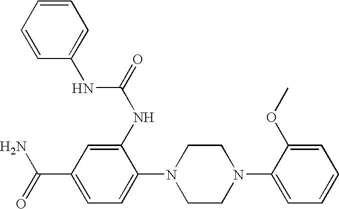

Cpd. No. 2

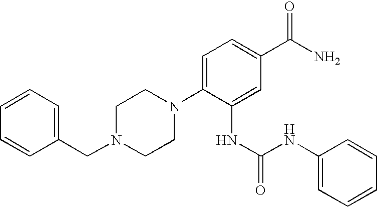

Cpd. No. 3

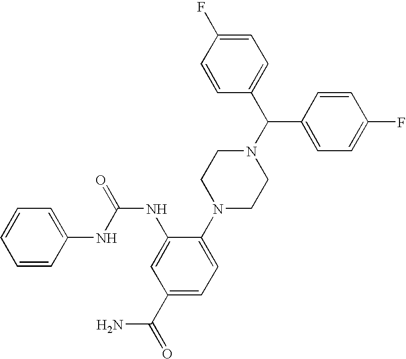

Cpd. No. 4

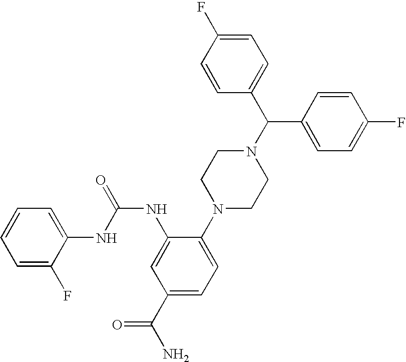

Cpd. No. 5

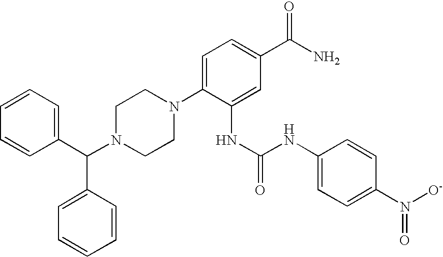

Cpd. No. 6

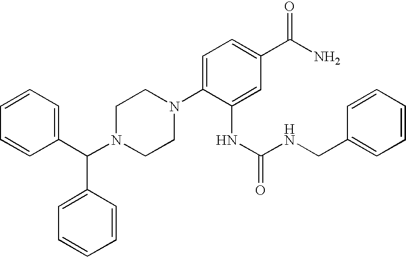

Cpd. No. 7

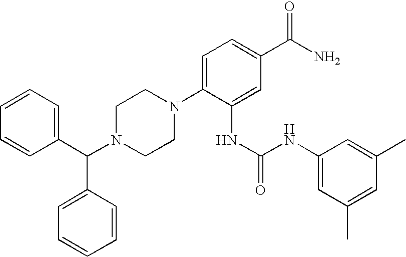

Cpd. No. 8

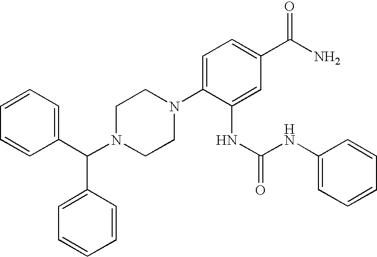

Cpd. No. 9

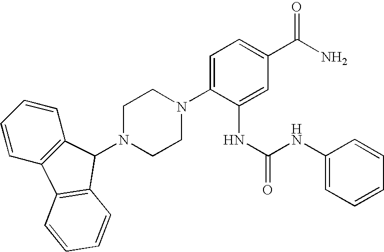

Cpd. No. 10

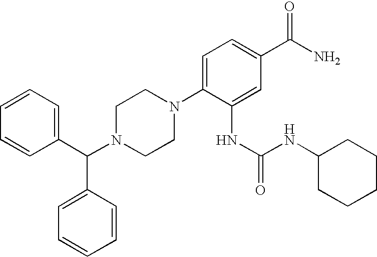

Cpd. No. 11

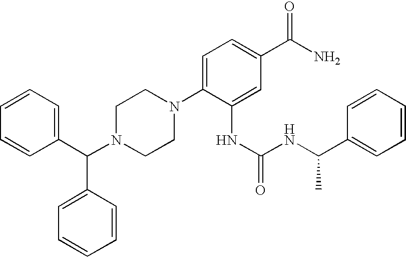

Cpd. No. 12

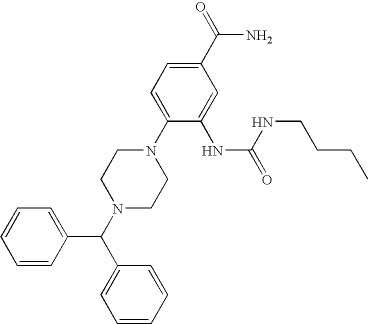

Cpd. No. 13

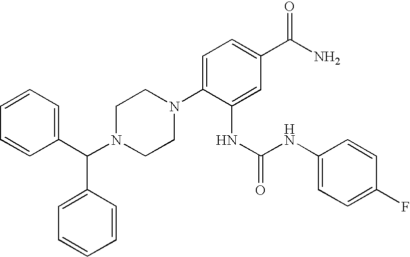

Cpd. No. 14

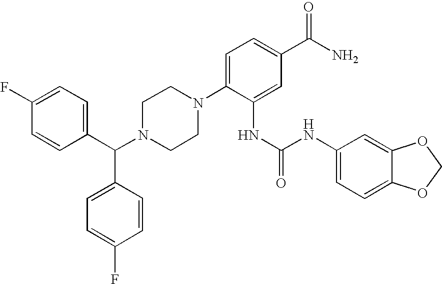

Cpd. No. 15

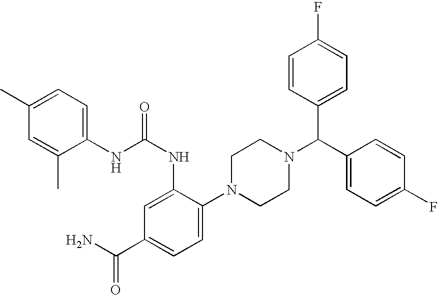

Cpd. No. 16

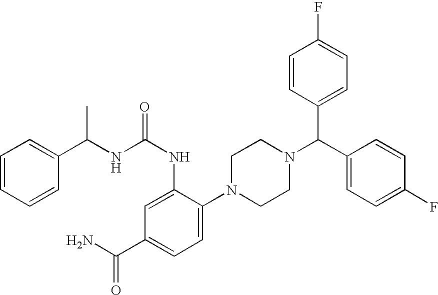

Cpd. No. 17

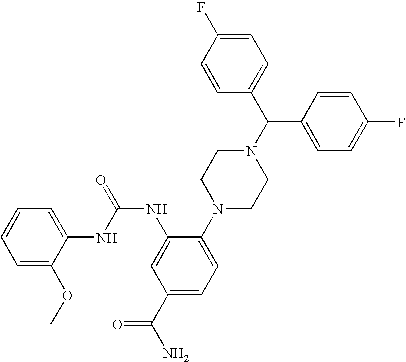

Cpd. No. 18

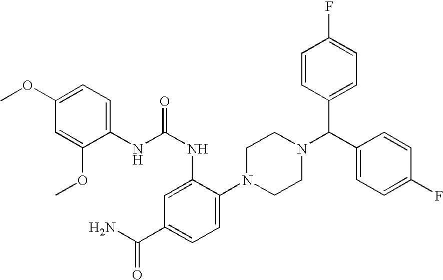

Cpd. No. 19

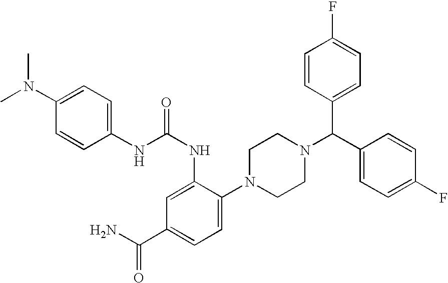

Cpd. No. 20

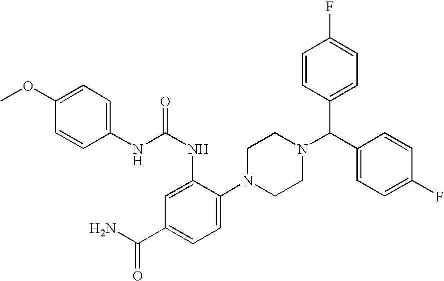

Cpd. No. 21

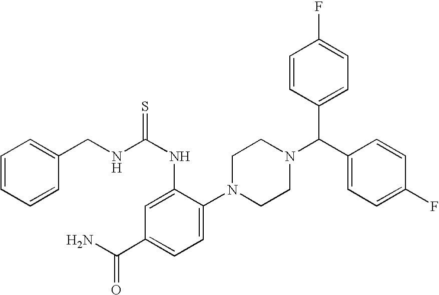

Cpd. No. 22

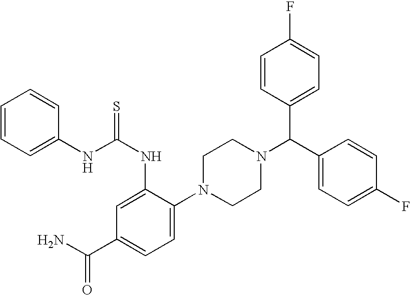

Cpd. No. 23

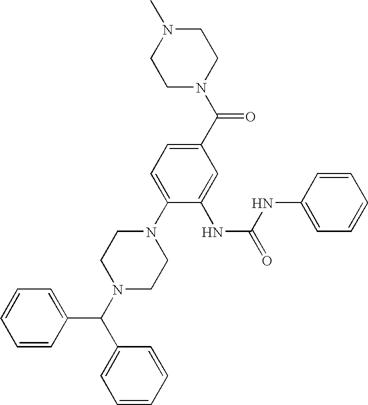

Cpd. No. 24

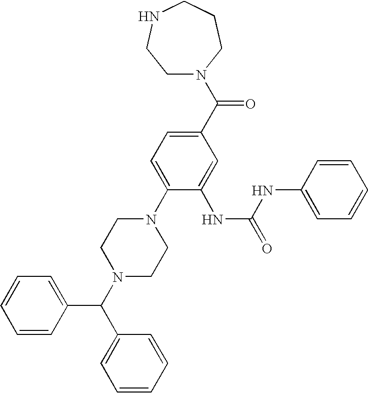

Cpd. No. 25

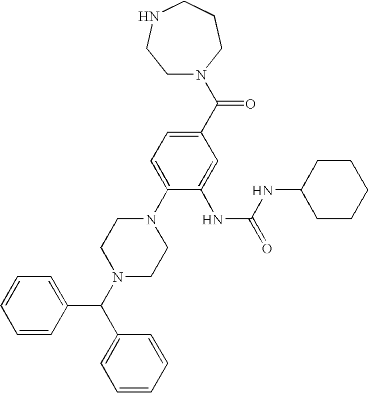

Cpd. No. 26

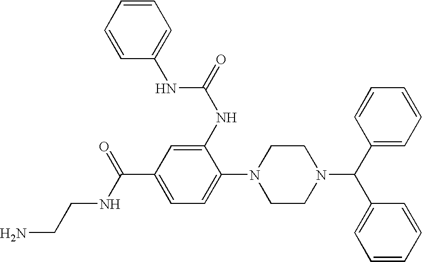

Cpd. No. 27

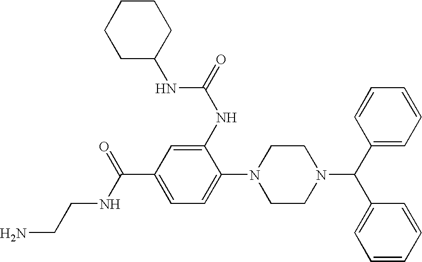

Cpd. No. 28

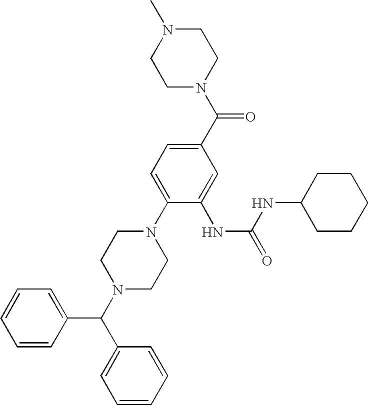

Cpd. No. 29

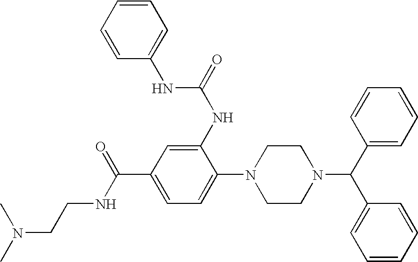

Cpd. No. 30

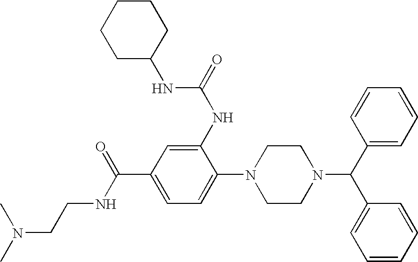

Cpd. No. 31

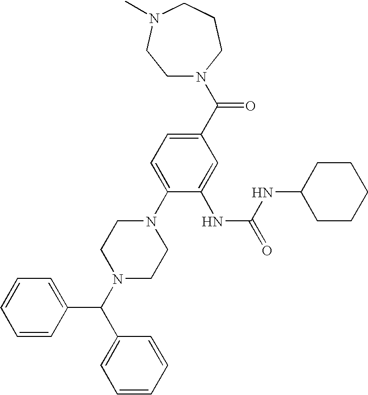

Cpd. No. 32

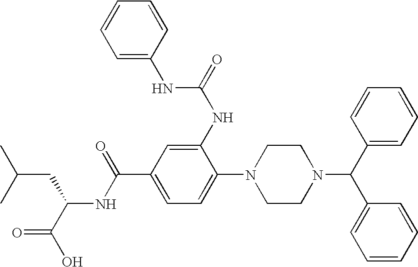

Cpd. No. 38

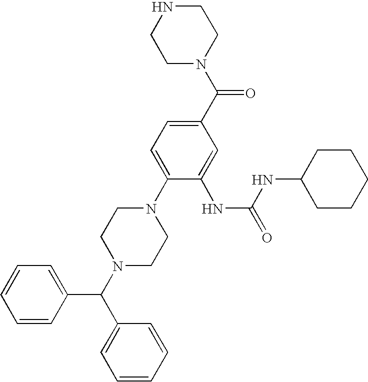

Cpd. No. 39

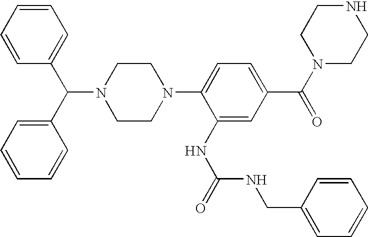

Cpd. No. 40

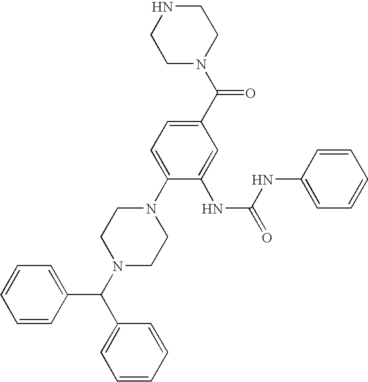

Cpd. No. 41

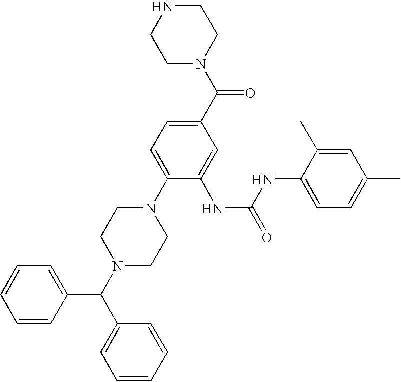

Cpd. No. 42

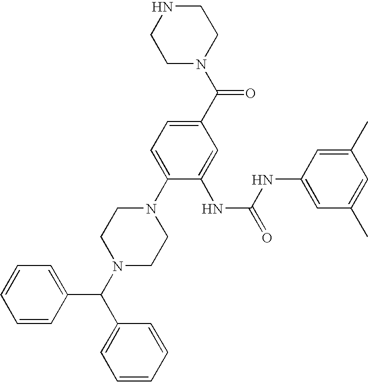

Cpd. No. 43

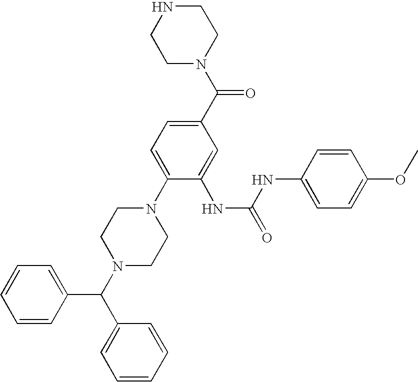

Cpd. No. 44

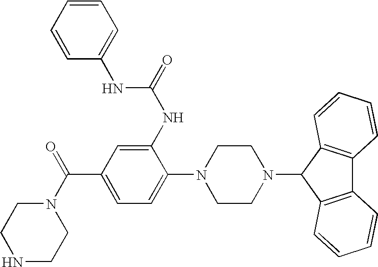

Cpd. No. 45

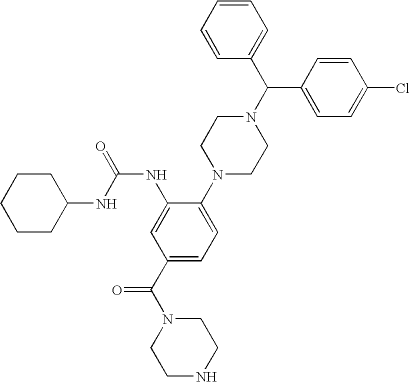

Cpd. No. 46

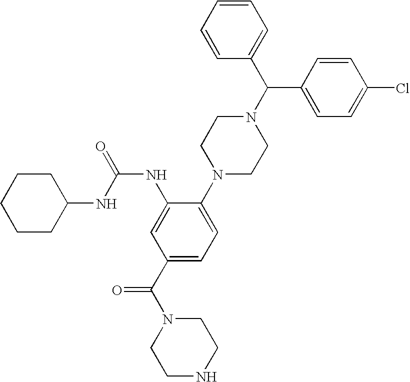

Cpd. No. 47

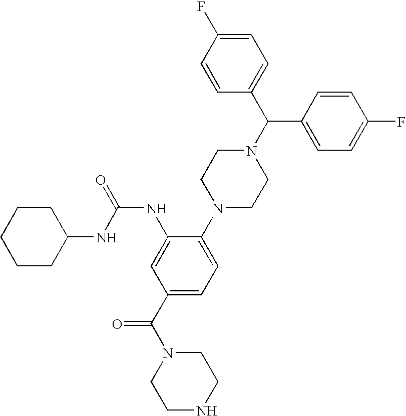

Cpd. No. 48

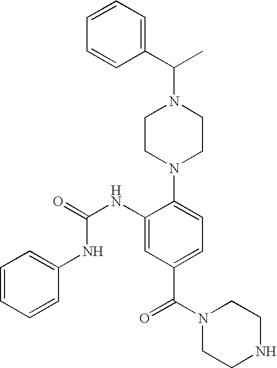

Cpd. No. 49

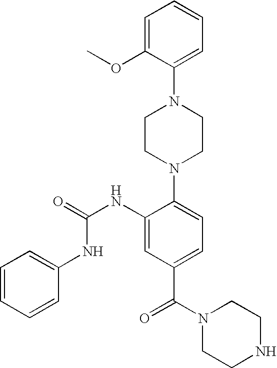

Cpd. No. 50

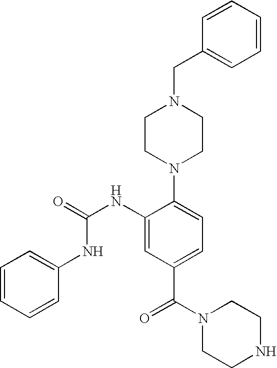

Cpd. No. 51

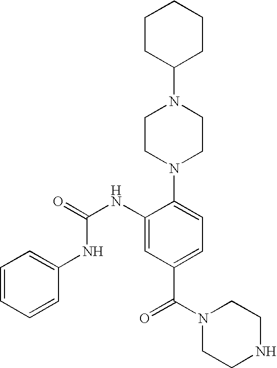

Cpd. No. 52

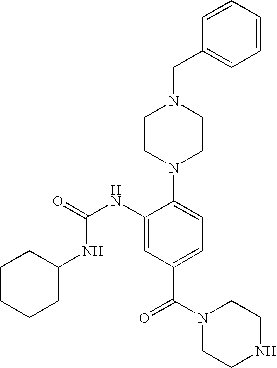

Cpd. No. 53

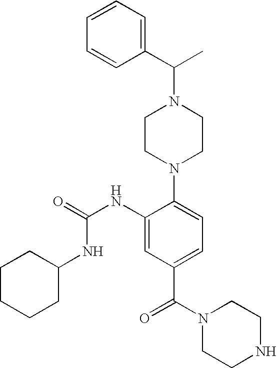

Cpd. No. 54

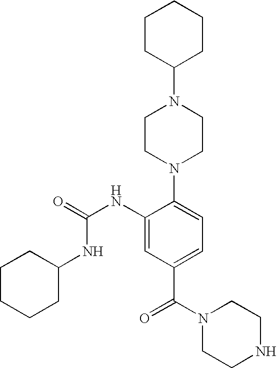

Cpd. No. 55

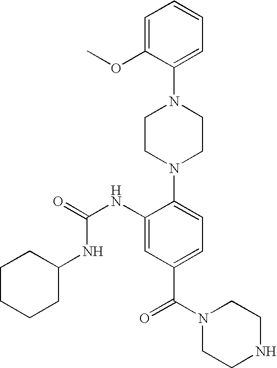

Cpd. No. 56

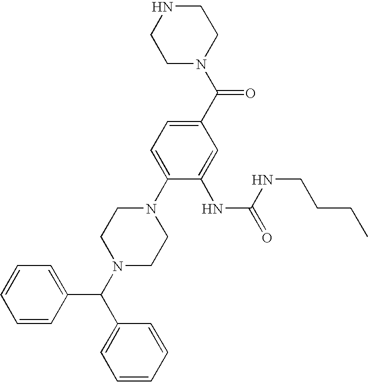

Cpd. No. 57

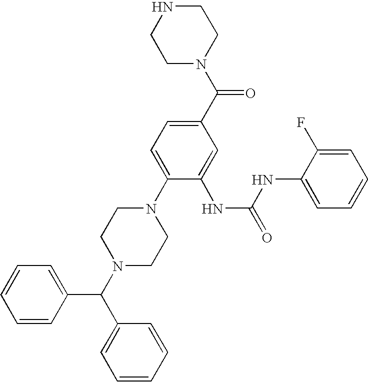

Cpd. No. 58

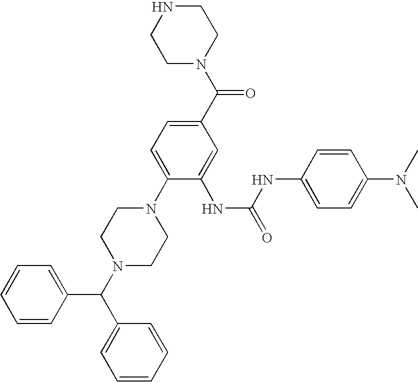

Cpd. No. 59

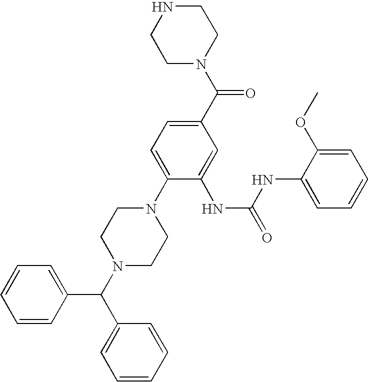

Cpd. No. 60

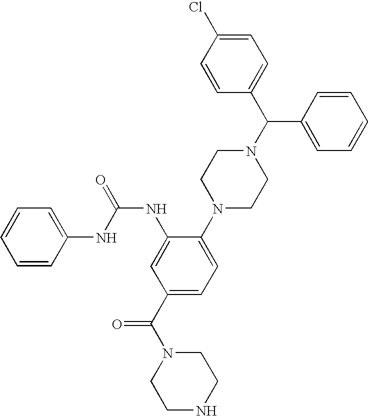

Cpd. No. 61

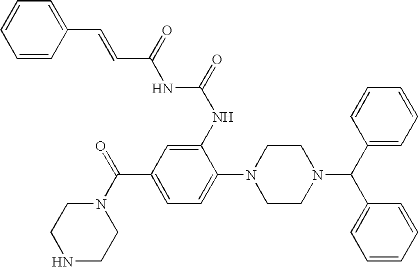

Cpd. No. 62

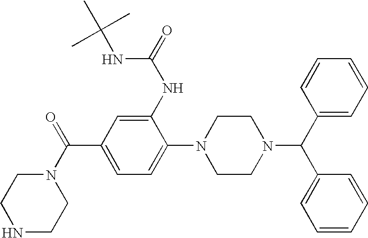

Cpd. No. 63

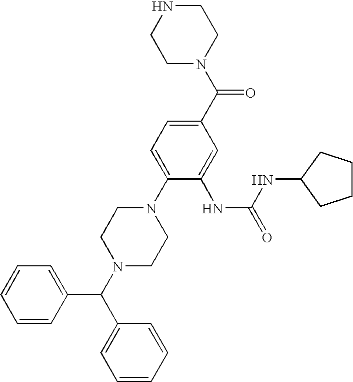

Cpd. No. 64

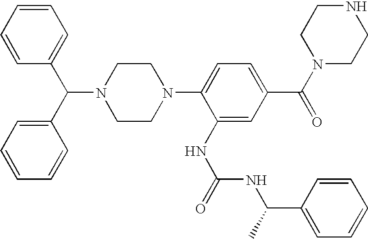

Cpd. No. 65

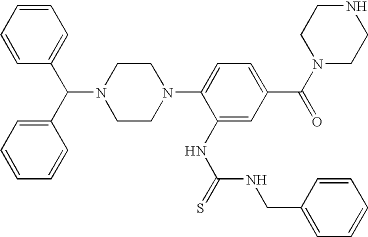

Cpd. No. 66

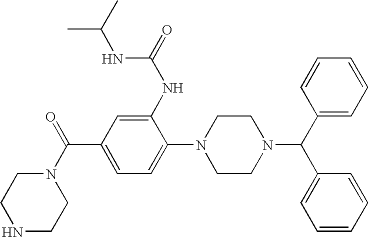

Cpd. No. 67

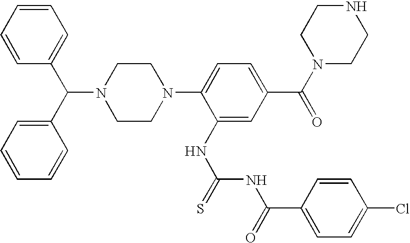

Cpd. No. 77

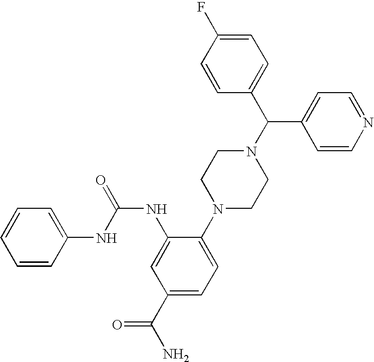

Cpd. No. 78

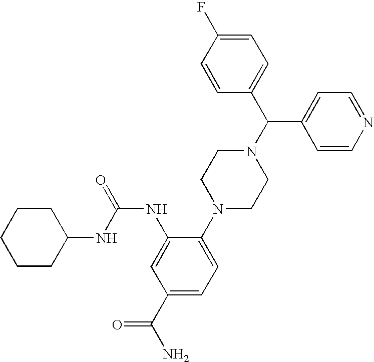

Cpd. No. 79

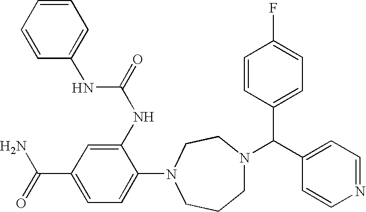

Cpd. No. 80

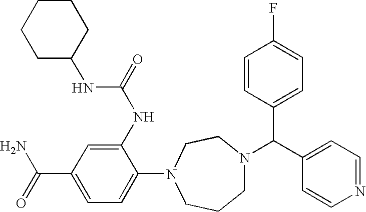

Cpd. No. 81

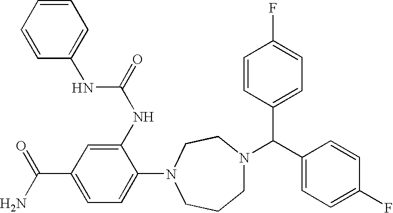

Cpd. No. 82

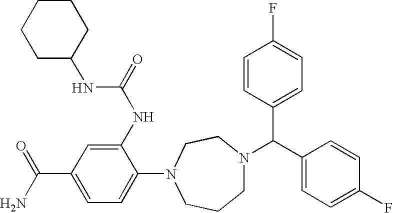

Cpd. No. 89

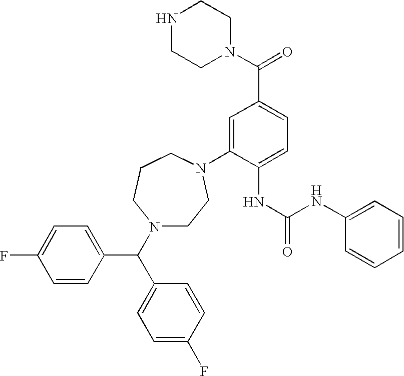

Cpd. No. 90

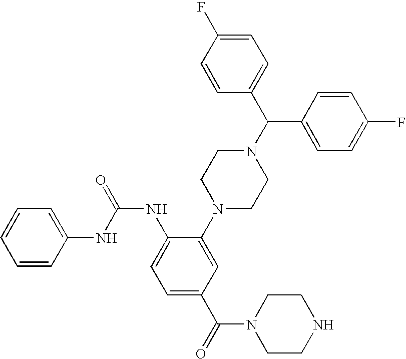

Cpd. No. 91

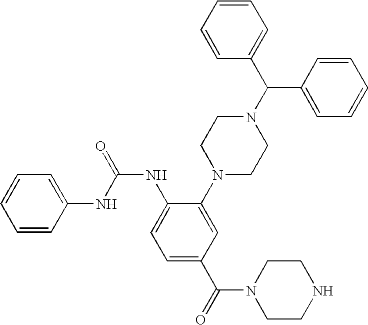

Cpd. No. 92

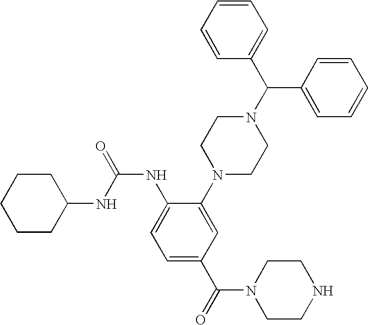

Cpd. No. 93

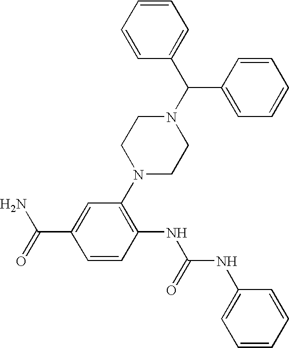

### Chemical Definitions & Nomenclature

As used herein, the following terms are intended to have the following meanings (additional definitions are provided throughout the Specification):

The term “C_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₋₃ denotes a radical containing 1, 2 or 3 carbon atoms.

The term “alkyl,” whether used alone or as part of a substituent group, refers to a saturated branched, or straight chain monovalent hydrocarbon radical derived by the removal of one hydrogen atom from a single carbon atom of a parent alkyl, alkene or alkyne. Typical alkyl groups include, but are not limited to, methyl, ethyl or propyl and the like and can be referred to as methanyl; ethanyl; propanyl (such as propan-1-yl, propan-2-yl, etc.) or butanyl (such as butan-1-yl, butan-2-yl, 2-methyl-propan-1-yl, 2-methyl-propan-2-yl, etc.) and the like. Where specific levels of unsaturation are intended, the nomenclature “alkenyl” and/or “alkynyl” is used, as defined below. In preferred embodiments, alkyl is (C₁₋₈)alkyl.

The term “alkenyl,” whether used alone or as part of a substituent group, refers to an unsaturated branched or straight chain monovalent hydrocarbon radical having at least one carbon-carbon double bond derived by the removal of one hydrogen atom from a single carbon atom of a parent alkene. The radical may be in either the cis or trans conformation about the double bond(s). Typical alkenyl groups include, but are not limited to, ethenyl, propenyl, butenyl and the like (such as prop-1-en-1-yl, prop-1-en-2-yl, prop-2-en-1-yl, prop-2-en-2-yl, but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl and the like). In preferred embodiments, alkenyl is (C₂₋₈)alkenyl.

The term “alkynyl,” whether used alone or as part of a substituent group, refers to an unsaturated branched, or straight chain monovalent hydrocarbon radical having at least one carbon-carbon triple bond derived by the removal of one hydrogen atom from a single carbon atom of a parent alkyne. Typical alkynyl groups include, but are not limited to, ethynyl, propynyl, butynyl and the like (such as prop-1-yn-1-yl, prop-2-yn-1-yl, but-1-yn-1-yl, but-1-yn-3-yl, but-3-yn-1-yl and the like). In preferred embodiments, the alkynyl group is (C₂₋₈)alkynyl.

The term “alkoxy” refers to a saturated or unsaturated, branched or straight chain monovalent hydrocarbon alcohol radical derived by the removal of the hydrogen atom from the hydroxide oxygen of an alcohol of a parent alkyl, alkene or alkyne. Where specific levels of saturation are intended, the nomenclature “alkoxy”, “alkenyloxy” and/or “alkynyloxy” is used consistent with the definitions of alkyl, alkenyl and alkynyl. In preferred embodiments, the alkoxy groups are (C₁₋₈)alkoxy groups.

The term “cycloalkyl” refers to saturated moncyclic hydrocarbon rings of from 3 to 20 carbon atom members (preferably, from 3 to 14 carbon atom members; more preferably, from 3 to 10 carbon atoms). Examples of cycloalkyl rings include, and are not limited to, cyclopropyl, cyclobutyl, cyclopentyl, cyclohexyl, cycloheptyl, adamantanyl, indanyl and the like. Where specific levels of saturation are intended, the terms “cycloalkyl” and “cycloalkenyl” are used consistent with the definition of alkyl and alkenyl.

The term “heterocyclyl” refers to a saturated monocyclic alkyl radical of from 5 to 9 ring members in which one or more ring carbon atoms are independently replaced with a heteroatom. Preferred heteroatoms to replace the carbon atom(s) are N, O or S. In preferred embodiments, 1, 2, 3 or 4 members of the ring are a nitrogen atom, or 0, 1, 2 or 3 members of the ring are nitrogen atoms and 1 member is an oxygen or sulfur atom. Examples of heterocyclyl rings include, and are not limited to, pyrrolidinyl, dioxolanyl, imidazolidinyl, pyrazolidinyl, tetrazolidinyl, piperidinyl, dioxanyl, morpholinyl, 1,4-dithianyl, thiomorpholinyl, piperazinyl, hexahydro-1,4-diazepinyl and the like.

The term “aryl” refers to a monovalent aromatic hydrocarbon radical derived by the removal of one hydrogen atom from a single carbon atom of a parent aromatic ring system. The term “parent aromatic ring system” refers to an unsaturated cyclic or polycyclic ring system having a conjugated π electron system. Specifically included within the definition of “parent aromatic ring system” are fused ring systems in which one or more rings are aromatic and one or more rings are saturated or unsaturated, such as, for example, naphthalene, indane, indene, phenalene, etc. Preferred aryl embodiments are derived from unsaturated or partially saturated monocyclic rings of 6 carbon members or from unsaturated or partially saturated fused ring systems of from 10 to 20 carbon members. Examples of aryl rings include, and are not limited to, phenyl, naphthalenyl, fluorenyl, indenyl, anthracenyl and the like.

The term “heteroaryl” refers to a monovalent heteroaromatic radical derived by the removal of one hydrogen atom from a single atom of a parent heteroaromatic ring system. The term “parent heteroaromatic ring system” refers to a parent aromatic ring system in which one or more carbon atoms are each independently replaced with a heteroatom. Preferred heteroatoms to replace the carbon atom(s) are N, P, O or S. Specifically included within the definition of “parent heteroaromatic ring systems” are fused ring systems in which one or more rings are heteroaromatic and one or more rings are saturated or unsaturated, such as, for example, indazole, indole, etc. Preferred heteroaryl embodiments include unsaturated or partially saturated monocyclic rings of from 5 to 9 ring members wherein the ring members consist of carbon atoms and at least one heteroatom. In other preferred embodiments, 1, 2, 3 or 4 members are nitrogen atoms or 0, 1, 2 or 3 members are nitrogen atoms and 1 member is an oxygen or sulfur atom. In other preferred embodiments, when allowed, up to two adjacent ring members are heteroatoms. Examples of heteroaryl rings include, and are not limited to, furyl, thienyl, pyrrolyl (including 2H-pyrrole, 2-pyrrolinyl or 3-pyrrolinyl), oxazolyl, thiazolyl, imidazolyl (including 2-imidazolinyl), pyrazolyl (including 2-pyrazolinyl), isoxazolyl, isothiazolyl, oxadiazolyl, triazolyl, thiadiazolyl, pyridinyl, pyridazinyl, pyrimidinyl, pyrazinyl and the like.

“Fused ring systems” include systems fused at adjacent ring atoms, those fused at a single ring atom and those fused at nonadjacent ring atoms. Preferrably, those fused on adjacent ring atoms form bicyclic or polycyclic ring systems, those fused on a single ring atom form spiro moieties and those fused on nonadjacent ring atoms form bridged ring systems. The types and amount of rings formed may be limited by available ring valences, starting materials or synthetic methods. However, all fused ring systems are intended to be included in the scope of the present compounds and associated synthetic methods.

Examples of fused cycloalkyl rings include adamantanyl, indanyl and the like. Examples of fused aryl rings include naphthalenyl, fluorenyl, indenyl, anthracenyl and the like. Examples of fused heterocyclyl rings include 1,3-benzodioxolyl, 2,3-dihydro-1,4-benzodioxinyl and the like. Examples of fused heteroaryl rings include indolyl, isoindolyl, indolinyl, benzofuryl, benzothienyl, indazolyl, benzimidazolyl, benzthiazolyl, benzoxazolyl, benzisoxazolyl, benzothiadiazolyl, benzotriazolyl, quinolizinyl, quinolinyl, isoquinolinyl, quinazolinyl and the like.

The term “point of attachment,” refers to a carbon atom within a radical which acts as the point of attachment for the radical to a core molecule; e.g., for a molecule C(O)—R, wherein a radical R is selected from a hydrogen or C₁₋₈alkyl, the C₁₋₈alkyl radical is attached to the molecule C(O)— by any carbon atom within the C₁₋₈alkyl chain. Accordingly, a variety of structures known to those with skill in the art are possible, such as C(O)CH₂CH₃ or C(O)CH(CH₃)₂.

The terms “secondary amine member” or “secondary amine atom” refer to a moiety of the formula Rₐ—NH—R_{b}, wherein the NH portion of the formula Rₐ—NH—R_{b} represents the secondary amine atom and, wherein Ra and Rb represent either identical or different adjacent atoms. The moiety is present in a heterocyclyl or heteroaryl ring system radical such as pyrrolyl, pyrrolinyl, pyrrolidinyl, imidazolyl, imidazolinyl, imidazolidinyl and the like. The secondary amine atom forms the point of attachment to a core molecule for the ring system radical in which it is present or the point of attachment for a substituent to the radical.

Where a radical is “substituted,” the term “substituted” refers to the independent replacement of one or more hydrogen atoms within the radical with that amount of substituents allowed by available valences. The term “independent(ly)” means that when a group or radical is substituted with more than one substituent that the substituents may be the same or different. Substitution is not limited to a terminal atom, but may occur within the radical or on a terminal atom.

The term “dependently substituted” means that the subsituents are specified in an indicated combination of structure variables.

Where a radical or group of radicals is refered to as being “optionally present,” the term “optionally present” refers to the replacement of one or more hydrogen atoms at a point of attachment on a core structure with that amount of radicals allowed by available valences; wherein, the point of attachment is otherwise saturated or aromatic when the radical(s) is (are) not present.

In general, IUPAC nomenclature rules are used throughout this disclosure. Nomenclature for radical substituents is derived by first indicating the functionality having the point of attachment with a hyphen, followed by the adjacent functionality toward the terminal portion of the side chain, as in:

—(C₁₋₆)alkyl-C(O)NH—(C₁₋₆)alkyl-Ph

or by describing the terminal portion of the side chain first, followed by the adjacent functionality toward the point of attachment, as in:

Ph—(C₁₋₆)alkylamido(C₁₋₆)alkyl

either of which refers to a radical of the formula:

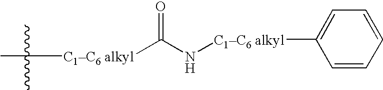

Compounds exemplified in the present invention were named according to nomenclature well known in the art, either using Autonom (brand of nomenclature software provided in the ChemDraw Ultra® office suite marketed by CambridgeSoft.com) or using ACD/Index Name™ (brand of commercial nomenclature software marketed by Advanced Chemistry Development, Inc., Toronto, Ontario).

### Pharmaceutical Preparations & Methods of Use

The compounds of the present invention may also be present in the form of pharmaceutically acceptable salts. For use in medicine, the salts of the compounds of this invention refer to non-toxic “pharmaceutically acceptable salts.” FDA approved pharmaceutically acceptable salt forms (Ref. International J. Pharm. 1986, 33, 201-217; J. Pharm. Sci., 1977, January, 66(1), p1) include pharmaceutically acceptable acidic/anionic or basic/cationic salts.

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH3, NH₄OH, N-methyl-D-glucamine, piperidine, potassium, potassium-t-butoxide, potassium hydroxide (aqueous), procaine, quinine, SEH, sodium, sodium carbonate, sodium-2-ethylhexanoate, sodium hydroxide, triethanolamine (TEA) or zinc.

The present invention includes within its scope prodrugs of the compounds of this invention. In general, such prodrugs will be functional derivatives of the compounds, which are readily convertible in vivo into an active compound. Thus, in the methods of treatment of the present invention, the term “administering” shall encompass the treatment of the various disorders described with the compound specifically disclosed or a compound, or prodrug thereof, which would be obviously included within the scope of the invention although not specifically disclosed for certain of the instant compounds. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such stereoisomers and mixtures thereof are encompassed within the scope of the present invention. The terms “S” and “R,” when used herein for indicating stereoisomer configuration, are as defined in the literature (IUPAC Recommendations for Fundamental Stereochemistry (Section E), Pure Appl. Chem., 1976, 45:13-30).

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

Embodiments of the present invention comprise the use of compounds that are phospholipase inhibitors for treating or ameliorating an inflammatory disorder. The term phospholipase refers to any one of the subtypes of the class of phospholipases activated following binding of a ligand to its cell surface receptor, such as phospholipase C, phospholipase C-β1 or phospholipase C-β2.

An embodiment of the present invention comprises the use of compounds that are selective phospholipase inhibitors for treating or ameliorating an inflammatory disorder. The usefulness of a compound of formula (I) as a phospholipase inhibitor can be determined according to the methods disclosed herein and the scope of such usefulness includes use in a plurality of inflammatory disorders.

An embodiment of the present invention comprises the use of compounds that are selective phospholipase C inhibitors for treating or ameliorating an inflammatory disorder. Another embodiment of the present invention comprises the use of compounds that are selective phospholipase C-β inhibitors useful for treating or ameliorating an inflammatory disorder.

Embodiments of the present invention include a method for treating or ameliorating an inflammatory disorder in a subject in need thereof comprising administering to the subject a therapeutically effective amount of a compound of formula (I) or composition thereof. An embodiment further includes a method for treating or ameliorating an inflammatory disorder in a subject in need thereof comprising administering to the subject a prophylactically effective amount of a compound of formula (I) or composition thereof.

The term “subject” as used herein, refers to an animal, preferably a mammal, most preferably a human, which has been the object of treatment, observation or experiment and is at risk of (or susceptible to) developing an inflammatory disorder or having an inflammatory disorder.

The term “administering” is to be interpreted in accordance with the methods of the present invention. Such methods include therapeutically or prophylactically administering an effective amount of a composition or medicament of the present invention at different times during the course of a therapy or concurrently in a combination form. Prophylactic administration can occur prior to the manifestation of symptoms characteristic of an inflammatory disorder such that the disorder is prevented or, alternatively, delayed in its progression. The methods of the present invention are further to be understood as embracing all therapeutic or prophylatic treatment regimens used by those skilled in the art.

The terms “therapeutically effective amount” or “prophylactically effective amount” refer to that amount of active compound or pharmaceutical agent that elicits the biological or medicinal response in a tissue system, animal or human, that is being sought by a researcher, veterinarian, medical doctor, or other clinician, which includes alleviation of the symptoms of the disease or disorder being treated.

The term “inflammatory disorder” refers to disorders and diseases associated with an inflammatory response such that there is discomfort or decreased life expectancy to the organism. Such disorders and diseases occur in humans, and in various species of animals, and include, but are not limited to, autoimmune diseases (including but not limited to rheumatoid arthritis, systemic lupus erythematosus, inflammatory bowel diseases such as Crohn's disease and ulcerative colitis, multiple sclerosis, asthma, Graves' disease, myasthenia gravis, and ankylosing spondylitis); rejection of tissue or organ allografts (including but not limited to kidney, heart, liver, lung, whole pancreas, pancreatic islets, and corneas); infectious diseases (including but not limited to HIV-related diseases [eg AIDS] and tuberculosis); allergic diseases (including but not limited to hay fever, latex allergies, food allergies, and pet allergies); various inflammatory skin conditions (including but not limited to psoriasis, dernatis, eczema, poison ivy), neoplastic diseases (eg cancer), and vascular disorders (including but not limited to atherosclerosis and restenosis).

Another embodiment for use of the compounds of the present invention is a method for treating or ameliorating restenosis wherein a phospholipase inhibitor is impregnated on the surface of a medical device such as an angioplasty balloon or stent, thus targeting drug delivery to the local environment. Coronary angioplasty or stent implantation are otherwise highly effective procedures which reduce the severity of vascular abnormalities, but long-term success is limited by a high rate of restenosis. Accordingly, an example of a preferred use includes use of a phospholipase inhibitor on an angioplasty balloon or on a stent where restenotic endothelial and smooth muscle cell proliferation are the leading cause of vascular reocclusion.

An embodiment of the invention includes a composition or medicament comprising a mixture one or more compounds of the present invention and an optional pharmaceutically acceptable carrier.

The term “composition” refers to a product containing a compound of the present invention (such as a product comprising the specified ingredients in the specified amounts, as well as any product which results, directly or indirectly, from such combinations of the specified ingredients in the specified amounts). The term “medicament” refers to a product for use in treating or ameliorating an inflammatory disorder or condition mediated by PLC-β2.

The term “pharmaceutically acceptable” refers to molecular entities and compositions that are of sufficient purity and quality for use in the formulation of a composition or medicament of the present invention. Since both human use (clinical and over-the-counter) and veterinary use are equally included within the scope of the present invention, a formulation would include a composition or medicament for either human or veterinary use.

Embodiments include a process for making the composition or medicament comprising mixing any of the instant compounds and a pharmaceutically acceptable carrier and include those compositions or medicaments resulting from such a process. Contemplated processes include both conventional and unconventional pharmaceutical techniques. Other embodiments include a composition or medicament comprising a mixture of at least two of the instant compounds in association with a pharmaceutically acceptable carrier.

The composition or medicament may be administered in a wide variety of dosage unit forms depending on the method of administration; wherein such methods include (without limitation) oral, sublingual, nasal (inhaled or insufflated), transdermal, rectal, vaginal, topical (with or without occlusion), intravenous (bolus or infusion) or for injection (intraperitoneally, subcutaneously, intramuscularly, intratumorally or parenterally) using a suitable dosage form well known to those of ordinary skill in the area of pharmaceutical administration. Accordingly, the term dosage unit or dosage form is used to refer to (without limitation) a tablet, pill, capsule, solution, syrup, elixir, emulsion, suspension, suppository, powder, granule or sterile solution, emulsion or suspension (for injection [from an ampule or using a device such as an auto-injector] or for use as an aerosol, spray or drop). Furthermore, the composition may be presented in a form suitable for weekly or monthly administration: e.g. an insoluble salt of the active compound (such as the decanoate salt) may be adapted to provide a depot preparation for intramuscular injection.

In preparing a dosage form, the principal active ingredient (such as a compound of the present invention or a pharmaceutically acceptable salt thereof) is optionally mixed with one or more pharmaceutical carriers (such as a starch, sugar, diluent, granulating agent, lubricant, glidant, binder, disintegrating agent and the like), one or more inert pharmaceutical excipients (such as water, glycols, oils, alcohols, flavoring agents, preservatives, coloring agents, syrup and the like), one or more conventional tableting ingredient (such as corn starch, lactose, sucrose, sorbitol, talc, stearic acid, magnesium stearate, dicalcium phosphate, any of a variety of gums and the like) and a diluent (such as water and the like) to form a homogeneous composition (whereby the active ingredient is dispersed evenly throughout the mixture) which may be readily subdivided into dosage units containing equal amounts of a compound of the present invention.

Binders include, without limitation, starch, gelatin, natural sugars (such as glucose, beta-lactose and the like), corn sweeteners and natural and synthetic gums (such as acacia, tragacanth, sodium oleate, sodium stearate, magnesium stearate, sodium benzoate, sodium acetate, sodium chloride and the like). Disintegrating agents include, without limitation, starch, methyl cellulose, agar, bentonite, xanthan gum and the like.

Because of their ease of administration, tablets and capsules represent an advantageous oral dosage unit form, wherein solid pharmaceutical carriers are employed. If desired, tablets may be sugarcoated or enteric-coated by standard techniques. Tablets may also be coated or otherwise compounded to provide a prolonged therapeutic effect. For example, the dosage form may comprise an inner dosage and an outer dosage component, whereby the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and nonenteric layer or coating materials may be used (such as polymeric acids, shellacs, acetyl alcohol, cellulose acetate and the like) or combinations thereof.

The compound of formula (I) may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin. The liquid forms in suitably flavored suspending or dispersing agents may also include the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

As is also known in the art, the compounds may alternatively be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution or an isotonic aqueous saline solution. Other optional ingredients include vegetable oils (such as peanut oil, cottonseed oil, sesame oil and the like) and organic solvents (such as solketal, glycerol, formyl and the like). Alternatively, a sterile non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers or pain-soothing agents. Injectable suspensions may also be prepared, in which case appropriate liquid carriers, suspending agents and the like may be employed.

Compounds of the present invention may be administered intranasally using a suitable intranasal vehicle. Compounds of the present invention may be administered topically using a suitable topical transdermal vehicle or a transdermal patch. Administration via a transdermal delivery system requires a continuous rather than intermittent dosage regimen.

Compounds of the present invention may also be administered via a slow release composition; wherein, the composition includes a biodegradable slow release carrier (typically, a polymeric carrier) and a compound of the invention. Slow release carriers are well known in the art and are used to form particles that capture therein an active compound(s) and slowly degrade/dissolve in a suitable environment (e.g., aqueous, acidic, basic, etc). Such particles are useful because they degrade/dissolve in body fluids and release the active compound(s) therein. The particles are preferably nanoparticles (i.e., in the range of about 1 to 500 nm in diameter, preferably about 50-200 nm in diameter, and most preferably about 100 nm in diameter). In a process for preaparing a slow release composition, a slow release carrier and a compound of the invention are first dissolved or dispersed in an organic solvent. The resulting mixture is added into an aqueous solution containing an optional surface-active agent(s)to produce an emulsion. The organic solvent is then evaporated from the emulsion to provide a colloidal suspension of particles containing the slow release carrier and the compound of the invention.

As previously described, a contemplated embodiment of the dosage unit will contain an amount of an active ingredient or prodrug thereof necessary to be therapeutically effective for symptomatic relief to a subject in need thereof. A therapeutically effective amount of the active compound in the dosage unit may range from about 0.001 mg to about 1000 mg and may be constituted into any form suitable for the administration method and regimen selected for the subject. Depending on the subject and disease to be treated, the therapeutically effective amount may range from about 0.0001 mg/kg to 300 mg/kg of body weight per day; or, from about 0.0005 to about 100 mg/kg of body weight per day; or, from about 0.001 to about 50 mg/kg of body weight per day. An optimal therapeutically effective amount and administration method and regimen may be readily determined by those skilled in the art, and will vary depending on factors associated with the particular patient being treated (age, weight, diet and time of administration), the severity of the condition being treated, the compound and dosage unit being employed, the mode of administration and the strength of the preparation. Dosage unit(s) may be administered to achieve the therapeutically effective amount in a regimen of from about once per day to about 5 times per day. The preferred dosage unit for oral administration is a tablet containing, 0.01, 0.05, 0.1, 0.5, 1.0, 2.5, 5.0, 10.0, 15.0, 25.0, 50.0, 100, 150, 200, 250 or 500 mg of the active ingredient.

### Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art. No attempt has been made to optimize the yields obtained in any of the example reactions. One skilled in the art would know how to increase such yields through routine variations in reaction times, temperatures, solvents and/or reagents.

The terms used in describing the invention are commonly used and known to those skilled in the art. When used herein, the following abbreviations have the indicated meanings:

Ac-BSA or BSA acylated bovine serum albumin or bovine serum albumin

Bn benzyl

Cpd compound

DIBAL diisobutylaluminum hydride

DIC 1,3-diisopropyl carbodiimide

DEAD diethylazodicarboxylate

DMF N,N-dimethyl formamide

DMSO dimethyl sulfoxide

DPPF 1,1′-bis(diphenylphosphini)ferrocene

EDIC 1-(3-dimethylaminopropyl)-3-ethylcarbodiimide

Et ethyl

HOBt 1-hydroxybenzotriazole

LDA lithium diisopropylamide

Me methyl

min/h/rt/mp minute/hour/room temperature/melting point

Ph or PH phenyl

Pd₂(dba)₃ tris(dibenzylideneacetone)dipalladium(0)

Py pyridine

TFA trifluoroacetic acid

THF tetrahydrofuran

TMEDA tetramethylethylenediamine

TPP triphenylphosphine

All commercially available chemicals were obtained from commercial suppliers and used without further purification. Particular components, such as the peptide reaction vessels (obtained from NovaBiochem), the Wang resin (also from Novabiochem, 70-90 mesh), Rink resin and the wrist action shaker (obtained from Burrell Scientific Co.) used in the examples are also commercially available.

### Scheme A

Solid Phase Synthesis of Amido and Piperazinyl Substituted Anilino Compounds

In accordance with Scheme A, a commercially available Rink resin was reacted with piperidine to provide a resin-bound amide. Depending on the target compound desired, a commercially available Wang resin may also be used (See Scheme B). Other starting materials may also be used for both solid and solution based synthesis, thus providing a variety of equivalent substituent substitutions which are intended to be included within the scope of the present invention.

The amidated resin was then coupled with a nitro substituted benzoic acid Compound A1 to yield a resin-bound Compound A2. The Compound A2 fluoro atom was replaced with a substituted Compound A3 (where n is preferably 1) to produce a piperazinyl substituted Compound A4. The Compound A4 nitro group was reduced to give the corresponding piperazinyl substituted anilino Compound A5. A reactive compound such as an R₄—N═C═R₃ moiety (where R₃ and R₄ are as defined herein) was reacted with Compound A5 to provide a Compound A6.

Cleavage of Compound A6 from the solid support resin yielded a deprotected amido Compound A7. The amido nitrogen atom may be further substituted by reacting Compound A7 with a compound such as R₁—CA, wherein CA is a Coupling Agent to provide a target Compound A8 representative of formula (Ia).

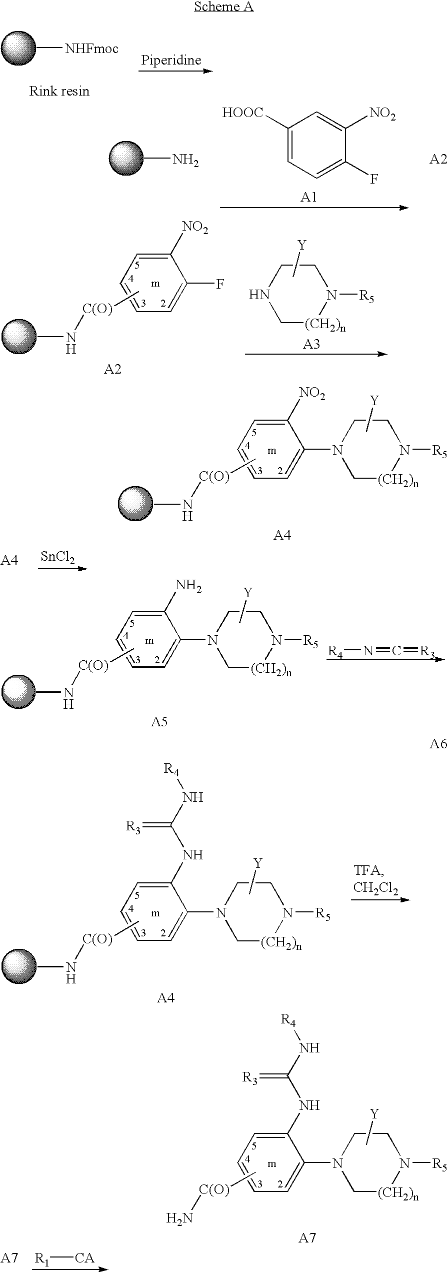

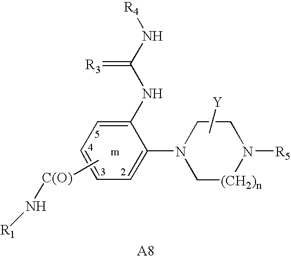

### EXAMPLE 1

### 4-[4-(diphenylmethyl)-1-piperazinyl]-3-[[(phenylamino)carbonyl]amino]-benzamide (Cpd 8)

Commercially available Fmoc protected Rink resin (0.5 g, 0.3 mnmol) and a 40% piperidine:dimethylformamide (DMF) (v/v) solution (5 mL, 0.6 mmol/g) were added to a peptide reaction vessel. The mixture was shaken for 1 h using a wrist action shaker and the DMF was removed by vacuum filtration. The 40% piperidine:DMF solution (5 mL) was again added to the mixture and shaken for 30 min. The DMF was removed by vacuum filtration and the reaction product was sequentially washed with an excess of DMF, CH₂Cl₂ and MeOH, then a final wash with CH₂Cl₂ to provide a resin bound amine Compound 1a used in the next step without characterization.

A 4-fluoro-3-nitrobenzoic acid Compound 1b (2.31 g, 12.5 mmol) and 1-hydroxybenzotriazole (1.69 g, 12.5 mmol) were added in one portion to a 50 mL round bottom flask containing DMF (10 mL) and CH₂Cl₂ (10 mL) followed by 1,3-diisopropylcarbodiimide (1.95 mL, 12.5 mmol). The solution was stirred for 30 min and added to the 50 mL reaction vessel containing Compound 1a (2.5 g, 1.25 mmol). The mixture was shaken for 16 h and the solvent was removed by vacuum filtration. The reaction product was sequentially washed with an excess of DMF, CH₂Cl₂ and MeOH, then a final wash with CH₂Cl₂ to give a resin-bound benzamide Compound 1c. To characterize Compound 1c, an aliquot of the washed product (20 mg) was cleaved from the resin using 50% TFA in CH₂Cl₂ (1 mL), shaken for 1 h and filtered, then washed with MeOH and characterized: ESMS m/z 185 (M⁺H).

DMF (2 mL) and a 1-benzhydrylpiperazine Compound 1d (0.252 g, 1 mmol) were added to the reaction vessel containing Compound 1c (0.2 g, ˜0.1 mmol), then diisopropylethylamine (0.174 mL, 1 mmol) was added. The mixture was shaken over a 2 day period and turned from a pale yellow color to a yellow-orange color, then the solvent was removed by vacuum filtration. The reaction product was sequentially washed with an excess of DMF, CH₂Cl₂ and MeOH, then a final wash with CH₂Cl₂ to give a resin-bound benzamido substituted piperazine Compound 1e. To characterize Compound 1e, an aliquot of the washed product (20 mg) was cleaved from the resin using 50% TFA in CH₂Cl₂ (1 mL), shaken for 1 h and filtered, then washed with MeOH and characterized: ESMS m/z 417 (M⁺H).

DMF (2 mL) and tin(II) chloride dihydrate (0.45 g, 2 mmol) were added to the reaction vessel containing Compound 1e (0.2 g, ˜0.1 mmol). The mixture was shaken overnight and turned from a yellow-orange color to almost colorless, then the solvent was removed by vacuum filtration. The reaction product was sequentially washed with an excess of DMF, CH₂Cl₂ and MeOH, then a final wash with CH₂Cl₂ to give a resin-bound aminated Compound if. To characterize Compound if, an aliquot of the washed product (20 mg) was cleaved from the resin using 50% TFA in CH₂Cl₂ (1 mL), shaken for 1 h and filtered, then washed with MeOH and characterized: ESMS m/z 387 (M⁺H). Phenyl isocyanate (17.85 mg, 0.15 mmol) was added to the reaction vessel containing Compound 1f (0.06 g, ˜0.03 mmol) and CH₂Cl₂ (2 mL). The mixture was shaken for 48 h and the solvent was removed by vacuum filtration. The reaction product was sequentially washed with an excess of DMF, CH₂Cl₂ and MeOH, then a final wash with CH₂Cl₂ to give a resin-bound amino substituted Compound 1g. The washed Compound 1g was cleaved from the resin using 50% TFA in CH₂Cl₂ (1 mL), shaken for 1 h and filtered, then washed with MeOH. The filtrates were combined and concentrated to provide a crude trifluoroacetate salt. The salt was purified by column chromatography on silica gel (9:1 CH₂Cl₂:MeOH was used as the eluent) to provide Compound 8 (10.5 mg, 69% yield) as a pale yellow solid. ESMS m/z 506 (M⁺H).

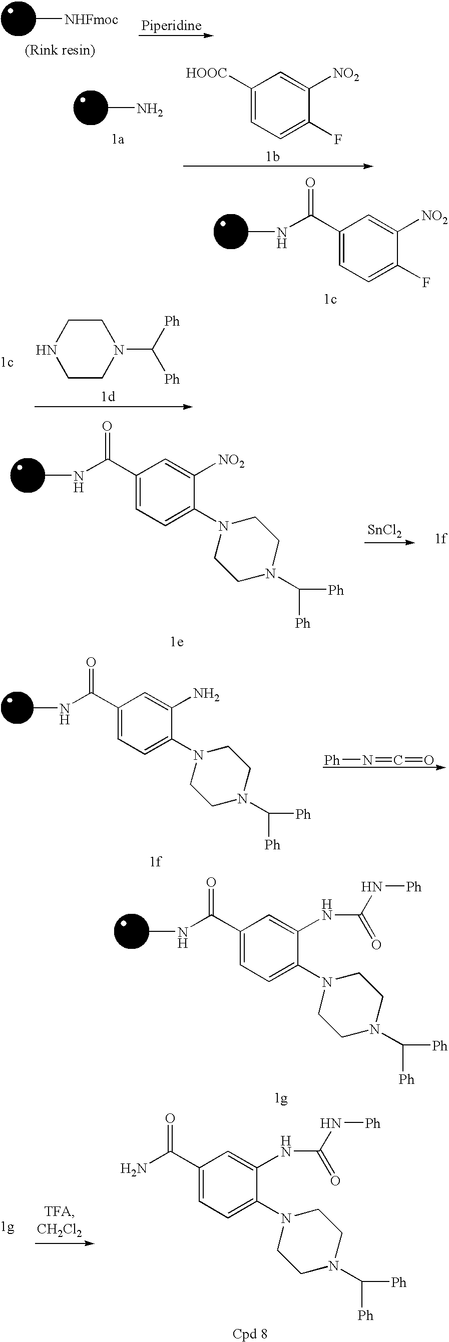

Using the procedure of Example 1 and the appropriate reagents and starting materials known to those skilled in the art, other compounds of the present invention may be prepared including, but not limited to (MS: Mass Spec data as MS m/z MH⁺):

| Cpd | Name | MS |
|---|---|---|
| 1 | 4-[4-(2-methoxyphenyl)-1-piperazinyl]-3- | 446 |
| | [[(phenylamino)carbonyl]amino]-benzamide | |
| 2 | 3-[[(phenylamino)carbonyl]amino]-4-[4-(phenylmethyl)-1- | 430 |
| | piperazinyl]-benzamide | |
| 3 | 4-[4-[bis(4-fluorophenyl)methyl]-1-piperazinyl]-3- | 542 |
| | [[(phenylamino)carbonyl]amino]-benzamide | |
| 4 | 4-[4-[bis(4-fluorophenyl)methyl]-1-piperazinyl]-3-[[[(2- | 560 |
| | fluorophenyl)amino]carbonyl]amino]-benzamide | |
| 5 | 4-[4-(diphenylmethyl)-1-piperazinyl]-3-[[[(4- | 551 |
| | nitrophenyl)amino]carbonyl]amino]-benzamide | |
| 6 | 4-[4-(diphenylmethyl)-1-piperazinyl]-3- | 520 |
| | [[[(phenylmethyl)amino]carbonyl]amino]-benzamide | |
| 7 | 3-[[[(3,5-dimethylphenyl)amino]carbonyl]amino]-4-[4- | 534 |
| | (diphenylmethyl)-1-piperazinyl]-benzamide | |
| 9 | 4-[4-(9H-fluoren-9-yl)-1-piperazinyl]-3- | 504 |
| | [[(phenylamino)carbonyl]amino]-benzamide | |
| 10 | 3-[[(cyclohexylamino)carbonyl]amino]-4-[4- | 512 |
| | (diphenylmethyl)-1-piperazinyl]-benzamide | |
| 11 | 4-[4-(diphenylmethyl)-1-piperazinyl]-3-[[[[(1S)-1- | 534 |
| | phenylethyl]amino]carbonyl]amino]-benzamide | |
| 12 | 3-[[(butylamino)carbonyl]amino]-4-[4-(diphenylmethyl)-1- | 486 |
| | piperazinyl]-benzamide | |
| 13 | 4-[4-(diphenylmethyl)-1-piperazinyl]-3-[[[(4- | 524 |
| | fluorophenyl)amino]carbonyl]amino]-benzamide | |
| 14 | 3-[[(1,3-benzodioxol-5-ylamino)carbonyl]amino]-4-[4- | 586 |
| | [bis(4-fluorophenyl)methyl]-1-piperazinyl]-benzamide | |
| 15 | 4-[4-[bis(4-fluorophenyl)methyl]-1-piperazinyl]-3-[[[(2,4- | 570 |
| | dimethylphenyl)amino]carbonyl]amino]-benzamide | |
| 16 | 4-[4-[bis(4-fluorophenyl)methyl]-1-piperazinyl]-3-[[[(1- | 570 |
| | phenylethyl)amino]carbonyl]amino]-benzamide | |
| 17 | 4-[4-[bis(4-fluorophenyl)methyl]-1-piperazinyl]-3-[[[(2- | 572 |
| | methoxyphenyl)amino]carbonyl]amino]-benzamide | |
| 18 | 4-[4-[bis(4-fluorophenyl)methyl]-1-piperazinyl]-3-[[[(2,4- | 602 |
| | dimethoxyphenyl)amino]carbonyl]amino]-benzamide | |
| 19 | 4-[4-[bis(4-fluorophenyl)methyl]-1-piperazinyl]-3-[[[[4- | 585 |
| | (dimethylamino)phenyl]amino]carbonyl]amino]-benzamide | |
| 20 | 4-[4-[bis(4-fluorophenyl)methyl]-1-piperazinyl]-3-[[[(4- | 572 |
| | methoxyphenyl)amino]carbonyl]amino]-benzamide | |
| 21 | 4-[4-[bis(4-fluorophenyl)methyl]-1-piperazinyl]-3- | 572 |
| | [[[(phenylmethyl)amino]thioxomethyl]amino]-benzamide | |
| 22 | 4-[4-[bis(4-fluorophenyl)methyl]-1-piperazinyl]-3- | 558 |
| | [[(phenylamino)thioxomethyl]amino]-benzamide | |
| 77 | 4-[4-[(4-fluorophenyl)-4-pyridinylmethyl]-1-piperazinyl]-3- | 525 |
| | [[(phenylamino)carbonyl]amino]-benzamide | |
| 78 | 3-[[cyclohexylamino)carbonyl]amino]-4-[4-[(4- | 531 |
| | fluorophenyl)-4-pyridinylmethyl]-1-piperazinyl]-benzamide | |
| 79 | 4-[4-[(4-fluorophenyl)-4-pyridinylmethyl]hexahydro-1H- | 539 |
| | 1,4-diazepin-1-yl]-3-[[(phenylamino)carbonyl]amino]- | |
| | benzamide | |
| 80 | 3-[[(cyclohexylamino)carbonyl]amino]-4-[4-[(4- | 545 |
| | fluorophenyl)-4-pyridinylmethyl]hexahydro-1H-1,4- | |
| | diazepin-1-yl]-benzamide | |
| 81 | 4-[4-[bis(4-fluorophenyl)methyl]hexahydro-1H-1,4- | 556 |
| | diazepin-1-yl]-3-[[(phenylamino)carbonyl]amino]- | |
| | benzamide | |
| 82 | 4-[4-[bis(4-fluorophenyl)methyl]hexahydro-1H-1,4- | 562 |
| | diazepin-1-yl]-3-[[(cyclohexylamino)carbonyl]amino]- | |
| | benzamide | |

### Scheme B

Solid Phase Synthesis of Piperazinyl and Piperazinoyl Substituted Anilino Compounds

In accordance with Scheme B, a commercially available Wang resin Compound B1 was reacted with piperazine to provide a resin-bound Compound B2. Other starting materials may also be used for both solid and solution based synthesis, thus providing a variety of equivalent substituent substitutions which are intended to be included within the scope of the present invention.

Compound B2 was coupled with the nitro substituted benzoic acid Compound Al to yield a resin-bound Compound B3. The Compound B3 fluoro atom was replaced with a substituted Compound A3 (where n is preferably 1) to produce a piperazinyl-piperazinoyl substituted Compound B4. The Compound B4 nitro group was reduced to give the corresponding piperazinyl-piperazinoyl substituted anilino Compound B5. A compound such as R₄—N═C═R₃ Compound B6 (where R₃ and R₄ are as defined herein) was reacted with Compound B5 to provide a Compound B7.

Cleavage of Compound B7 from the solid support resin yielded a Compound B8. The deprotected piperazinoyl nitrogen atom was further substituted by reacting Compound B8 with an R₂ substituted coupling agent Compound B9 to provide a target Compound B10 presentative of formula (Ib).

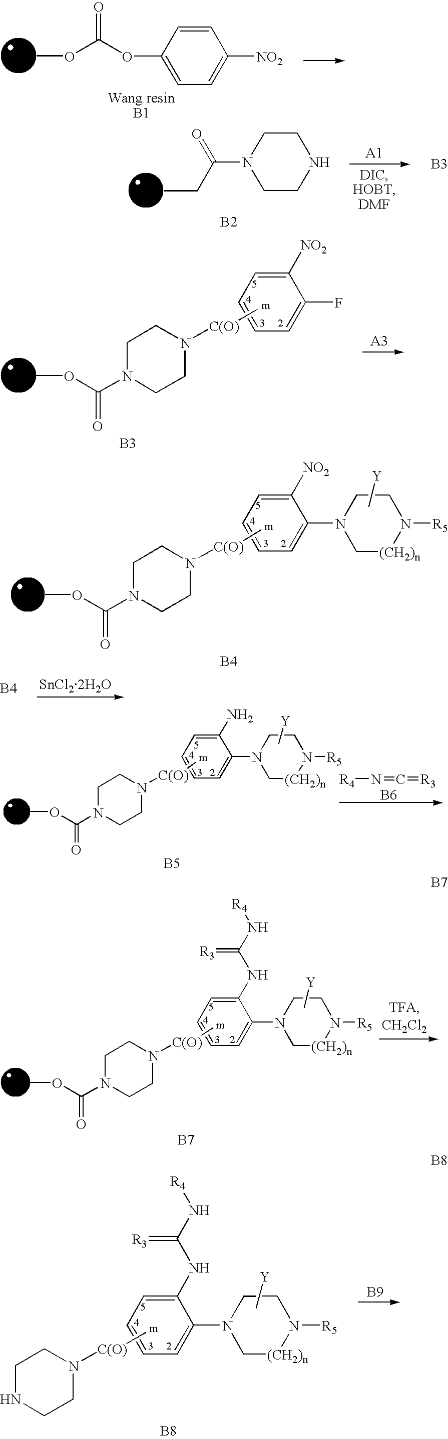

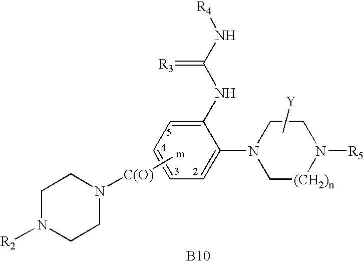

### EXAMPLE 2

### N-(2-aminoethyl)-4-[4-(diphenylmethyl)-1-piperazinyl]-3-[[(phenylamino)carbonyl]amino]-benzamide (Cpd 26)

### N-[2-(dimethylamino)ethyl]-4-[4-(diphenylmethyl)-1-piperazinyl]-3-[[(phenylamino)carbonyl]amino]-benzamide (Cpd 29)

Commercially available p-nitrophenyl carbonate Wang resin (1.0 g, 1.1 mmol, 0.92 mmol/g) in DMF (10 mL) and ethylene diamine (1.0 g, 17.8 mmol) were added to a reaction vessel. The mixture was shaken for 16 h using a wrist action shaker and the DMF was removed by vacuum filtration. The reaction product was sequentially washed with an excess of DMF, MeOH and CH₂Cl₂ until the filtrate did not exhibit a yellow color to provide a resin-bound amine Compound 2a used in the next step without characterization.

A 4-fluoro-3-nitrobenzoic acid Compound 1b (0.96 g, 9.2 mmol) and 1-hydroxybenzotriazole (HOBT) (1.3 g, 9.0 mmol) were added in one portion to a 100 mL round bottom flask containing DMF (10 mL) and CH₂Cl₂ (10 mL). The solution was stirred under argon for 5 min and 1.4 mL (9.0 mmol) of 1,3-diisopropylcarbodiimide (DIC) was added dropwise. The mixture was then stirred for 30 min and added to the reaction vessel containing Compound 2a. The mixture was shaken for 16 h and the solvent was removed by vacuum filtration. The reaction product was sequentially washed with an excess of DMF, CH₂Cl₂ and MeOH, then a final wash with CH₂Cl₂ to obtain a substituted benzamido Compound 2b.

DMF (5 mL) and the benzhydrylpiperazine Compound 1d (1.5 g, 5.96 mmol) were added to the reaction vessel containing Compound 2b (approximately 0.46 mmol), then diisopropylethylamine (1.0 mL, 6.4 mmol) was added. The mixture was shaken overnight and turned from a pale yellow color to a yellow-orange color, then the solvent was removed by vacuum filtration. The reaction product was sequentially washed with an excess of DMF, CH₂Cl₂ and MeOH, then a final wash with CH₂Cl₂ to give a nitro substituted benzamido piperazinylene Compound 2c.

DMF (10 mL) and tin(II) chloride dihydrate (1.2 g, 5.3 mmol) were added in one portion to the reaction vessel containing Compound 2c (0.5 g, 0.46 mmol). The mixture was shaken overnight and turned from a yellow-orange color to almost colorless, then the solvent was removed by vacuum filtration. The reaction product was sequentially washed with an excess of DMF, CH₂Cl₂ and MeOH, then a final wash with CH₂Cl₂ to give a resin-bound aminated Compound 2d.

Phenyl isocyanate (1.0 mL, 7.0 mmol) was added to the reaction vessel containing Compound 2d (0.23 mmol) and CH₂Cl₂ (10 mL). The mixture was shaken overnight and the solvent was removed by vacuum filtration. The reaction product was sequentially washed with an excess of DMF, CH₂Cl₂ and MeOH, then a final wash with CH₂Cl₂ to give a resin-bound phenyl urea Compound 2e. The washed Compound 2e was cleaved from the resin using 5% TFA (20 mL) in CH₂Cl₂ (1 mL), shaken for 30 min and filtered, then washed with CH₂Cl₂ and MeOH. The filtrates were combined and concentrated to provide Compound 26 as a trifluoroacetate salt. ESMS m/e 549 (M+1, 90% ), 383 (M−PhCHPh, 100% ), 167 (PhCHPh, 20% ).

Compound 26 (0.04 mmol, 0.03 g) was then dissolved in CH₂Cl₂ (2 mL) and MeOH (2 drops) and treated with a 37% CH₂(O) (formaldehyde) solution (in water) followed by NaB(OAc)₃H (0.4 mmol, 0.08 g) to form a suspension. The suspension was stirred at rt for 1 h, then diluted with a 10% NaOH solution and extracted with CH₂Cl₂. The organic extracts were dried over Na₂SO₄ and the solvent was removed to obtain Compound 29 as a free base. ESMS m/e 577 (M+1, 100% ). The residue was dissolved in CH₂Cl₂ and then treated with HCl (3 Eq.) in Et₂O. The solvent was removed in vacuo to obtain Compound 29 as a hydrochloride salt.

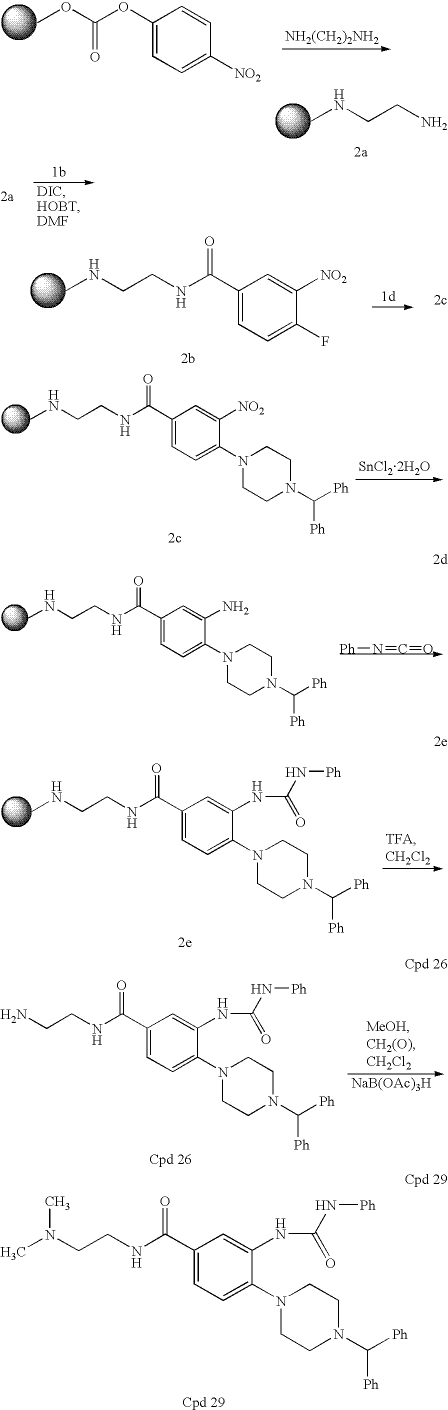

Using the procedures of Example 2 and the appropriate reagents and starting materials known to those skilled in the art, other compounds of the present invention were prepared including, but not limited to (MS: Mass Spec data as MS m/z MH⁺):

| Cpd | Name | MS |
|---|---|---|
| 23 | N-[2-[4-(diphenylmethyl)-1-piperazinyl]-5-[(4-methyl-1- | 589 |
| | piperazinyl)carbonyl]phenyl]-N′-phenylurea | |
| 24 | N-[2-[4-(diphenylmethyl)-1-piperazinyl]-5-[(hexahydro-1H-1,4- | 589 |
| | diazepin-1-yl)carbonyl]phenyl]-N′-phenylurea | |
| 25 | N-cyclohexyl-N′-[2-[4-(diphenylmethyl)-1-piperazinyl]-5- | 595 |
| | [(hexahydro-1H-1,4-diazepin-1-yl)carbonyl]-phenyl]urea | |
| 27 | N-(2-aminoethyl)-3-[[(cyclohexylamino)carbonyl]amino]-4-[4-(diphenylmethyl)- | 555 |
| | 1-piperazinyl]-benzamide | |
| 28 | N-cyclohexyl-N′-[2-[4-(diphenylmethyl)-1-piperazinyl]-5-[(4-methyl-1- | 595 |
| | piperazinyl)carbonyl]-phenyl]urea | |
| 30 | 3-[[(cyclohexylamino)carbonyl]amino]-N-[2- | 583 |
| | (dimethylamino)ethyl]-4-[4-(diphenylmethyl)-1-piperazinyl]-benzamide | |
| 31 | N-cyclohexyl-N′-[2-[4-(diphenylmethyl)-1-piperazinyl]-5- | 609 |
| | [(hexahydro-4-methyl-1H-1,4-diazepin-1-yl)carbonyl]phenyl]-urea | |
| 38 | N-cyclohexyl-N′-[2-[4-(diphenylmethyl)-1-piperazinyl]-5-(1- | 581 |
| | piperazinylcarbonyl)phenyl]-urea | |
| 39 | N-[2-[4-(diphenylmethyl)-1-piperazinyl]-5-(1- | 589 |
| | piperazinylcarbonyl)phenyl]-N′-(phenylmethyl)urea | |
| 40 | N-[2-[4-(diphenylmethyl)-1-piperazinyl]-5-(1- | 575 |
| | piperazinylcarbonyl)phenyl]-N′-phenylurea | |
| 41 | N-(2,4-dimethylphenyl)-N′-[2-[4-(diphenylmethyl)-1-piperazinyl]-5-(1- | 603 |
| | piperazinylcarbonyl)phenyl]-urea | |
| 42 | N-(3,5-dimethylphenyl)-N′-[2-[4-(diphenylmethyl)-1-piperazinyl]-5-(1- | 603 |
| | piperazinylcarbonyl)phenyl]-urea | |
| 43 | N-[2-[4-(diphenylmethyl)-1-piperazinyl]-5-(1-piperazinylcarbonyl)phenyl]- | 605 |
| | N′-(4-methoxyphenyl)urea | |
| 44 | N-[2-[4-(9H-fluoren-9-yl)-1-piperazinyl]-5-(1- | 573 |
| | piperazinylcarbonyl)phenyl]-N′-phenylurea | |
| 45 | N-[2-[4-[(4-chlorophenyl)phenylmethyl]-1-piperazinyl]-5-(1- | 616 |
| | piperazinylcarbonyl)phenyl]-N′-cyclohexyl-urea | |
| 46 | N-[2-[4-[bis(4-fluorophenyl)methyl]-1-piperazinyl]-5-(1- | 611 |
| | piperazinylcarbonyl)phenyl]-N′-phenylurea | |
| 47 | N-[2-[4-[bis(4-fluorophenyl)methyl]-1-piperazinyl]-5-(1- | 617 |
| | piperazinylcarbonyl)phenyl]-N′-cyclohexylurea | |
| 48 | N-phenyl-N′-[2-[4-(1-phenylethyl)-1-piperazinyl]-5-(1- | 513 |
| | piperazinylcarbonyl)phenyl]-urea | |
| 49 | N-[2-[4-(2-methoxyphenyl)-1-piperazinyl]-5-(1-piperazinylcarbonyl)phenyl]- | 515 |
| | N′-phenylurea | |
| 50 | N-phenyl-N′-[2-[4-(phenylmethyl)-1-piperazinyl]-5-(1- | 499 |
| | piperazinylcarbonyl)phenyl]-urea | |
| 51 | N-[2-(4-cyclohexyl-1-piperazinyl)-5-(1-piperazinylcarbonyl)phenyl]- | 491 |
| | N′-phenylurea | |
| 52 | N-cyclohexyl-N′-[2-[4-(phenylmethyl)-1-piperazinyl]-5-(1- | 505 |
| | piperazinylcarbonyl)phenyl]-urea | |
| 53 | N-cyclohexyl-N′-[2-[4-(1-phenylethyl)-1-piperazinyl]-5-(1- | 519 |
| | piperazinylcarbonyl)phenyl]-urea | |
| 54 | N-cyclohexyl-N′-[2-(4-cyclohexyl-1-piperazinyl)-5-(1- | 497 |
| | piperazinylcarbonyl)phenyl]-urea | |
| 55 | N-cyclohexyl-N′-[2-[4-(2-methoxyphenyl)-1-piperazinyl]-5-(1- | 521 |
| | piperazinylcarbonyl)phenyl]-urea | |
| 56 | N-butyl-N′-[2-[4-(diphenylmethyl)-1-piperazinyl]-5-(1- | 555 |
| | piperazinylcarbonyl)phenyl]-urea | |
| 57 | N-[2-[4-(diphenylmethyl)-1-piperazinyl]-5-(1- | 593 |
| | piperazinylcarbonyl)phenyl]-N′-(2-fluorophenyl)urea | |
| 58 | N-[4-(dimethylamino)phenyl]-N′-[2-[4-(diphenylmethyl)-1- | 618 |
| | piperazinyl]-5-(1-piperazinylcarbonyl)phenyl]-urea | |
| 59 | N-[2-[4-(diphenylmethyl)-1-piperazinyl]-5-(1- | 605 |
| | piperazinylcarbonyl)phenyl]-N′-(2-methoxyphenyl)-urea | |
| 60 | N-[2-[4-[(4-chlorophenyl)phenylmethyl]-1-piperazinyl]-5-(1- | 610 |
| | piperazinylcarbonyl)phenyl]-N′-phenyl-urea | |
| 61 | N-[2-[4-(diphenylmethyl)-1-piperazinyl]-5-(1- | 629 |
| | piperazinylcarbonyl)phenyl]-N′-[(2E)-1-oxo-3-phenyl-2-propenyl]-urea | |
| 62 | N-(1,1-dimethylethyl)-N′-[2-[4-(diphenylmethyl)-1-piperazinyl]-5-(1- | 555 |
| | piperazinylcarbonyl)phenyl]-urea | |
| 63 | N-cyclopentyl-N′-[2-[4-(diphenylmethyl)-1-piperazinyl]-5-(1- | 567 |
| | piperazinylcarbonyl)phenyl]-urea | |
| 64 | N-[2-[4-(diphenylmethyl)-1-piperazinyl]-5-(1- | 603 |
| | piperazinylcarbonyl)phenyl]-N′-[(1S)-1-phenylethyl]-urea | |
| 65 | N-[2-[4-(diphenylmethyl)-1-piperazinyl]-5-(1- | 605 |
| | piperazinylcarbonyl)phenyl]-N′-(phenylmethyl)thiourea | |
| 66 | N-[2-[4-(diphenylmethyl)-1-piperazinyl]-5-(1-piperazinylcarbonyl)phenyl]- | 541 |
| | N′-(1-methylethyl)urea | |
| 67 | N-(4-chlorobenzoyl)-N′-[2-[4-(diphenylmethyl)-1-piperazinyl]-5-(1- | 654 |
| | piperazinylcarbonyl)phenyl]-thiourea | |

### EXAMPLE 3

### N-[4-[4-(diphenylmethyl)-1-piperazinyl]-3-[[(phenylamino)carbonyl]amino]benzoyl]-L-leucine (Cpd 32)

Commercially available Fmoc-Leu-Wang resin (1.0 g, 0.88 mmol) and a 40% piperidine:DMF (v/v) solution (10 mL) were added to a peptide reaction vessel. The mixture was shaken for 1 h using a wrist action shaker and the DMF was removed by vacuum filtration. The 40% piperidine/DMF solution (10 mL) was again added to the mixture while shaking for 30 min. The DMF was removed by vacuum filtration and the reaction product was sequentially washed with an excess of DMF, CH₂Cl₂ and MeOH, then a final wash with CH₂Cl₂ to provide a resin-bound amino acid Compound 3a was used in the next step without characterization.

A 4-fluoro-3-nitrobenzoic acid Compound 1b (2.31 g, 12.5 mmol) and 1-hydroxybenzotriazole (1.69 g, 12.5 mmol) were added in one portion to a 50 mL round bottom flask containing DMF (10 mL) and CH₂Cl₂ (10 mL) followed by 1,3-diisopropylcarbodiimide (1.95 mL, 12.5 mmol). The solution was then stirred for 30 min and added to the 50 mL reaction vessel containing Compound 3a (1.0 g, 0.88 mmol). The mixture was shaken for 16 h and the solvent was removed by vacuum filtration. The reaction product was sequentially washed with an excess of DMF, CH₂Cl₂ and MeOH, then a final wash with CH₂Cl₂ to provide a resin-bound 4-fluoro-3-nitro-benzamide Compound 3b. To characterize Compound 3b, an aliquot of the washed product (20 mg) was cleaved from the resin using 50% TFA in CH₂Cl₂ (1 mL), shaken for 1 h and filtered, then washed with MeOH and characterized: ESMS m/z 297 (M−H).

DMF (4 mL) and a 1-benzhydrylpiperazine Compound 1d (0.55 g, 2.2 mmol) were added to the reaction vessel containing Compound 3b (0.2 g, ˜0.2 mmol)then diisopropylethylamine (0.174 mL, 1 mmol) was added. The mixture was shaken over a 2 day period and turned from a pale yellow color to a yellow-orange color, then the solvent was removed by vacuum filtration. The reaction product was sequentially washed with an excess of DMF, CH₂Cl₂ and MeOH, then a final wash with CH₂Cl₂ to give a resin-bound nitro substituted piperazinylene benzamido Compound 3c. To characterize Compound 3c, an aliquot of the washed product (20 mg) was cleaved from the resin using 50% TFA in CH₂Cl₂ (1 mL), shaken for 1 h and filtered, then washed with MeOH and characterized: ESMS m/z 531 (M+H).

DMF (2 mL) and tin (II) chloride dihydrate (0.72 g, ˜3.2 mmol) were added to the reaction vessel containing Compound 3c (0.2 g, 0.1 mmol). The mixture was shaken overnight and turned from a yellow-orange color to almost colorless, then the solvent was removed by vacuum filtration. The reaction product was sequentially washed with excess of DMF, CH₂Cl₂ and MeOH, then a final wash with CH₂Cl₂ to give a resin-bound aminated Compound 3d. To characterize Compound 3d, an aliquot of the washed product (20 mg) was cleaved from the resin using 50% TFA in CH₂Cl₂ (1 mL), shaken for 1 h and filtered, then washed with MeOH and characterized: ESMS m/z 501 (M+H); m/z 499 (M−H).

Phenyl isocyanate (60 mg, 0.47 mmol) was added to the reaction vessel containing Compound 3d (0.06 g, ˜0.03 mmol) and CH₂Cl₂ (2 mL). The mixture was shaken for 48 h and the solvent was removed by vacuum filtration. The reaction product was sequentially washed with an excess of DMF, CH₂Cl₂ and MeOH, then a final wash with CH₂Cl₂ to give a resin-bound amino substituted Compound 3e. The washed Compound 3e was cleaved from the resin using 50% TFA in CH₂Cl₂ (1 mL), shaken for 1 h and filtered, then washed with MeOH. The filtrates were combined and concentrated to provide a crude trifluoroacetate salt. The salt was purified by column chromatography on silica gel (94:5:1 CH₂Cl₂:MeOH:acetic acid was used as the eluent) to provide Compound 32 (12 mg, 64% yield) as a pale yellow solid. ESMS m/z 620 (M+H); m/z 618 (M−H).

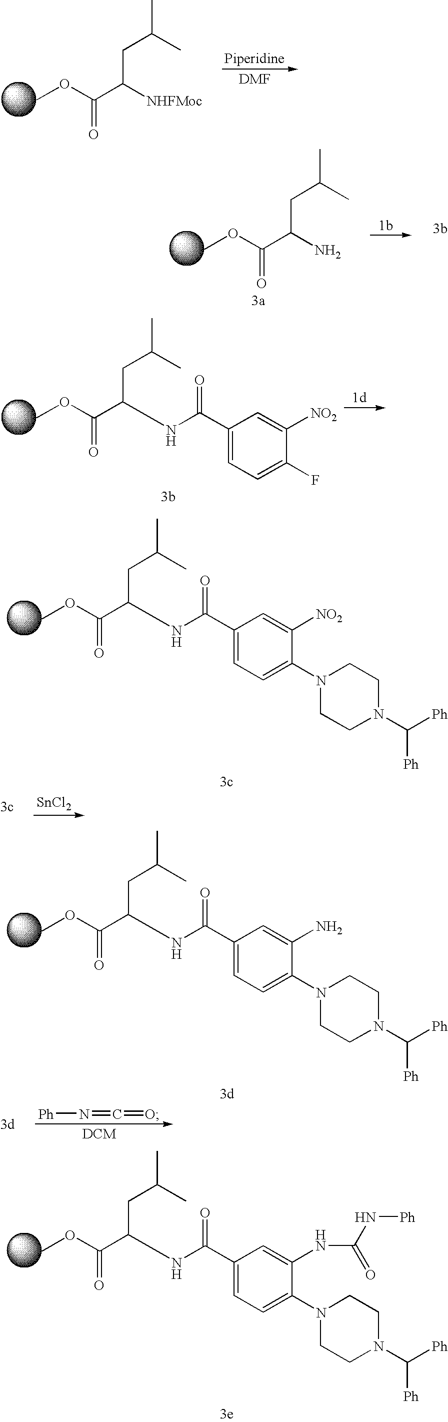

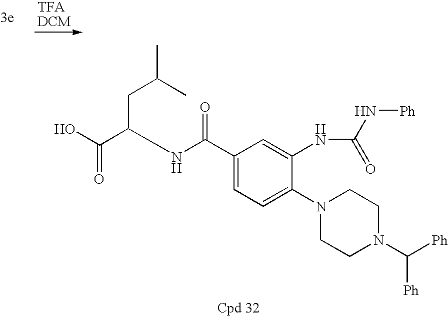

Solution Phase Synthesis of Regioisomeric Piperazinyl-piperazinoyl Substituted Anilino Compounds

Scheme C is an alternative to the solid phase synthesis methods of Scheme A and Scheme B. A commercially available t-BOC protected piperazine Compound Cl was coupled with a benzoic acid Compound A1 to yield Compound C₂.

The Compound C₂ fluoro atom was replaced with a Compound A3 (where n is preferably 1) to produce a piperazinyl-piperazinoyl substituted Compound C₃. The Compound C₃ nitro group was reduced to give corresponding piperazinyl-piperazinoyl substituted anilino Compound C₄. A compound such as an R₄—N═C═R₃ moiety was reacted with Compound C₄ to provide a Compound C₅. Deprotection of Compound C₅ yielded a Compound C₆ which was carried forward similarly to Compound B8 in Scheme B.

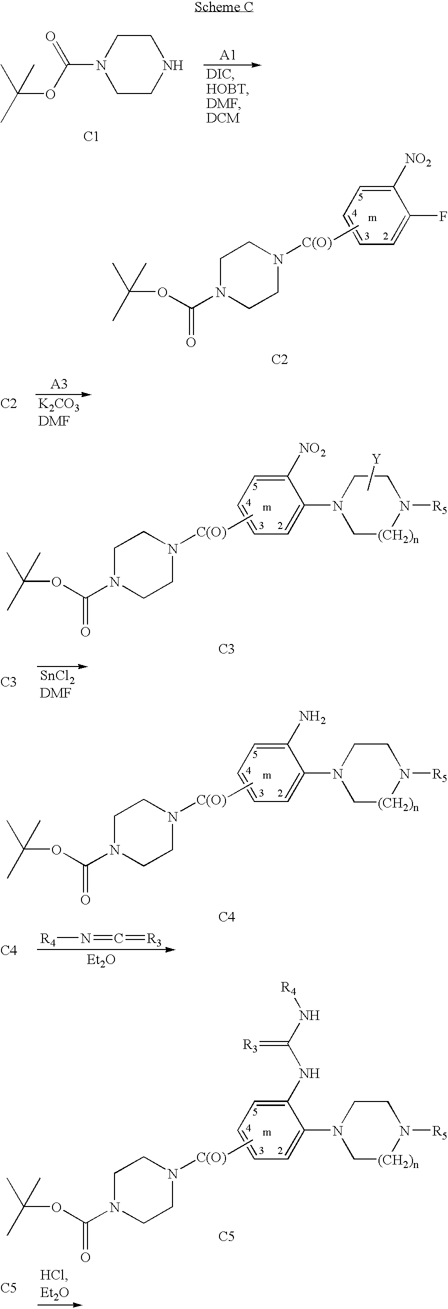

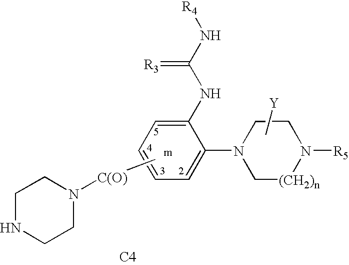

### EXAMPLE 4

### N-[2-[4-[bis(4-fluorophenyl)methyl]hexahydro-1H-1,4-diazepin-1-yl]-4-(1-piperazinylcarbonyl)phenyl]-N′-phenyl-urea (Cpd 89)

A 3-fluoro-4-nitro-benzoic acid Compound 4b (3.99 g, 21.55 mmol), 1,3-diisopropylcarbodiimide (2.72 g, 21.55 mmol) and 1-hydroxybenzotriazole (2.91 g, 21.55 mmol) were stirred in a mixture of DMF (50 mL) and DCM (50 mL) for 30 minutes. Piperazine-1-carboxylic acid tert-butyl ester Compound 4a (4.01 g, 21.55 mmol) was added and the solution was stirred for 18 h. The mixture was diluted with ethyl acetate (400 mL), washed with brine (2×125 mL) and water (3×300 mL), then dried over Na₂SO₄, filtered, concentrated in vacuo and purified by flash chromatography (silica gel, gradient of 0-5% MeOH in DCM) to give Compound 4c (7.74 g) as a yellow solid: ESMS m/z 354 (M⁺H).

A mixture of a commercially available [1,4]-diazepane-1-carboxylic acid tert-butyl ester Compound 4d (4.20 g, 20.95 mmol), chlorobis(4-fluorophenyl)methane Compound 4e (5.0 g, 20.95 mmol), potassium carbonate (4.34 g, 31.43 mmol) and acetonitrile (100 mL) was heated at 70° C. for 18 h. The mixture was cooled to rt, diluted with ethyl acetate (200 mL), washed with water (3×200 mL) and dried over Na₂SO₄, then filtered, concentrated in vacuo and purified by flash chromatography (silica gel, gradient of 0-25% ethyl acetate in hexane) to give an ester Compound 4f (4.39 g) as a colorless oil: ESMS m/z 303 (M⁺H).

A solution of Compound 4f (8.59 g, 21.34 mmol) and HCl in ether (1.0 M, 100 mL, 100 mmol) was stirred in methanol (5 mL) and ether (100 mL) for 18 h. The mixture was concentrated in vacuo, diluted with saturated sodium bicarbonate (250 mL) and extracted with DCM (2×150 mL). The combined organic layers were dried over Na₂SO₄, then filtered and concentrated in vacuo to afford an intermediate of Compound 4e (6.56 g) as a light brown oil: ESMS m/z 303 (M⁺H).

A mixture of the Compound 4f intermediate (1.28 g, 3.61 mmol), Compound 4c (1.20 g, 3.97 mmol), potassium carbonate (0.55 g, 3.97 mmol) and DMF (20 mL) was heated at 80° C. for 5 h. The mixture was cooled to rt, diluted with ethyl acetate (200 mL), washed with brine (2×100 mL) and water (2×200 mL), then dried over Na₂SO₄, filtered and concentrated in vacuo to afford Compound 4 g (2.38 g) as an orange foam: ESMS m/z 636 (M⁺H).

A solution of Compound 4 g (1.88 g, 2.96 mmol), tin (II) chloride dihydrate (6.67 g, 29.57 mmol) and DMF (30 mL) was stirred for 18 h. The reaction mixture was diluted with ethyl acetate (250 mL), washed with brine (2×150 mL) and water (5×200 mL), then dried over Na₂SO₄, filtered and concentrated in vacuo to afford Compound 4h (1.31 g) as a yellow foam: ESMS m/z 606 (M⁺H).

A mixture of Compound 4h (0.95 g, 1.57 mmol) and phenyl isocyanate (0.19 g, 1.57 mmol) in ether (20 mL) was stirred for 18 h. The precipitated reaction product was collected by filtration and washed with ether to afford Compound 4i (0.79 g) as a beige solid: ESMS m/z 725 (M⁺H). A solution of Compound 4i (0.79 g, 1.09 mmol), HCl in ether (1.0 M, 20 mL, 20 mmol), methanol (20 mL) and ether (20 mL) was stirred for 18 h. The reaction mixture was diluted with ethyl acetate (100 mL), washed with saturated sodium bicarbonate (2×75 mL) and dried over Na₂SO₄, then filtered, concentrated in vacuo and purified by flash chromatography (silica gel, gradient of 0-20% methanol in DCM) to afford Compound 89 (0.52 g) as a free base. The free base was dissolved in ethanol (10 mL) and ether (10 mL), then treated with HCl in ether (1.0 M, 20 mL, 20 mmol). Concentration gave Compound 89 as the corresponding dihydrochloride salt: ESMS m/z 625 (M⁺H).

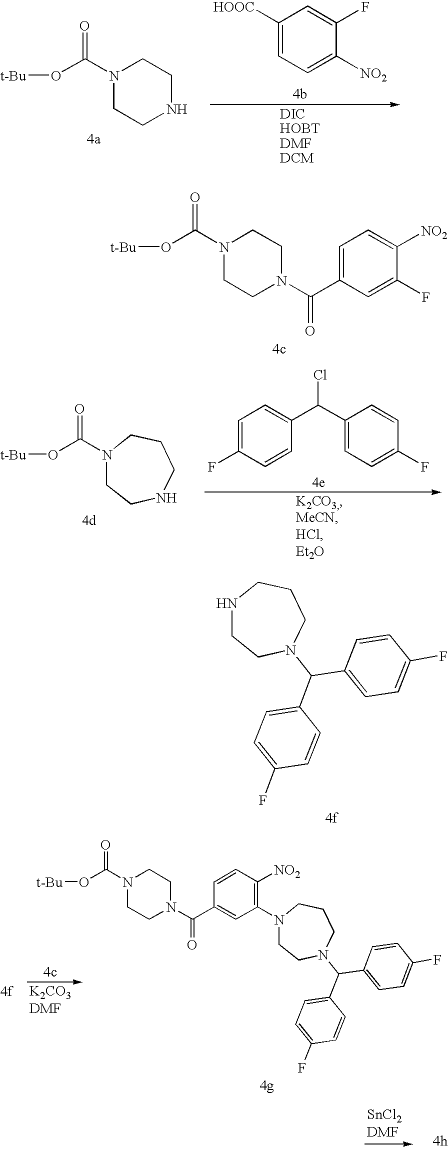

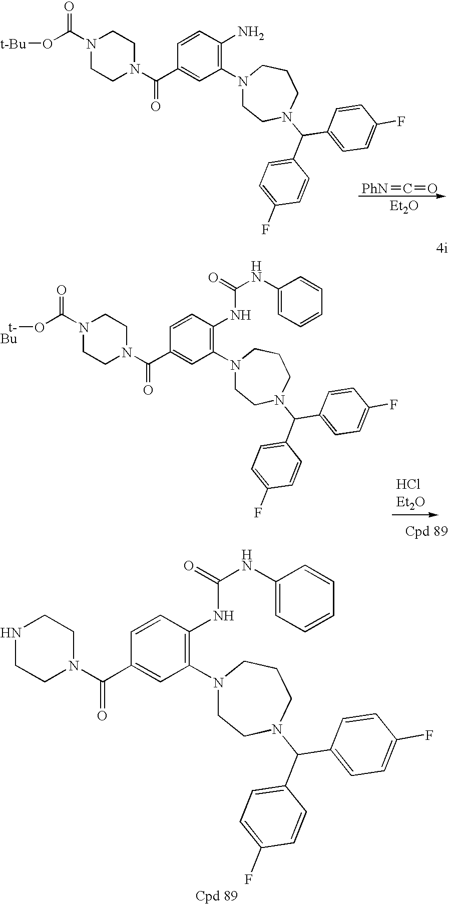

Using the procedure of Example 4 and the appropriate reagents and starting materials known to those skilled in the art, other compounds of the present invention may be prepared including, but not limited to (MS: Mass Spec data as MS m/z MH⁺):

| Cpd | Name | MS |
|---|---|---|
| 90 | N-[2-[4-[bis(4-fluorophenyl)methyl]-1-piperazinyl]-4-(1- | 611 |
| | piperazinylcarbonyl)phenyl]-N′-phenyl-urea | |
| 91 | N-[2-[4-(diphenylmethyl)-1-piperazinyl]-4-(1- | 575 |
| | piperazinylcarbonyl)phenyl]-N′-phenyl-urea | |
| 92 | N-cyclohexyl-N′-[2-[4-(diphenylmethyl)-1-piperazinyl]- | 581 |
| | 4-(1-piperazinylcarbonyl)phenyl]-urea | |

### EXAMPLE 5

### 3-[4-(diphenylmethyl)-1-piperazinyl]-4-[[(phenylamino)carbonyl]amino]-benzamide (Cpd 93)

Using the procedure of Example 1, commercially available Fmoc-Leu-Wang resin (3.47 g, 2.19 mmol) and a 40% piperidine:dimethylformamide (DMF) (v/v) solution (30 mL) were added to a peptide reaction vessel. The mixture was shaken for 2 h using a wrist action shaker and the DMF was removed by vacuum filtration. The 40% piperidine:DMF solution (30 mL) was again added to the mixture and shaken for 2 hr. The DMF was removed by vacuum filtration and the reaction product was sequentially washed with an excess of DMF, CH₂Cl₂ and MeOH, then a final wash with CH₂Cl₂ to provide a resin-bound amine Compound 1a used in the next step without characterization.

A 3-fluoro-4-nitrobenzoic acid Compound 4b (4.05 g, 21.86 mmol) and 1-hydroxybenzotriazole (HOBT) (2.95 g, 21.86 mmol) were added in one portion to a 50 mL round bottom flask containing DMF (15 mL) and CH₂Cl₂ (15 mL) followed by 1,3-diisopropylcarbodiimide (DIC) (3.5 mL, 21.86 mmol). The solution was stirred for 30 min and added to the 50 mL reaction vessel containing Compound 1a (3.47 g, 2.19 mmol). The mixture was shaken for 16 h and the solvent was removed by vacuum filtration. The reaction product was sequentially washed with an excess of DMF, CH₂Cl₂ and MeOH, then a final wash with CH₂Cl₂ to give a resin-bound benzamide Compound 5a. To characterize Compound 5a, an aliquot of the washed product (51 mg) was cleaved from the resin using 20% TFA in CH₂Cl₂ (1.2 mL), shaken for 1 h and filtered, then washed with MeOH and characterized: ESMS m/z 297 (M−H).

DMF (15 mL) and a 1-benzhydrylpiperazine Compound 1d (2.22 g, 8.8 mmol) were added to the reaction vessel containing Compound 5a (1.4 g, ˜0.88 mmol), then diisopropylethylamine (1.5 mL, 8.8 mmol) was added. The mixture was shaken over a 2 day period and turned from a pale yellow color to a yellow-orange color, then the solvent was removed by vacuum filtration. The reaction product was sequentially washed with an excess of DMF, CH₂Cl₂ and MeOH, then a final wash with CH₂Cl₂ to give a resin-bound piperazine substituted benzamide Compound 5b. To characterize Compound 5b, an aliquot of the washed product (60 mg) was cleaved from the resin using 20% TFA in CH₂Cl₂ (12 mL), shaken for 1 h and filtered, then washed with MeOH and characterized: ESMS m/z 417 (M+H).

DMF (15 mL) and tin (II) chloride dihydrate (3.97 g, 17.6 mmol) were added to the reaction vessel containing Compound 5b (˜0.88 mmol). The mixture was shaken over a 2 day period and turned from a yellow-orange color to almost colorless, then the solvent was removed by vacuum filtration. The reaction product was sequentially washed with an excess of DMF, CH₂Cl₂ and MeOH, then a final wash with CH₂Cl₂ to give a resin-bound aminated benzamido piperazine Compound 5c. To characterize Compound 5c, an aliquot of the washed product (38 mg) was cleaved from the resin using 20% TFA in CH₂Cl₂ (12 mL), shaken for 1 h and filtered, then washed with MeOH (0.5 mL)and characterized: ESMS m/z 387 (M+H).

Phenyl isocyanate (0.52 g, 4.4 mmol) was added to the reaction vessel containing Compound 5c (˜0.88 mmol) and CH₂Cl₂ (12 mL). The mixture was shaken for 24 h and the solvent was removed by vacuum filtration. The reaction product was sequentially washed with an excess of DMF, CH₂Cl₂ and MeOH, then a final wash with CH₂Cl₂ to give a resin-bound substituted amino benzamide Compound 5d. The washed Compound 5d was cleaved from the resin using 20% TFA in CH₂Cl₂ (12 mL), shaken for 1 h and filtered, then washed with MeOH. The filtrates were combined and concentrated to provide a crude trifluoroacetate salt. The salt was dissolved in CH₂Cl₂ (50 mL) and washed with saturated aqueous sodium bicarbonate (100 mL) to provide the free base. The organic layer was separated, dried over sodium sulfate and concentrated to give the free base as a beige solid. The hydrochloride salt was prepared by dissolving the free base in ether (5 mL) and adding 1.0 M hydrogen chloride in ether (5 mL). The solvent was removed and the salt dried under vacuum to give Compound 93 as a beige solid. ESMS m/z 506 (M+H).

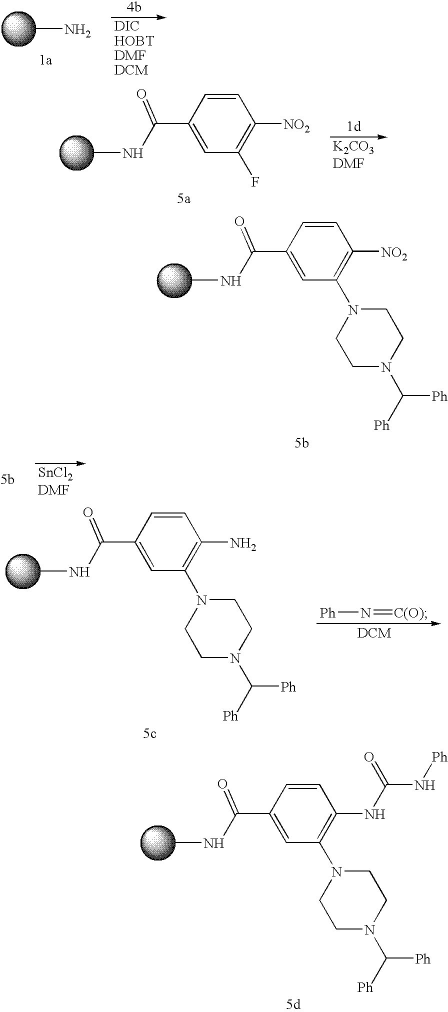

### BIOLOGICAL EXAMPLES

The compounds of the present invention are useful PLC-β2 inhibitors. The following biological example demonstrates that the PLC-β2 inhibitor compounds of the present invention are useful in the treatment or amelioration of diseases and conditions affected by the modulation of phospholipase, including the aformentioned inflammatory disorders.

### Example 1

The hydrolysis of phosphatidylinositol-4,5-bisphosphate (PIP₂) by a specific phospholipase C-β2 (PLC-β2) produces two intracellular messengers, diacylglycerol (DAG) and inositol 1,4,5-trisphosphate (IP₃), which mediate the activation of protein kinase C and intracellular Ca²⁺ release. A conventional organic solvent extraction method is widely used for PLC assays to isolate IP₃ from the substrate PIP₂. The conventional PLC-β2 assay, however is terminated by addition of acidified organic solvents and subsequent extraction and phase separation. The conventional method does not allow for validation of PLC-β2 assay on robots for the high throughput screening of PLC-β2 inhibitors. Accordingly, a preferred method to test the compounds of the present invention, was developed utilizing a 96-well plate assay for PLC-β2 using immobilized radiolabeled substrate to quantitatively measure the reduction in the substrate level without a need for organic solvent extraction. The automated PLC-β2 assay described herein provides a convenient method for quantitative measurement of phospholipase C activities in a high throughput fashion.

Materials

Phospholipid FlashPlates and [³H]PIP₂ (20 Ci/mmol) were purchased from NEN Life Science Products (Boston, Mass. USA). BSA (acetylated), fatty acid-free BSA, sodium chloride, potassium chloride, PMSF, benzamidine, pepstatin A, calcium chloride, HEPES, and sodium deoxycholate were purchased from Sigma Chemical Co. (St. Louis, Mo. USA). DTT was purchased from Boehringer Mannheim (Indianapolis, Ind. USA). Q-Sepharose FF, Heparin-Sepharose CL-6B, and the Mono Q HR 5/5 column were purchased from Amersham-Pharmacia (Piscataway, N.J. USA). Bio-Gel HPHT column and Bio-Gel HPHT were from Bio-Rad Laboratories (Hercules, Calif. USA). HL-60 and Sf9 cells from spodopterafrugiperda (ATCC CRL-171 1) were purchased from ATCC (Rockville, Md. USA). All other reagents were obtained from readily available commercial sources.

PLC Assay Using FlashPlates

Ninety-six well Phospholipid FlashPlates were coated with 0.2 mL of 50 mM Tris/HCl (pH 7.4), 0.01% Ac-BSA and 50,000 cpm of [³H]PrP₂ (phosphatidylinositol-4,5-bisphosphate) at 4° C. for 72 h. The wells were aspirated and washed twice with PBS. The reactions were conducted directly in the wells in PLC reaction buffer containing 50 mM Tris/HCl (pH 7.2), 2.75 mM EDTA (pH 7.3), 80 mM KCl, 10 mM LiCl, 0.04% DOC and 2 mM CaCl₂ in the absence or presence of the purified recombinant human PLC-β2 (prepared as described hereafter) or cytosolic human PLC-β2 from JL-60 cells. Reduction of radioactivity was monitored by a Packard TopCount instrument (Packard Instrument Company, Conn., USA).

Production of Recombinant PLC-β2 in Sf9 Cells

Suspension cultures of Sf9 cells were maintained in a spinner flask at 27° C. and stirred at 90 rpm. The cells were grown in Grace's media supplemented with 10% (v/v) fetal bovine serum, 3.3 g/l yeastolate, 3.3 g/l lactalbumin hydrosylate, glutamine (6.4 mM final), 50 μg/ml gentamicin, and 50 μg/ml kanamycin. Suspension of Sf9 cells (1.0×106 cells/ml) were infected with 5 pfu/cell of recombinant baculovirus encoding PLC-β2 and incubated at 27° C. for 72 h. The cells were collected by centrifugation (500×g, 7 min, 4° C.) and disrupted by hypotonic lysis buffer containing 20 mM Tris/HCl, pH 7.4, 5 mM MgCl₂, 2 mM EGTA, 200 μM PMSF, 200 μM benzamidine and 1 μM pepstatin A. The lysate was sonicated on ice and the nuclei and unbroken cells removed by centrifigation (500×g, 5 min, 4C.). The supernatant was recovered and clarified by centrifugation (34,000 rpm, 60 min, 4C.). The supernatant was used as a crude cytosolic fraction (Paterson, A., Boyer, J. L., Watts, V. J., Morris, A. J., Price, E. M., Harden, T. K. (1995) Concentration of enzyme-dependent activation of PLC β1 and PLC β2 by Gα₁₁ and βγ subunits. Cellular Signalling 7, 709-720).

Purification of recombinant PLC-β2

Crude cytosol prepared from Sf9 cells expressing PLC-β2 was purified initially by chromatography on a 10 ml column of Q-Sepharose FF, equilibrated in buffer A (25 mM HEPES, pH 7.2,2 mM DTT, 2 mM EDTA, 2 mM EGTA, 200 μM PMSF, 200 μM benzamidine, 1 μM pepstatin A containing 10 mM NaCl). The column was washed with 20 ml of equilibration buffer and eluted with a 200-ml gradient of 110-410 mM NaCl in buffer A. The fractions containing PLC activity were pooled and diluted with buffer A. The diluted enzyme was applied to a 4 ml column of heparin-SepharoseCL-6B equilibrated in buffer A and the column washed with 70 ml of buffer A. The column was eluted with 80 ml of gradient of 0-1.0 M NaCl in buffer A, the column eluate collected in 3 ml fractions. The fractions containing PLC activity were pooled and diluted in buffer B (25 mM HEPES pH 7.2, 10 mM KCl, 2 mM DTT, 200 μM PMSF, 200 μM benzamidine, 1 μM pepstatin A) and applied to a Bio-Gel HPHT (10 ml) hydroxylapatite column operated in conjunction with a Bio-Gel HPHT and equilibrated in buffer B. The column was washed with 20 ml of buffer B and PLC-β2 eluted with a gradient of 0-500 mM potassium phosphate in buffer B. The fractions containing PLC activity were pooled, diluted with buffer A containing 10 mM NaCl and applied to an FPLC Mono Q HR 5/5 column equilibrated in buffer A. The column was washed with 5.0 ml of equilibration buffer and then eluted with a 10 ml gradient of 0.01-1.0 M NaCl in buffer A. The column eluate was collected in 0.5 ml fractions. The fractions containing PLC activity were pooled and diluted in buffer A containing 20% glycerol and stored at −80° C.

Cell Culture and Preparation of Cytosolic PLC

HL-60 cells were grown in suspension and induced to differentiate into mature myeloid forms by addition of 1.25% (v/v) DMSO to the culture medium. Differentiated cells were pelleted by centrifugation, resuspended in 200 ml of lysis buffer containing 250 mM sucrose, 20 mM.Tris-HCl, pH 7.5, 1.5 mM MgCl₂, 1 mM ATP, 3 mM benzamidine, 1 μM leupeptin, 1 mM PMSF and 2 μg/ml of soybean trypsin inhibitor (Camps, M., Hou, C., Jakobs, K. H., and Gierschik, P. (1990) Guanosine 5′-[y-thio]triphosphate-stimulated hydrolysis of phosphatidylinositol 4,5-bisphosphate in HL-60 granulocytes. Biochem. J. 271, 743-748). Cells were homogenized by nitrogen cavitation. Cytosol was prepared from the post-nuclear supernatant by sequential centrifugation. In some cases, cytosol was concentrated by pressure filtration in a stirred cell equipped with an Amicon PM 10 membrane.

Purification of βγ Subunits of Retinal Transducin

Retinal rod outer segment membranes were prepared from bovine eyes as described in Camps, M., Hou, C., Sidroupoulos, D., Stock, J. B., Jakobs, K. H., Gierschik, P., (1992) Stimulation of phospholipase C by guanine-nucleotide-binding protein βγ subunits. Eur. J. Biochem. 206, 821-831. Transducin was eluted from the membranes with buffer containing 100 μM GTP and used for the subunit preparation procedure without delay. Transducin was resolved into αₜ and βγₜ subunits by chromatography on Blue Sepharose CL-6B using a FPLC equipment (Pharmacia). Fractions containing βγₜ subunits were pooled and concentrated about 20-fold by centrifugation using a CentriCon 10 PM (Amicon). The purified protein was snap-frozen in liquid nitrogen and stored at −80° C.

Results

The results for compounds of the present invention are shown in the following table:

| Cpd | IC₅₀(μM) |
|---|---|
| 1 | 23.4 |
| 2 | >25 |
| 3 | 1.5 |
| 4 | 2.1 |
| 5 | 9.9 |
| 6 | 9.0 |
| 7 | 5.3 |
| 8 | 1.2 |
| 9 | 1.9 |
| 10 | 14.0 |
| 11 | 8.4 |
| 12 | 14.5 |
| 13 | 1.3 |
| 14 | 3.1 |
| 15 | 4.8 |
| 16 | 6.0 |
| 17 | 2.8 |
| 18 | 4.6 |
| 19 | 2.9 |
| 20 | 4.3 |
| 21 | 2.3 |
| 22 | 1.2 |
| 23 | 4.9 |
| 24 | 0.87 |
| 25 | 2.6 |
| 26 | 1.9 |
| 27 | 2.8 |
| 28 | 5.6 |
| 29 | 3.3 |
| 30 | 9.8 |
| 31 | 6.6 |
| 32 | 3.9 |
| 38 | 2.2 |
| 39 | 4.2 |
| 40 | 1.6 |
| 41 | 1.6 |
| 42 | 5.6 |
| 43 | 5.7 |
| 44 | 2.3 |
| 45 | 2.2 |
| 46 | 1.7 |
| 47 | 4.2 |
| 48 | >25 |
| 49 | >25 |
| 50 | >25 |
| 51 | >25 |
| 52 | >25 |
| 53 | >25 |
| 54 | >25 |
| 55 | >25 |
| 56 | >25 |
| 57 | 2.5 |
| 58 | 7.8 |
| 59 | 5.4 |
| 60 | 0.95 |
| 61 | 4.3 |
| 62 | >25 |
| 63 | 13.7 |
| 64 | >25 |
| 65 | 6.2 |
| 66 | >25 |
| 67 | 3.4 |
| 77 | ˜10 |
| 78 | >10 |
| 79 | >10 |
| 80 | >10 |
| 81 | ˜10 |
| 82 | ˜10 |
| 83 | ˜10 |
| 89 | 0.81 |
| 90 | 0.98 |
| 91 | 1.3 |
| 92 | 1.6 |
| 93 | 1.8 |

### Example 2

Acute PMA-induced Ear Edema Mouse Model

The acute PMA-induced ear edema mouse model was used to test compounds of the present vision (as described in Carlson R P, O'Neill-Davis L, Chang J. and Lewis A J, Modulation of Mouse Ear Edema by Cyclooxygenase and Lipoxygenase Inhibitors and Other Pharmacologic Agents, Agents and Actions, 1985, 17:197-204). As shown in the following table, in vivo results in % inhibition demonstrated at various mpk (milligrams per kilogram) doses administered i.p. (intraperitoneally), p.o. (orally) or i.v. (intravenously) hat certain compounds of the present invention are PLC-β2 inhibitors and, depending on the route of administration, are useful in a method for treating or ameloriating an inflammatory disorder.

| Cpd | Administration | % Inhibition |
|---|---|---|
| 9 | 30 mpk i.p. | 52 |
| | 30 mpk p.o. | 44 |
| | 5 mpk i.v. | 44 |
| 21 | 30 mpk i.p. | 83 |
| | 30 mpk p.o. | 31 |
| | 5 mpk i.v. | 56 |
| 38 | 30 mpk i.p. | 84 |
| | 30 mpk p.o. | 0 |
| | 5 mpk i.v. | 47 |
| 40 | 30 mpk i.p. | 81 |
| | 30 mpk p.o. | 9 |
| | 5 mpk i.v. | 44 |

### Example 3

Chronic PMA-induced Ear Edema Mouse Model

The chronic PMA-induced ear edema mouse model was used to test Compound 38 (as described in Stanley P L, Steiner S, Havens M and Tramposch K M, Mouse Skin Inflammation Induced by Multiple Topical Applications of 12-O-Tetradecanoylphorbol-13-acetate, Skin Pharmacol., 1991, 4: 262-271). An in vivo result of 53% inhibition at a 0.5 mg dose (applied topically) was demonstrated.

### Example 4

Zymosan-induced Peritonitis Mouse Model

The zymosan-induced peritonitis model was used to test Compound 38 (as described in Rao T S, Currie J L, Shaffer A F and Isakson P C, In vivo Characterization of Zymosan-Induced Mouse Peritoneal Inflammation, J. Pharm. Exptl. Ther., 1994, 269: 917-925). An in vivo result of about 45% inhibition at a 30 mpk dose (administered i.p.) was demonstrated.

### Example 5

Adjuvant-induced Arthritis Rat Model

The adjuvant-induced arthritis rat model was used to test Compound 38 (as described in Anderson G D, Hauser S D, McGarity K L, Bremer M E, Isakson P C and Gregory S A, Selective Inhibition of Cyclooxygenase (Cox)-2 Reverses Inflammation and Expression of Cox-2 and Interleukein 6 in Rat Adjuvant Arthritis, J. Clin. Invest., 1996, 97:2672-2679). An in vivo result showing significant inhibition in both paws (administered i.p.) was demonstrated.

### Example 6

Carageenan-induced Paw Edema Rat Model

The carageenan-induced paw edema rat model was used to test Compound 38 (as described in Vinegar R, Truax J F, Selph J L, Johnston P R, Venable A L and McKenzie K K, Pathway to Carrageenan-Induced Inflammation in the Hind Limb of the Rat, Fed. Proc., 1987, 46:118-126). In vivo results of 60% inhibition at a 30 mpk dose (administered s.c.) and 80% inhibition at a 30 mpk dose (administered i.p.) were demonstrated.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A compound of formula (I): 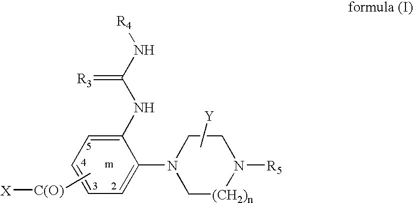
and enantiomers, diastereomers and pharmaceutically acceptable salts thereof, wherein:
X—C(O)— is a substituent moiety having a variable position “m”, wherein “m” represents a carbon atom number corresponding to a point of attachment for the X—C(O)— substituent moiety on the anilino ring of formula (I);
X is selected from the group consisting of
(i) R₁—NH— (amino optionally substituted with R₁); and,
(ii) a heterocyclyl ring optionally substituted with R₂, said heterocyclyl ring having at least one nitrogen atom member, wherein the nitrogen atom member forms the point of attachment for said heterocyclyl ring on the —C(O)— portion of X—C(O)—;
R₁ and R₂ are independently selected from the group consisting of hydrogen and C₁₋₈alkyl, wherein C₁₋₈alkyl is optionally substituted with one or more substituents independently selected from the group consisting of amino, mono(C₁₋₈)alkylamino, di(C₁₋₈)alkylamino, cyano, halogen, hydroxy, nitro and carboxyl;
R₃ is selected from the group consisting of O and S;
R₄ is selected from the group consisting of
(a) C₁₋₈alkyl optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(b) carbonyl(C₁₋₈)alkyl, wherein the C₁₋₈alkyl portion of the carbonyl(C₁₋₈)alkyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₄)alkylamino, di(C₁₋₄)alkylamino, cyano, halogen, hydroxy and nitro;
(c) carbonyl(C₂₋₈)alkenyl, wherein the C₂₋₈alkenyl portion of the carbonyl(C₂₋₈)alkenyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(d) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(e) benzofused dioxolyl;
(f) benzofused dioxinyl;
(g) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(h) carbonyl-aryl, wherein the aryl portion of the carbonyl-aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
R₅ is one substituent selected from the group consisting of
(i) C₁₋₈alkyl optionally substituted with one or more substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,
wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
wherein said heteroaryl is optionally substituted on a secondary amine atom with C₁₋₈alkyl, and optionally and independently substituted on a carbon atom with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(j) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
(k) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
Y is one or more optionally present C₁₋₈alkyl substituents optionally substituted with one or more substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;
m is an integer from 2 to 5 which represents the carbon atom number corresponding to the point of attachment for the X—C(O)— substituent moiety on the anilino ring of formula (I); and, n is an integer from 1 to 2.

2. The compound of claim 1, wherein when X is R₁—NH—, R₁ is hydrogen and R₄ is C₁₋₈alkyl, then R₄ is substituted C₁₋₈alkyl.

3. The compound of claim 1, wherein when R₄ is unsubstituted C₁₋₈alkyl, then X is a heterocyclyl ring optionally substituted with R₂.

4. The compound of claim 1, wherein when R₄ is optionally substituted C₁₋₈alkyl, then R₅ is C₁₋₈alkyl substituted on one or more carbon atoms with one or more optionally substituted aryl substituents.

5. The compound of claim 1, wherein 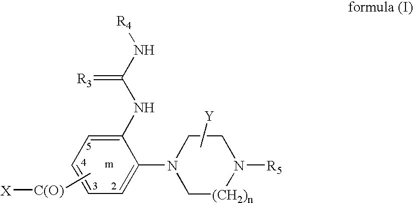
and enantiomers, diastereomers and pharmaceutically acceptable salts thereof, wherein:
X—C(O)— is a substituent moiety having a variable position “m”, wherein “m” represents a carbon atom number corresponding to a point of attachment for the X—C(O)— substituent moiety on the anilino ring of formula (I);
X is selected from the group consisting of
(i) R₁—NH— (amino optionally substituted with R₁); and,
(ii) a heterocyclyl ring optionally substituted with R₂, said heterocyclyl ring having at least one nitrogen atom member, wherein the nitrogen atom member forms the point of attachment for said heterocyclyl ring on the —C(O)— portion of X—C(O)—;
R₁ and R₂ are independently selected from the group consisting of hydrogen and C₁₋₈alkyl, wherein C₁₋₈alkyl is optionally substituted with one or two substituents independently selected from the group consisting of amino, mono(C₁₋₈)alkylamino, di(C₁₋₈)alkylamino, cyano, halogen, hydroxy, nitro and carboxyl;
R₃ is selected from the group consisting of O and S;
R₄ is selected from the group consisting of
(a) C₁₋₈alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(b) carbonyl(C₁₋₈)alkyl, wherein the C₁₋₈alkyl portion of the carbonyl(C₁₋₈)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(c) carbonyl(C₂₋₈)alkenyl, wherein the C₂₋₈alkenyl portion of the carbonyl(C₂₋₈)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(d) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(e) benzofused dioxolyl;
(f) benzofused dioxinyl;
(g) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(h) carbonyl-aryl, wherein the aryl portion of the carbonyl-aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
R₅ is one substituent selected from the group consisting of
(i) C₁₋₈alkyl optionally substituted with one or two substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,
wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
wherein said heteroaryl is optionally substituted on a secondary amine atom with C₁₋₈alkyl, and optionally and independently substituted on a carbon atom with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(j) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
(k) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
Y is one or two optionally present C₁₋₈alkyl substituents optionally substituted with one or two substituents independently selected from the group consisting of amino, mono(C₁₋₄)alkylanino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;
m is an integer from 2 to 5 which represents the carbon atom number corresponding to the point of attachment for the X—C(O)— substituent moiety on the anilino ring of formula (I); and, n is an integer from 1 to 2.

6. The compound of claim 5, wherein when X is R₁—NH—, R₁ is hydrogen and R₄ is C₁₋₈alkyl, then R₄ is substituted C₁₋₈alkyl.

7. The compound of claim 5, wherein when R₄ is unsubstituted C₁₋₈alkyl, then X is a heterocyclyl ring optionally substituted with R₂.

8. The compound of claim 5, wherein when R₄ is optionally substituted C₁₋₈alkyl, then R₅ is C₁₋₈alkyl substituted on one or two carbon atoms with one or two optionally substituted aryl substituents.

9. The compound of claim 1, wherein R₁ is selected from the group consisting of hydrogen and C₁₋₈alkyl, wherein C₁₋₈alkyl is optionally substituted with one or two substituents independently selected from the group consisting of amino, mono(C₁₋₈)alkylamino, di(C₁₋₈)alkylamino and carboxyl.

10. The compound of claim 1, wherein R₂ is selected from the group consisting of hydrogen and C₁₋₆alkyl, wherein C₁₋₆alkyl is optionally substituted with one or two substituents independently selected from the group consisting of amino, mono(C₁₋₄)alkylamino, di(C₁₋₄)alkylamino and carboxyl.

11. The compound of claim 1, wherein R₄ is selected from the group consisting of
(a) C₁₋₄alkyl optionally substituted with one aryl substituent, wherein said aryl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;
(c) carbonyl(C₂₋₄)alkenyl, wherein the C₂₋₄alkenyl portion of the carbonyl(C₂₋₄)alkenyl is substituted with one phenyl substituent, wherein said phenyl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;
(d) C₃₋₈cycloalkyl;
(e) benzofused dioxolyl;
(f) benzoftised dioxinyl;
(g) aryl optionally substituted with one or two substituents independently selected from the group consisting of C₁₋₄alkyl, C₁₋₄alkoxy, mono(C₁₋₄)alkylamino, di(C₁₋₄)alkylamino, halogen and nitro; and,
(h) carbonyl-phenyl, wherein the phenyl portion of the carbonyl-phenyl is optionally substituted with one substituent selected from the group consisting of C₁₋₄alkyl, C₁₋₄alkoxy, amino, mono(C₁₋₄)alkylamino, di(C₁₋₄)alkylamino, cyano, halogen, hydroxy and nitro.

12. The compound of claim 1, wherein R₄ is selected from the group consisting of
(a) C₁₋₄alkyl optionally substituted with one phenyl substituent, wherein said phenyl is optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;
(c) carbonyl(C₂₋₄)alkenyl, wherein the C₂₋₄alkenyl portion of the carbonyl(C₂₋₄)alkenyl is substituted with one phenyl substituent;
(d) C₃₋₈cycloalkyl;
(e) benzofused dioxolyl;
(f) benzofused dioxinyl;
(g) phenyl optionally substituted with one or two substituents independently selected from the group consisting of C₁₋₄alkyl, C₁₋₄alkoxy, di(C₁₋₄)alkylamino, halogen and nitro; and,
(h) carbonyl-phenyl, wherein the phenyl portion of the carbonyl-phenyl is optionally substituted with one halogen substituent.

13. The compound of claim 1, wherein R₄ is selected from the group consisting of
(a) C₁₋₄alkyl optionally substituted with one phenyl substituent;
(c) carbonyl(C₂₋₄)alkenyl, wherein the C₂₋₄alkenyl portion of the carbonyl(C₂₋₄)alkenyl is substituted with one phenyl substituent;
(d) C₅₋₆cycloalkyl;
(e) 1,3-benzodioxol-5-yl;
(f) 2,3-dihydro-1,4-benzodioxinyl;
(g) phenyl optionally substituted with one or two substituents independently selected from the group consisting of C₁₋₄alkyl, C₁₋₄alkoxy, di(C₁₋₄)alkylamino, chlorine, fluorine and nitro; and,
(h) carbonyl-phenyl, wherein the phenyl portion of the carbonyl-phenyl is optionally substituted with one chlorine substituent.

14. The compound of claim 1, wherein R₅ is one substituent selected from the group consisting of
(i) C₁₋₄alkyl optionally substituted with one or two substituents independently selected from the group consisting of C₁₋₄alkoxy, amino, mono(C₁₋₄)alkylamino, di(C₁₋₄)alkylamino, cyano, halogen, hydroxy, nitro, aryl and heteroaryl, wherein said aryl is optionally substituted one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro;
(j) C₃₋₈cycloalkyl; and,
(k) aryl optionally substituted with one or two substituents independently selected from the group consisting of C₁₋₄alkyl, C₁₋₄alkoxy, amino, mono(C₁₋₄)alkylamino, di(C₁₋₄)alkylamino, cyano, halogen, hydroxy and nitro.

15. The compound of claim 1, wherein R₅ is one substituent selected from the group consisting of
(i) C₁₋₄alkyl optionally substituted with one or two substituents independently selected from the group consisting of phenyl and pyridinyl; wherein said phenyl is optionally substituted with one chlorine or one fluorine substituent;
(j) cyclohexyl; and,
(k) fluorenyl or phenyl, wherein said phenyl is optionally substituted with one C₁₋₄alkoxy substituent.

16. The compound of claim 1, wherein X is selected from the group consisting of
(i) R₁—NH— wherein R₁ is selected from the group consisting of hydrogen and C₁₋₈alkyl, wherein C₁₋₈alkyl is optionally substituted with one or two substituents independently selected from the group consisting of amino, mono(C₁₋₄)alkylamino, di(C₁₋₄)alkylamino, cyano, halogen, hydroxy, nitro and carboxyl; and,
(ii) a heterocyclyl ring selected from the group consisting of piperazinyl and hexahydro-1H-1,4-diazepinyl optionally'substituted with R₂, wherein one piperazinyl and hexahydro-1H-1,4-diazepinyl ring nitrogen atom member forms the point of attachment for said ring on the —C(O)— portion of X—C(O)—, wherein R₂ is selected from the group consisting of hydrogen and C₁₋₈alkyl;
R₃ is selected from the group consisting of O and S;
R₄ is selected from the group consisting of
(a) C₁₋₈alkyl optionally substituted with aryl;
(c) carbonyl(C₂₋₈)alkenyl, wherein the C₂₋₈alkenyl portion of the carbonyl(C₂₋₈)alkenyl is substituted with aryl;
(d) C₃₋₈cycloalkyl;
(e) 1,3-benzodioxol-5-yl;
(g) aryl optionally substituted with one or two substituents independently selected from the group consisting of C₁₋₈alkyl, C₁₋₈alkoxy, di(C₁₋₄)alkylamino, halogen and nitro; and,
(h) carbonyl-aryl, wherein the aryl portion of the carbonyl-aryl is optionally substituted with one halogen substituent;
R₅ is one substituent selected from the group consisting of
(i) C₁₋₈alkyl substituted with one or two substituents independently selected from the group consisting of aryl and heteroaryl, wherein said aryl is optionally substituted with one halogen substituent;
(j) C₃₋₈cycloalkyl; and,
(k) aryl optionally substituted with one C₁₋₈alkoxy substituent;
m is an integer from 2 to 5 which represents the carbon atom number corresponding to the point of attachment for the X—C(O)— substituent moiety on the anilino ring of formula (I); and, n is an integer from 1 to 2.

17. The compound of claim 16, wherein
R₁ is selected from the group consisting of hydrogen and C₁₋₈alkyl, wherein C₁₋₈alkyl is optionally substituted with one substituent independently selected from the group consisting of amino, mono(C₁₋₈)alkylamino, di(C₁₋₈)alkylamino and carboxyl;
R₂ is selected from the group consisting of hydrogen and C₁₋₆alkyl;
R₄ is selected from the group consisting of
(a) C₁₋₄alkyl optionally substituted with one phenyl substituent;
(c) carbonyl(C₂₋₄)alkenyl, wherein the C₂₋₄alkenyl portion of the carbonyl(C₂₋₄)alkenyl is substituted with one phenyl substituent;
(d) C₅₋₆cycloalkyl;
(e) 1,3-benzodioxol-5-yl;
(f) 2,3-dihydro-1,4-benzodioxinyl;
(g) phenyl optionally substituted with one or two substituents independently selected from the group consisting of C₁₋₄alkyl, C₁₋₄alkoxy, di(C₁₋₄)alkylamino, chlorine, fluorine and nitro; and,
(h) carbonyl-phenyl, wherein the phenyl portion of the carbonyl-phenyl is optionally substituted with one chlorine substituent;
R₅ is one substituent selected from the group consisting of
(i) C₁₋₄alkyl optionally substituted with one or two substituents independently selected from the group consisting of phenyl and pyridinyl; wherein said phenyl is optionally substituted with one chlorine or one fluorine substituent;
(j) cyclohexyl; and,
(k) fluorenyl or phenyl, wherein said phenyl is optionally substituted with one C₁₋₄alkoxy substituent;
m is an integer from 3 to 4 which represents the carbon atom number corresponding to the point of attachment for the X—C(O)— substituent moiety on the anilino ring of formula (I); and, n is 1.

18. The compound of claim 1, wherein the compound of formula (I) is selected from a compound of formula (Ia): 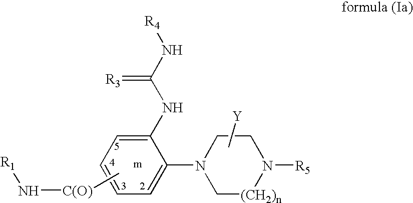
and enantiomers, diastereomers and pharmaceutically acceptable salts thereof, wherein:
R₁—NH—C(O)— is a substituent moiety having a variable position “m”, wherein “m” represents a carbon atom number corresponding to a point of attachment for the R₁—NH—C(O)— substituent moiety on the anilino ring of formula (Ia);
R₁ is selected from the group consisting of hydrogen and C₁₋₈alkyl, wherein C₁₋₈alkyl is optionally substituted with one or more substituents independently selected from the group consisting of amino, mono(C₁₋₈)alkylamino, di(C₁₋₈)alkylamino, cyano, halogen, hydroxy, nitro and carboxyl;
R₃ is selected from the group consisting of O and S;
R₄ is selected from the group consisting of
(a) C₁₋₈alkyl optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(b) carbonyl(C₁₋₈)alkyl, wherein the C₁₋₈alkyl portion of the carbonyl(C₁₋₈)alkyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(c) carbonyl(C₂₋₈)alkenyl, wherein the C₂₋₈alkenyl portion of the carbonyl(C₂₋₈)alkenyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(d) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(e) benzofused dioxolyl;
(f) benzofused dioxinyl;
(g) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(h) carbonyl-aryl, wherein the aryl portion of the carbonyl-aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
R₅ is one substituent selected from the group consisting of
(i) C₁₋₈alkyl optionally substituted with one or more substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,
wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
wherein said heteroaryl is optionally substituted on a secondary amine atom with C₁₋₈alkyl, and optionally and independently substituted on a carbon atom with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(j) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
(k) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
Y is one or more optionally present C₁₋₈alkyl substituents optionally substituted with one or more substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;
m is an integer from 2 to 5 which represents the carbon atom number corresponding to the point of attachment for the R₁—NH—C(O)-substituent moiety on the anilino ring of formula (Ia); and, n is an integer from 1 to 2.

19. The compound of claim 18, wherein when the R₁—NH—C(O)— substituent moiety is NH₂—C(O)— and R₄ is C₁₋₈alkyl, then R₄ is substituted C₁₋₈alkyl.

20. The compound of claim 18, wherein when R₄ is optionally substituted C₁₋₈alkyl, then R₅ is C₁₋₈alkyl substituted on one or more carbon atoms with one or more optionally substituted aryl substituents.

21. The compound of claim 1, wherein the compound of formula (I) is selected from a compound of formula (Ia): 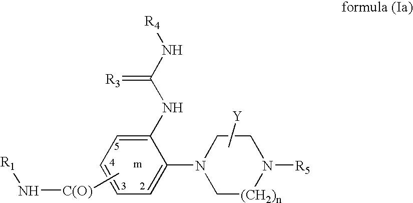
and enantiomers, diastereomers and pharmaceutically acceptable salts thereof, wherein:
R₁—NH—C(O)— is a substituent moiety having a variable position “m”, wherein “m” represents a carbon atom number corresponding to a point of attachment for the R₁—NH—C(O)— substituent moiety on the anilino ring of formula (Ia);
R₁ is selected from the group consisting of hydrogen and C₁₋₈alkyl, wherein C₁₋₈alkyl is optionally substituted with one or two substituents independently selected from the group consisting of amino, mono(C₁₋₈)alkylamino, di(C₁₋₈)alkylamino, cyano, halogen, hydroxy, nitro and carboxyl;
R₃ is selected from the group consisting of O and S;
R₄ is selected from the group consisting of
(a) C₁₋₈alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(b) carbonyl(C₁₋₈)alkyl, wherein the C₁₋₈alkyl portion of the carbonyl(C₁₋₈)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(c) carbonyl(C₂₋₈)alkenyl, wherein the C₂₋₈alkenyl portion of the carbonyl(C₂₋₈)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(d) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(e) benzofused dioxolyl;
(f) benzofused dioxinyl;
(g) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(h) carbonyl-aryl, wherein the aryl portion of the carbonyl-aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
R₅ is one substituent selected from the group consisting of
(i) C₁₋₈alkyl optionally substituted with one or two substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,
wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
wherein said heteroaryl is optionally substituted on a secondary amine atom with C₁₋₈alkyl, and optionally and independently substituted on a carbon atom with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(j) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
(k) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
Y is one or two optionally present C₁₋₈alkyl substituents optionally substituted with one or two substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;
m is an integer from 2 to 5 which represents the carbon atom number corresponding to the point of attachment for the R₁—NH—C(O)-substituent moiety on the anilino ring of formula (Ia); and, n is an integer from 1 to 2.

22. The compound of claim 21, wherein when the R₁—NH—C(O)— substituent moiety is NH₂—C(O)— and R₄ is C₁₋₈alkyl, then R₄ is substituted C₁₋₈alkyl.

23. The compound of claim 21, wherein when R₄ is optionally substituted C₁₋₈alkyl, then R₅ is C₁₋₈alkyl substituted on one or two carbon atoms with one or two optionally substituted aryl substituents.

24. The compound of claim 1, wherein the compound of formula (I) is selected from a compound of formula (Ib): 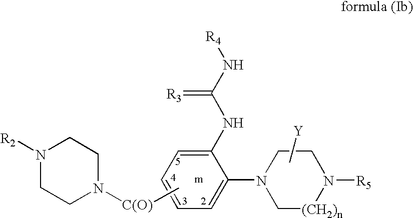
and enantiomers, diastereomers and pharmaceutically acceptable salts thereof, wherein:
(4-R₂)-1-piperazinyl-C(O)— is a substituent moiety having a variable position “m”, wherein “m” represents a carbon atom number corresponding to a point of attachment for the (4-R₂)-1-piperazinyl-C(O)— substituent moiety on the anilino ring of formula (Ib);
R₂ is selected from the group consisting of hydrogen and C₁₋₈alkyl, wherein C₁₋₈alkyl is optionally substituted with one or more substituents independently selected from the group consisting of amino, mono(C₁₋₈)alkylamino, di(C₁₋₈)alkylamino, cyano, halogen, hydroxy, nitro and carboxyl;
R₃ is selected from the group consisting of O and S;
R₄ is selected from the group consisting of
(a) C₁₋₈salkyl optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(b) carbonyl(C₁₋₈)alkyl, wherein the C₁₋₈alkyl portion of the carbonyl(C₁₋₈)alkyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(c) carbonyl(C₂₋₈)alkenyl, wherein the C₂₋₈alkenyl portion of the carbonyl(C₂₋₈)alkenyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(d) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(e) benzofused dioxolyl;
(f) benzofused dioxinyl;
(g) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(h) carbonyl-aryl, wherein the aryl portion of the carbonyl-aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
R₅ is one substituent selected from the group consisting of
(i) C₁₋₈alkyl optionally substituted with one or more substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,
wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
wherein said heteroaryl is optionally substituted on a secondary amine atom with C₁₋₈alkyl, and optionally and independently substituted on a carbon atom with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(j) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(k) aryl optionally substituted with one or more substituents independently selected from the group consisting of C₁₋₈alkyl, C₁₋₈alkoxy, amino, mono(C₁₋₄)alkylainino, di(C₁₋₄)alkylamino, cyano, halogen, hydroxy and nitro;
Y is one or more optionally present C₁₋₈alkyl substituents optionally substituted with one or more substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;
m is an integer from 2 to 5 which represents the carbon atom number corresponding to the point of attachment for the (4-R₂)-1-piperazinyl-C(O)— substituent moiety on the anilino ring of formula (Ib); and, n is an integer from 1 to 2.

25. The compound of claim 24, wherein when R₄ is optionally substituted C₁₋₈alkyl, then R₅ is C₁₋₈alkyl substituted on one or more carbon atoms with one or more optionally substituted aryl substituents.

26. The compound of claim 1, wherein the compound of formula (I) is selected from a compound of formula (Ib): 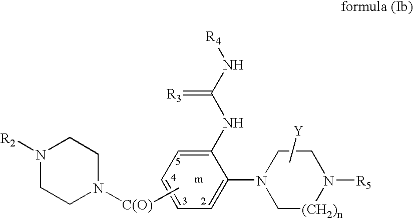
and enantiomers, diastereomers and pharmaceutically acceptable salts thereof, wherein:
(4-R₂)-1-piperazinyl-C(O)— is a substituent moiety having a variable position “m”, wherein “m” represents a carbon atom number corresponding to a point of attachment for the (4-R₂)-1-piperazinyl-C(O)— substituent moiety on the anilino ring of formula (Ib);
R₂ is selected from the group consisting of hydrogen and C₁₋₈alkyl, wherein C₁₋₈alkyl is optionally substituted with one or two substituents independently selected from the group consisting of amino, mono(C₁₋₈)alkylamino, di(C₁₋₈)alkylamino, cyano, halogen, hydroxy, nitro and carboxyl;
R₃ is selected from the group consisting of O and S;
R₄ is selected from the group consisting of
(a) C₁₋₈alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(b) carbonyl(C₁₋₈)alkyl, wherein the C₁₋₈alkyl portion of the carbonyl(C₁₋₈)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(c) carbonyl(C₂₋₈)alkenyl, wherein the C₂₋₈alkenyl portion of the carbonyl(C₂₋₈)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(d) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(e) benzofused dioxolyl;
(f) benzofused dioxinyl;
(g) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(h) carbonyl-aryl, wherein the aryl portion of the carbonyl-aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
R₅ is one substituent selected from the group consisting of
(i) C₁₋₈alkyl optionally substituted with one or two substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,
wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
wherein said heteroaryl is optionally substituted on a secondary amine atom with C₁₋₈alkyl, and optionally and independently substituted on a carbon atom with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(j) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(k) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
Y is one or two optionally present C₁₋₈alkyl substituents optionally substituted with one or two substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;
m is an integer from 2 to 5 which represents the carbon atom number corresponding to the point of attachment for the (4-R₂)-1-piperazinyl-C(O)— substituent moiety on the anilino ring of formula (Ib); and, n is an integer from 1 to 2.

27. The compound of claim 26, wherein when R₄ is optionally substituted C₁₋₈alkyl, then R₅ is C₁₋₈alkyl substituted on one carbon atom with one or two optionally substituted aryl substituents.

28. The compound of claim 1, wherein the compound of formula (I) is selected from a compound of formula (Ic): 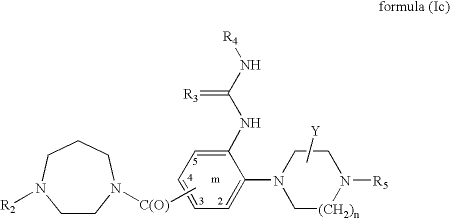
and enantiomers, diastereomers and pharmaceutically acceptable salts thereof, wherein:
(4-R₂)-hexahydro-1H-1,4-diazepin-1-yl-C(O)— is a substituent moiety having a variable position “m”, wherein “m” represents a carbon atom number corresponding to a point of attachment for the (4-R₂)-hexahydro-1H-1,4-diazepin-1-yl-C(O)— substituent moiety on the anilino ring of formula (Ic);
R₂ is selected from the group consisting of hydrogen and C₁₋₈alkyl, wherein C₁₋₈alkyl is optionally substituted with one or more substituents independently selected from the group consisting of amino, mono(C₁₋₈)alkylamino, di(C₁₋₈)alkylamino, cyano, halogen, hydroxy, nitro and carboxyl;
R₃ is selected from the group consisting of O and S;
R₄ is selected from the group consisting of
(a) C₁₋₈alkyl optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(b) carbonyl(C₁₋₈)alkyl, wherein the C₁₋₈alkyl portion of the carbonyl(C₁₋₈)alkyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(c) carbonyl(C₂₋₈)alkenyl, wherein the C₂₋₈alkenyl portion of the carbonyl(C₂₋₈)alkenyl is optionally substituted with one or more substituents independently selected from the group consisting of amino, mono(C₁₋₄)alkylamino, di(C₁₋₄)alkylamino, cyano, halogen, hydroxy, nitro and aryl, wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(d) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(e) benzofused dioxolyl;
(f) benzofused dioxinyl;
(g) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(h) carbonyl-aryl, wherein the aryl portion of the carbonyl-aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
R₅ is one substituent selected from the group consisting of
(i) C₁₋₈alkyl optionally substituted with one or more substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,
wherein said aryl is optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
wherein said heteroaryl is optionally substituted on a secondary amine atom with C₁₋₈alkyl, and optionally and independently substituted on a carbon atom with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(j) C₃₋₈cycloalkyl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(k) aryl optionally substituted with one or more substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
Y is one or more optionally present C₁₋₈alkyl substituents optionally substituted with one or more substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;
m is an integer from 2 to 5 which represents the carbon atom number corresponding to the point of attachment for the (4-R₂)-hexahydro-1H-1,4-diazepin-1-yl-C(O)— substituent moiety on the anilino ring of formula (Ic); and, n is an integer from 1 to 2.

29. The compound of claim 28, wherein when R₄ is optionally substituted C₁₋₈alkyl, then R₅ is C₁₋₈alkyl substituted on one or more carbon atoms with one or more optionally substituted aryl substituents.

30. The compound of claim 1, wherein the compound of formula (I) is selected from a compound of formula (Ic): 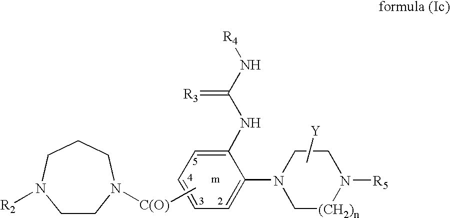
and enantiomers, diastereomers and pharmaceutically acceptable salts thereof, wherein:
(4-R₂)-hexahydro-1H-1,4-diazepin-1-yl-C(O)— is a substituent moiety having a variable position “m”, wherein “m” represents a carbon atom number corresponding to a point of attachment for the (4-R₂)-hexahydro-1H-1,4-diazepin-1-yl-C(O)— substituent moiety on the anilino ring of formula (Ic);
R₂ is selected from the group consisting of hydrogen and C₁₋₈alkyl, wherein C₁₋₈alkyl is optionally substituted with one or two substituents independently selected from the group consisting of amino, mono(C₁₋₈)alkylamino, di(C₁₋₈)alkylamino, cyano, halogen, hydroxy, nitro and carboxyl;
R₃ is selected from the group consisting of O and S;
R₄ is selected from the group consisting of
(a) C₁₋₈alkyl optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(b) carbonyl(C₁₋₈)alkyl, wherein the C₁₋₈alkyl portion of the carbonyl(C₁₋₈)alkyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(c) carbonyl(C₂₋₈)alkenyl, wherein the C₂₋₈alkenyl portion of the carbonyl(C₂₋₈)alkenyl is optionally substituted with one or two substituents independently selected from the group consisting of amino, mono(C₁₋₄)alkylamino, di(C₁₋₄)alkylamino, cyano, halogen, hydroxy, nitro and aryl, wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(d) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(e) benzofused dioxolyl;
(f) benzofused dioxinyl;
(g) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(h) carbonyl-aryl, wherein the aryl portion of the carbonyl-aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
R₅ is one substituent selected from the group consisting of
(i) C₁₋₈alkyl optionally substituted with one or two substituents independently selected from the group consisting of C₁₋₈alkoxy, amino, mono(C₁₋₄)alkylamino, di(C₁₋₄)alkylamino, cyano, halogen, hydroxy, nitro, aryl and heteroaryl,
wherein said aryl is optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and
wherein said heteroaryl is optionally substituted on a secondary amine atom with C₁₋₈alkyl, and optionally and independently substituted on a carbon atom with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
(j) C₃₋₈cycloalkyl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro; and,
(k) aryl optionally substituted with one or two substituents independently selected from the group consisting of C₁₋₈alkyl, C₁₋₈alkoxy, amino, mono(C₁₋₄)alkylamino, di(C₁₋₄)alkylamino, cyano, halogen, hydroxy and nitro;
Y is one or two optionally present C₁₋₈alkyl substituents optionally substituted with one or two substituents independently selected from the group consisting of amino, mono(C₁₋₄)alkylamino, di(C₁₋₄)alkylamino, cyano, halogen, hydroxy, nitro, C₃₋₈cycloalkyl, aryl and heteroaryl, wherein said C₃₋₈cycloalkyl, aryl and heteroaryl are optionally further substituted;
m is an integer from 2 to 5 which represents the carbon atom number corresponding to the point of attachment for the (4-R₂)-hexahydro-1H-1,4-diazepin-1-yl-C(O)— substituent moiety on the anilino ring of formula (Ic); and, n is an integer from 1 to 2.

31. The compound of claim 30, wherein when R₄ is optionally substituted C₁₋₈alkyl, then R₅ is C₁₋₈alkyl substituted on one carbon atom with one or two optionally substituted aryl substituents.

32. A compound selected from the group consisting of:
4-[4-(2-methoxyphenyl)-1-piperazinyl]-3-[[(phenylamino)carbonyl]amino]-benzamide,
3-[[(phenylamino)carbonyl]amino]-4-[4-(phenylmethyl)-1-piperazinyl]-benzamide,
4-[4-[bis(4-fluorophenyl)methyl]-1-piperazinyl]-3-[[(phenylamino)carbonyl]amino]-benzamide,
4-[4-[bis(4-fluorophenyl)methyl]-1-piperazinyl]-3-[[[(2-fluorophenyl)amino]carbonyl]amino]-benzamide,
4-[4-(diphenylmethyl)-1-piperazinyl]-3-[[[(4-nitrophenyl)amino]carbonyl]amino]-benzamide,
4-[4-(diphenylmethyl)-1-piperazinyl]-3-[[[(phenylmethyl)amino]carbonyl]amino]-benzamide,
3-[[[(3,5-dimethylphenyl)amino]carbonyl]amino]-4-[4-(diphenylmethyl)-1-piperazinyl]-benzamide,
4-[4-(diphenylmethyl)-1-piperazinyl]-3-[[(phenylamino)carbonyl]amino]-benzamide,
4-[4-(9H-fluoren-9-yl)-1-piperazinyl]-3-[[(phenylamino)carbonyl]amino]-benzamide,
3-[[(cyclohexylamino)carbonyl]amino]-4-[4-(diphenylmethyl)-1-piperazinyl]-benzamide,
4-[4-(diphenylmethyl)-1-piperazinyl]-3-[[[[(1S)-1-phenylethyl]amino]carbonyl]amino]-benzamide,
3-[[(butylamino)carbonyl]amino]-4-[4-(diphenylmethyl)-1-piperazinyl]-benzamide,
4-[4-(diphenylmethyl)-1-piperazinyl]-3-[[[(4-fluorophenyl)amino]carbonyl]amino]-benzamide,
3-[[(1,3-benzodioxol-5-ylamino)carbonyl]amino]-4-[4-[bis(4-fluorophenyl)methyl]-1-piperazinyl]-benzamide,
4-[4-[bis(4-fluorophenyl)methyl]-1-piperazinyl]-3-[[[(2,4-dimethylphenyl)amino]carbonyl]amino]-benzamide,
4-[4-[bis(4-fluorophenyl)methyl]-1-piperazinyl]-3-[[[(1-phenylethyl)amino]carbonyl]amino]-benzamide,
4-[4-[bis(4-fluorophenyl)methyl]-1-piperazinyl]-3-[[[(2-methoxyphenyl)amino]carbonyl]amino]-benzamide,
4-[4-[bis(4-fluorophenyl)methyl]-1-piperazinyl]-3-[[[(2,4-dimethoxyphenyl)amino]carbonyl]amino]-benzamide,
4-[4-[bis(4-fluorophenyl)methyl]-1-piperazinyl]-3-[[[[4-(dimethylamino)phenyl]amino]carbonyl]amino]-benzamide,
4-[4-[bis(4-fluorophenyl)methyl]-1-piperazinyl]-3-[[[(4-methoxyphenyl)amino]carbonyl]amino]-benzamide,
4-[4-[bis(4-fluorophenyl)methyl]-1-piperazinyl]-3-[[[(phenylmethyl)amino]thioxomethyl]amino]-benzamide,
4-[4-[bis(4-fluorophenyl)methyl]-1-piperazinyl]-3-[[(phenylamino)thioxomethyl]amino]-benzamide,
N-[2-[4-(diphenylmethyl)-1-piperazinyl]-5-[(4-methyl-1-piperazinyl)carbonyl]phenyl]-N′-phenylurea,
N-[2-[4-(diphenylmethyl)-1-piperazinyl]-5-[(hexahydro-1H-1,4-diazepin-1-yl)carbonyl]phenyl]-N′-phenylurea,
N-cyclohexyl-N′-[2-[4-(diphenylmethyl)-1-piperazinyl]-5-[(hexahydro-1H-1,4-diazepin-1-yl)carbonyl]-phenyl]urea,
N-(2-aminoethyl)-4-[4-(diphenylmethyl)-1-piperazinyl]-3-[[(phenylamino)carbonyl]amino]-benzamide,
N-(2-aminoethyl)-3-[[(cyclohexylamino)carbonyl]amino]-4-[4-(diphenylmethyl)-1-piperazinyl]-benzamide,
N-cyclohexyl-N′-[2-[4-(diphenylmethyl)-1-piperazinyl]-5-[(4-methyl-1-piperazinyl)carbonyl]-phenyl]urea,
N-[2-(dimethylamino)ethyl]-4-[4-(diphenylmethyl)-1-piperazinyl]-3-[[(phenylamino)carbonyl]amino]-benzamide,
3-[[(cyclohexylamino)carbonyl]amino]-N-[2-(dimethylamino)ethyl]-4-[4-(diphenylmethyl)-1-piperazinyl]-benzamide,
N-cyclohexyl-N′-[2-[4-(diphenylmethyl)-1-piperazinyl]-5-[(hexahydro-4-methyl-1H-1,4-diazepin-1-yl)carbonyl]phenyl]-urea,
N-[4-[4-(diphenylmethyl)-1-piperazinyl]-3-[[(phenylamino)carbonyl]amino]benzoyl]-L-leucine,
N-cyclohexyl-N′-[2-[4-(diphenylmethyl)-1-piperazinyl]-5-(1-piperazinylcarbonyl)phenyl]-urea,
N-[2-[4-(diphenylmethyl)-1-piperazinyl]-5-(1-piperazinylcarbonyl)phenyl]-N′-(phenylmethyl)urea,
N-[2-[4-(diphenylmethyl)-1-piperazinyl]-5-(1-piperazinylcarbonyl)phenyl]-N′-phenylurea,
N-(2,4-dimethylphenyl)-N′-[2-[4-(diphenylmethyl)-1-piperazinyl]-5-(1-piperazinylcarbonyl)phenyl]-urea,
N-(3,5-dimethylphenyl)-N′-[2-[4-(diphenylmethyl)-1-piperazinyl]-5-(1-piperazinylcarbonyl)phenyl]-urea,
N-[2-[4-(diphenylmethyl)-1-piperazinyl]-5-(1-piperazinylcarbonyl)phenyl]-N′-(4-methoxyphenyl)urea,
N-[2-[4-(9H-fluoren-9-yl)-1-piperazinyl]-5-(1-piperazinylcarbonyl)phenyl]-N′-phenylurea,
N-[2-[4-[(4-chlorophenyl)phenylmethyl]-1-piperazinyl]-5-(1-piperazinylcarbonyl)phenyl]-N′-cyclohexyl-urea,
N-[2-[4-[bis(4-fluorophenyl)methyl]-1-piperazinyl]-5-(1-piperazinylcarbonyl)phenyl]-N′-phenylurea,
N-[2-[4-[bis(4-fluorophenyl)methyl]-1-piperazinyl]-5-(1-piperazinylcarbonyl)phenyl]-N′-cyclohexylurea,
N-phenyl-N′-[2-[4-(1-phenylethyl)-1-piperazinyl]-5-(1-piperazinylcarbonyl)phenyl]-urea,
N-[2-[4-(2-methoxyphenyl)-1-piperazinyl]-5-(1-piperazinylcarbonyl)phenyl]-N′-phenylurea,
N-phenyl-N′-[2-[4-(phenylmethyl)-1-piperazinyl]-5-(1-piperazinylcarbonyl)phenyl]-urea,
N-[2-(4-cyclohexyl-1-piperazinyl)-5-(1-piperazinylcarbonyl)phenyl]-N′-phenylurea,
N-cyclohexyl-N′-[2-[4-(phenylmethyl)-1-piperazinyl]-5-(1-piperazinylcarbonyl)phenyl]-urea,
N-cyclohexyl-N′-[2-[4-(1-phenylethyl)-1-piperazinyl]-5-(1-piperazinylcarbonyl)phenyl]-urea,
N-cyclohexyl-N′-[2-(4-cyclohexyl-1-piperazinyl)-5-(1-piperazinylcarbonyl)phenyl]-urea,
N-cyclohexyl-N′-[2-[4-(2-methoxyphenyl)-1-piperazinyl]-5-(1-piperazinylcarbonyl)phenyl]-urea,
N-butyl-N′-[2-[4-(diphenylmethyl)-1-piperazinyl]-5-(1-piperazinylcarbonyl)phenyl]-urea,
N-[2-[4-(diphenylmethyl)-1-piperazinyl]-5-(1-piperazinylcarbonyl)phenyl]-N′-(2-fluorophenyl)urea,
N-[4-(dimethylamino)phenyl]-N′-[2-[4-(diphenylmethyl)-1-piperazinyl]-5-(1-piperazinylcarbonyl)phenyl]-urea,
N-[2-[4-(diphenylmethyl)-1-piperazinyl]-5-(1-piperazinylcarbonyl)phenyl]-N′-(2-methoxy phenyl)-urea,
N-[2-[4-[(4-chlorophenyl)phenylmethyl]-1-piperazinyl]-5-(1-piperazinylcarbonyl)phenyl]-N′-phenyl-urea,
N-[2-[4-(diphenylmethyl)-1-piperazinyl]-5-(1-piperazinylcarbonyl)phenyl]-N′-[(2E)-1-oxo-3-phenyl-2-propenyl]-urea,
N-(1,1-dimethylethyl)-N′-[2-[4-(diphenylmethyl)-1-piperazinyl]-5-(1-piperazinylcarbonyl) phenyl]-urea,
N-cyclopentyl-N′-[2-[4-(diphenylmethyl)-1-piperazinyl]-5-(1-piperazinylcarbonyl)phenyl]-urea,
N-[2-[4-(diphenylmethyl)-1-piperazinyl]-5-(1-piperazinylcarbonyl)phenyl]-N′-[(1S)-1-phenylethyl]-urea,
N-[2-[4-(diphenylmethyl)-1-piperazinyl]-5-(1-piperazinylcarbonyl)phenyl]-N′-(phenylmethyl) thiourea,
N-[2-[4-(diphenylmethyl)-1-piperazinyl]-5-(1-piperazinylcarbonyl)phenyl]-N′-(1-methylethyl)urea,
N-(4-chlorobenzoyl)-N′-[2-[4-(diphenylmethyl)-1-piperazinyl]-5-(1-piperazinylcarbonyl) phenyl]-thiourea,
4-[4-[(4-fluorophenyl)-4-pyridinylmethyl]-1-piperazinyl]-3-[[(phenylamino)carbonyl]amino]-benzamide,
3-[[(cyclohexylamino)carbonyl]amino]-4-[4-[(4-fluorophenyl)-4-pyridinylmethyl]-1-piperazinyl]-benzamide,
4-[4-[(4-fluorophenyl)-4-pyridinylmethyl]hexahydro-1H-1,4-diazepin-1-yl]-3-[[(phenylamino)carbonyl]amino]-benzamide,
3-[[(cyclohexylamino)carbonyl]amino]-4-[4-[(4-fluorophenyl)-4-pyridinylmethyl]hexahydro-1H-1,4-diazepin-1-yl]-benzamide,
4-[4-[bis(4-fluorophenyl)methyl]hexahydro-1H-1,4-diazepin-1-yl]-3-[[(phenylamino)carbonyl]amino]-benzamide,
4-[4-[bis(4-fluorophenyl)methyl]hexahydro-1H-1,4-diazepin-1-yl]-3-[[(cyclohexylamino)carbonyl]amino]-benzamide,
N-[2-[4-[bis(4-fluorophenyl)methyl]hexahydro-1H-1,4-diazepin-1-yl]-4-(1-piperazinylcarbonyl)phenyl]-N′-phenyl-urea,
N-[2-[4-[bis(4-fluorophenyl)methyl]-1-piperazinyl]-4-(1-piperazinylcarbonyl)phenyl]-N′-phenyl-urea,
N-[2-[4-(diphenylmethyl)-1-piperazinyl]-4-(1-piperazinylcarbonyl)phenyl]-N′-phenyl-urea,
N-cyclohexyl-N′-[2-[4-(diphenylmethyl)-1-piperazinyl]-4-(1-piperazinylcarbonyl)phenyl]-urea, and
3-[4-(diphenylmethyl)-1-piperazinyl]-4-[[(phenylamino)carbonyl]amino]-benzamide.

33. A composition comprising a pharmaceutically acceptable carrier, excipient, tableting ingredient or diluent and the compound of claim 1.

34. A method of treating or preventing a disease or condition in a subject which disease or condition is affected by phospholipase modulation, which method comprises administering to the subject in need of such treatment or prevention a therapeutically effective amount of the compound of claim 1.

35. The method of claim 34, wherein the method further comprises administering to the subject in need of such treatment or prevention a therapeutically effective amount of the composition of claim 33.

36. A method of treating or ameliorating an inflammatory disorder in a subject in need thereof comprising administering to the subject a therapeutically effective amount of the compound of claim 1.

37. The method of claim 36, wherein the method further comprises administering to the subject a therapeutically effective amount of the composition of claim 33.

38. A method of treating or ameliorating restenosis in a subject in need thereof comprising administering to the subject a therapeutically effective amount of the compound of claim 1 by impregnating the therapeutically effective amount of said compound on the surface of a medical device and administering the medical device to the subject.

39. The method of claim 38, wherein the method further comprises a therapeutically effective amount of the composition of claim 33 impregnated on the surface of said medical device.
